(19) United States
(12) Patent Application Publication
(10) Pub. No.: US 2015/0159134 A1
(43) Pub. Date: Jun. 11, 2015

(54) METHOD FOR PRODUCING RETINAL PIGMENT EPITHELIAL CELLS

(71) Applicant: PFIZER LIMITED, SANDWICH (GB)

(72) Inventors: PARUL CHOUDHARY, GREAT ABINGTON (GB); BEATA SURMACZ-CORDLE, GREAT ABINGTON (GB); HEATHER DAWN ELLEN FOX, GREAT ABINGTON (GB); PAUL JOHN WHITING, GREAT ABINGTON (GB)

(73) Assignee: PFIZER LIMITED, SANDWICH (GB)

(21) Appl. No.: 14/566,184

(22) Filed: Dec. 10, 2014

Related U.S. Application Data
(60) Provisional application No. 61/914,445, filed on Dec. 11, 2013.

(30) Foreign Application Priority Data
Dec. 8, 2014 (IB) PCT/IB2014/066703

(51) Int. Cl. C12N 5/079; A61K 35/30

(52) U.S. Cl. CPC C12N 5/0621; A61K 35/30; C12N 2506/02; C12N 2500/42; C12N 2500/46

(57) ABSTRACT

The invention relates to a method for producing retinal pigment epithelial cells.

This application claims the benefit of U.S. Provisional Application No. 61/914,445 filed on Dec. 11, 2013, and International Application No. PCT/IB2014/066703, filed Dec. 8, 2014, each of which is hereby incorporated by reference in its entirety.

### FIELD OF THE INVENTION

The invention relates to methods for producing retinal pigment epithelial (RPE) cells from pluripotent cells. The invention also relates to the cells obtained or obtainable by such methods as well as to their use for the treatment of retinal diseases. The invention also relates to a process for expanding RPE cells.

### BACKGROUND OF THE INVENTION

The retinal pigment epithelium is the pigmented cell layer outside the neurosensory retina between the underlying choroid (the layer of blood vessels behind the retina) and overlying retinal visual cells (e.g., photoreceptors rods and cones). The retinal pigment epithelium is critical to the function and health of photoreceptors and the retina. The retinal pigment epithelium maintains photoreceptor function by recycling photopigments, delivering, metabolizing, and storing vitamin A, phagocytosing rod photoreceptor outer segments, transporting iron and small molecules between the retina and choroid, maintaining Bruch's membrane and absorbing stray light to allow better image resolution. Degeneration of the retinal pigment epithelium can cause retinal detachment, retinal dysplasia, or retinal atrophy that is associated with a number of vision-altering ailments that result in photoreceptor damage and blindness, such as, choroideremia, diabetic retinopathy, macular degeneration (including age-related macular degeneration), retinitis pigmentosa, and Stargardt's Disease.

A potential treatment for such diseases is the transplantation of RPE cells into the retina of those affected with the diseases. It is believed that replenishment of retinal pigment epithelial cells by their transplantation may delay, halt or reverse degeneration, improve retinal function and prevent blindness stemming from such conditions. It has been demonstrated in animal models that photoreceptor rescue and preservation of visual function could be achieved by subretinal transplantation of RPE cells (see for example Coffey, P J et al. Nat. Neurosci. 2002:5, 53-56; Sauve, Yet al. Neuroscience 2002: 114, 389-401). Therefore, there is a high interest in finding ways to produce RPE cells, for example from pluripotent cells, as a source for cell transplantation for the treatment of retinal diseases.

The potential of mouse and non-human primate embryonic stem cells to differentiate into RPE cells, and to survive and attenuate retinal degeneration after transplantation, has been demonstrated. Spontaneous differentiation of human embryonic stem cells into RPE cells was shown (see for example WO2005/070011). However, the efficiency and reproducibility of such process was low. Therefore, there is a need for methods for producing RPE cells which are well controlled, reproducible, efficient and/or suitable for scale up and for producing RPE cells for drug screening, disease modeling and/or therapeutic use.

### SUMMARY OF THE INVENTION

The present invention relates to methods for producing RPE cells. It is demonstrated that the methods provide robust and reproducible differentiation of pluripotent cells such as human embryonic stem cells (hESCs) to give rise to RPE cells. In addition, the methods provided herein are easily scalable to give a high yield of RPE cells. Methods disclosed herein can be used, for example without limitation, for reproducibly and efficiently differentiating pluripotent cells such as hESC into RPE cells in xeno-free conditions.

Methods for producing RPE cells are provided herein. In some embodiments, the method comprises the steps of:

(a) culturing pluripotent cells in the presence of a first SMAD inhibitor and a second SMAD inhibitor;

(b) culturing the cells of step (a) in the presence of a Bone Morphogenetic Protein (BMP) pathway activator and in the absence of the first and second SMAD inhibitors; and,

(c) replating the cells of step (b).

In some embodiments of said method, the method further comprises the following steps:

(d) culturing the replated cells of step (c) in the presence of an activin pathway activator;

(e) replating the cells of step (d); and,

(f) culturing the replated cells of step (e).

In another embodiment of said method,

step (b) further comprises, after culturing the cells in the presence of the BMP pathway activator, culturing the cells for at least 10 days in the absence of the BMP pathway activator;

step (c) comprises replating the cells of step (b) having a cobblestone morphology; and said method further comprising the step of:

(d) culturing the replated cells of step (c).

Also provided are methods for expanding RPE cells. In some embodiments, the method comprises the following steps:

(a) plating RPE cells at a density between 1000 and 100000 cells/cm², and,

(b) culturing said RPE cells in the presence of SMAD inhibitor, cAMP or an agent which increases the intracellular concentration of cAMP.

Also provided are methods for purifying RPE cells comprising:

a) providing a cell population comprising RPE cells and non RPE cells;

b) increasing the percentage of RPE cells in the cell population by enriching the cell population for cells expressing CD59.

Also provided are RPE cells obtained or obtainable by a method disclosed herein.

Also provided are pharmaceutical compositions. The pharmaceutical compositions comprise RPE cells suitable for transplantation into the eye of a subject affected with a retinal disease. In some embodiments, the pharmaceutical composition comprises a structure suitable for supporting RPE cells. In some embodiments, the pharmaceutical composition comprises a porous membrane and RPE cells. In some embodiments, the pores of the membrane are between about 0.2 μm and about 0.5 μm in diameter and the pore density are between about 1×10⁷ and about 3×10⁸ pores per cm². In some embodiments, the membrane is coated on one side with a coating supporting RPE cells. In some embodiments, the coating comprises a glycoprotein, preferably selected from laminin or vitronectin. In some embodiments, the coating comprises vitronectin. In some embodiments, the membrane is made of polyester.

Also provided are methods for the treatment of a retinal disease in a subject. In some embodiments, the method comprises administering RPE cells of the present invention to a subject affected by or at risk for retinal disease, thereby treating the retinal disease.

Also provided are methods for producing retinal pigment epithelial (RPE) cells comprising the steps of:

(a) culturing pluripotent cells in the presence of a first SMAD inhibitor and a second SMAD inhibitor;

(b) culturing the cells of step (a) in the presence of a BMP pathway activator and in the absence of the first and second SMAD inhibitors; and,

(c) replating the cells of step (b).

In some embodiments, in step (a), the cells are cultured as a monolayer. In some embodiments, in step (b), the cells are cultured as a monolayer. In some embodiments, in step (a), the cells are cultured in a suspension culture. In some embodiments, in step (b), the cells are cultured in a suspension culture. In some embodiments, the pluripotent cells are selected from embryonic stem cells or induced pluripotent stem cells. In some embodiments, the pluripotent cells are human cells. In some embodiments, the pluripotent cells are human embryonic stem cells. In some embodiments, the pluripotent cells are human induced pluripotent stem cells. In some embodiments, the pluripotent cells are obtained by means which do not require the destruction of a human embryo. In some embodiments, the first SMAD inhibitor is an inhibitor of BMP type 1 receptor ALK2. In some embodiments, the first SMAD inhibitor is an inhibitor of BMP type 1 receptors ALK2 and ALK3. In some embodiments, the first SMAD inhibitor prevents Smad1, Smad5 and/or Smad8 phosphorylation. In some embodiments, the first SMAD inhibitor is a dorsomorphin derivative. In some embodiments, the first SMAD inhibitor is selected from dorsomorphin, noggin or chordin. In some embodiments, the first SMAD inhibitor is 4-(6-(4-(piperazin-1-yl)phenyl)pyrazolo[1,5-a]pyrimidin-3-yl)quinoline (LDN193189) or a salt or hydrate thereof. In some embodiments, the concentration of first SMAD inhibitor is between 0.5 nM and 10 μM. In some embodiments, in step (a), the concentration of first SMAD inhibitor is between 500 nM and 2 μM. In some embodiments, in step (a), the concentration of first SMAD inhibitor is about 1 μM. The method according to any one of claims 1 to 19 wherein the second SMAD inhibitor is an inhibitor of ALK5. In some embodiments, the second SMAD inhibitor is an inhibitor of ALK5 and ALK4. In some embodiments, the second SMAD inhibitor is an inhibitor of ALK5 and ALK4 and ALK7. In some embodiments, the second SMAD inhibitor is selected from:
4-(4-(benzo[d][1,3]dioxol-5-yl)-5-(pyridin-2-yl)-1H-imidazol-2-yl)benzamide;
2-methyl-5-(6-(m-tolyl)-1H-imidazo[1,2-a]imidazol-5-yl)-2H-benzo[d][1,2,3]triazole;
2-(6-methylpyridin-2-yl)-N-(pyridin-4-yl)quinazolin-4-amine; 2-(3-(6-methylpyridin-2-yl)-1H-pyrazol-4-yl)-1,5-naphthyridine;
4-(2-(6-methylpyridin-2-yl)-5,6-dihydro-4H-pyrrolo[1,2-b]pyrazol-3-yl)phenol;
2-(4-methyl-1-(6-methylpyridin-2-yl)-1H-pyrazol-5-yl)thieno[3,2-c]pyridine;
4-(5-(3,4-dihydroxyphenyl)-1-(2-hydroxyphenyl)-1H-pyrazol-3-yl)benzamide;
2-(5-chloro-2-fluorophenyl)-N-(pyridin-4-yl)pteridin-4-amine;
6-methyl-2-phenylthieno[2,3-d]pyrimidin-4(3H)-one;
3-(6-Methyl-2-pyridinyl)-N-phenyl-4-(4-quinolinyl)-1H-pyrazole-1-carbothioamide (A 83-01); 2-(5-Benzo[1,3]dioxol-5-yl-2-tert-butyl-3H-imidazol-4-yl)-6-methylpyridine (SB-505124);
7-(2-morpholinoethoxy)-4-(2-(pyridin-2-yl)-5,6-dihydro-4H-pyrrolo[1,2-b]pyrazol-3-yl)quinoline (LY2109761);
4-[3-(2-pyridinyl)-1H-pyrazol-4-yl]-quinoline (LY364947); and,
4-(4-(benzo[d][1,3]dioxol-5-yl)-5-(pyridin-2-yl)-1H-imidazol-2-yl)benzamide (SB-431542) or a salt or hydrate thereof.

In some embodiments, the second SMAD inhibitor is 4-(4-(benzo[d][1,3]dioxol-5-yl)-5-(pyridin-2-yl)-1H-imidazol-2-yl)benzamide (SB-431542). In some embodiments, in step (a), the concentration of second SMAD inhibitor is between 0.5 nM and 100 μM. In some embodiments, in step (a), the concentration of second SMAD inhibitor is between 1 μM and 50 μM. In some embodiments, in step (a), the concentration of second SMAD inhibitor is about 10 μM. In some embodiments, in step (a), the pluripotent cells are cultured for at least 1 day. In some embodiments, in step (a), the pluripotent cells are cultured for at least 2 days. In some embodiments, in step (a), the pluripotent cells are cultured for between 2 and 10 days. In some embodiments, in step (a), the pluripotent cells are cultured for between 3 and 5 days. In some embodiments, in step (a), the pluripotent cells are cultured for about 4 days. In some embodiments, before step (a), the cells are cultured as a monolayer at an initial density of at least 1000 cells/cm². In some embodiments, before step (a), the cells are cultured as a monolayer at an initial density of between 100000 and 500000 cells/cm².

In some embodiments, the BMP pathway activator comprises a BMP. In some embodiments, the BMP pathway activator comprises a BMP selected from BMP2, BMP3, BMP4, BMP6, BMP7, BMP8, BMP9, BMP10, BMP11 or BMP15. In some embodiments, the BMP pathway activator is a BMP homodimer. In some embodiments, the BMP pathway activator is a BMP heterodimer. In some embodiments, the BMP pathway activator is a BMP2/6 heterodimer, a BMP4/7 heterodimer or a BMP3/8 heterodimer. In some embodiments, the BMP pathway activator is a BMP4/7 heterodimer.

In some embodiments, in step (b), the concentration of BMP pathway activator is between 1 ng/mL and 10 μg/mL. In some embodiments, in step (b), the concentration of BMP pathway activator is between 50 ng/mL and 500 ng/mL. In some embodiments, in step (b), the concentration of BMP pathway activator is about 100 ng/mL. In some embodiments, in step (b), said cells are cultured for at least 1 day. In some embodiments, in step (b), said cells are cultured for between 2 days and 20 days. In some embodiments, in step (b), said cells are cultured for about 3 days.

In some embodiments, in step (c), said cells are replated at a density of at least 1000 cells/cm². In some embodiments, in step (c), said cells are replated at a density of between 100000 and 1000000 cells/cm². In some embodiments, in step (c), said cells are replated at a density of about 500000 cells/cm². In some embodiments, in step (c), said cells are replated on Matrigel®, fibronectin or Cellstart®.

Also provided herein are methods for producing RPE cells comprising the steps of:

(a) culturing pluripotent cells in the presence of a first SMAD inhibitor and a second SMAD inhibitor;

(b) culturing the cells of step (a) in the presence of a BMP pathway activator and in the absence of the first and second SMAD inhibitors;

(c) replating the cells of step (b);

(d) culturing the replated cells of step (c) in the presence of an activin pathway activator;

(e) replating the cells of step (d); and,

(f) culturing the replated cells of step (e).

In some embodiments, the cells are cultured for at least 1 day. In some embodiments, in step (d), the cells are cultured for at least 3 days. In some embodiments, in step (d), the cells are cultured for between 3 and 20 days. In some embodiments, in step (d), the concentration of activin pathway activator is between 1 ng/mL and 10 μg/mL. In some embodiments, in step (d), the concentration of activin pathway activator is about 100 ng/mL. In some embodiments, in step (d), the activin pathway activator is activin A. In some embodiments, in step (d), the cells are cultured in the presence of cAMP. In some embodiments, in step (d), the concentration of cAMP is about 0.5 mM.

In some embodiments, in step (e), the cells are replated at a density of at least 1000 cells/cm². In some embodiments, in step (e), said cells are replated at a density of between 20000 and 500000 cells/cm². In some embodiments, in step (e), said cells are replated at a density of about 200000 cells/cm². In some embodiments, in step (e), said cells are replated on Matrigel®, fibronectin or Cellstart®.

In some embodiments, in step (f), the cells are cultured for at least 5 days. In some embodiments, in step (f), the cells are cultured for at least 14 days. In some embodiments, in step (f), the cells are cultured for between 10 and 35 days. In some embodiments, in step (f), the cells are cultured for about 28 days. In some embodiments, in step (f), the cells are cultured in the presence of cAMP. In some embodiments, in step (f), the concentration of cAMP is about 0.5 mM.

In some embodiments, step (b) further comprises, after culturing the cells in the presence of the BMP pathway activator, culturing the cells for at least 10 days in the absence of the BMP pathway activator; step (c) comprises replating the cells of step (b) having a cobblestone morphology; and said method further comprising the step of: (d) culturing the replated cells of step (c). In some embodiments, in step (b), the cells are cultured for at least 20 days in the absence of BMP pathway activator. In some embodiments, in step (b), the cells are cultured for between 30 and 50 days in the absence of BMP pathway activator. In some embodiments, in step (b), the cells are cultured for about 40 days in the absence of BMP pathway activator. In some embodiments, in step (c), the cells are replated at a density of at least 1000 cells/cm². In some embodiments, in step (c), the cells are replated at a density of between 50000 and 500000 cells/cm². In some embodiments, in step (c), the cells are replated at a density of about 200000 cells/cm². In some embodiments, in step (c), said cells are replated on Matrigel®, fibronectin or Cellstart®. In some embodiments, in step (d), the cells are cultured for at least 5 days. In some embodiments, in step (d), the cells are cultured for between 10 and 40 days. In some embodiments, in step (d), the cells are cultured for about 14 days. In some embodiments, in step (d), the cells are cultured in the presence of cAMP. In some embodiments, in step (d), the concentration of cAMP is about 0.5 mM.

Also provided are methods for producing RPE cells comprising the steps of:

(a) culturing pluripotent cells in the presence of a first SMAD inhibitor and a second SMAD inhibitor;

(b) culturing the cells of step (a) in the presence of a BMP pathway activator and in the absence of the first and second SMAD inhibitors;

(c) replating the cells of step (b);

(d) culturing the replated cells of step (c) in the presence of an activin pathway activator;

(e) replating the cells of step (d);

(f) culturing the replated cells of step (e).

In some embodiments, in step (e), the cells are replated at a density of at least 1000 cells/cm². In some embodiments, in step (e), the cells are replated at a density of between 50000 and 500000 cells/cm². In some embodiments, in step (e), the cells are replated at a density of about 200000 cells/cm². In some embodiments, in step (e), said cells are replated on Matrigel®, fibronectin or Cellstart®. In some embodiments, in step (f), the cells are cultured for at least 10 days. In some embodiments, in step (f), the cells are cultured for between 15 and 40 days. In some embodiments, in step (f), the cells are cultured for about 28 days.

In some embodiments, a method for producing RPE cells provided herein further comprises the step of harvesting the RPE cells.

In some embodiments, a method for producing RPE cells provided herein further comprises the step of purifying the RPE cells. In some embodiments, a step of purifying the RPE cells comprises:
contacting the cells with an anti-CD59 antibody conjugated to a fluorophore, and,
selecting the cells that bind to the anti-CD59 antibody using FACS.

In some embodiments, a step of purifying the RPE cells comprises:
contacting the cells with an anti-CD59 antibody conjugated to a magnetic particle, and,
selecting the cells that bind to the anti-CD59 antibody using MACS.

In some embodiments, a method for producing RPE cells provided herein further comprises the step of purifying the RPE cells by Fluorescence Activated Cell Sorting (FACS) or Magnetic Activated Cell Sorting (MACS).

In some embodiments, in all steps of a method for producing RPE cells provided herein, the cells are cultured as a monolayer.

In some embodiments, the RPE cells are expanded by a method comprising
replating RPE cells; and,
culturing the replated RPE cells.

In some embodiments, the cells are replated at a density between 1000 and 100000 cells/cm². In some embodiments, the cells are replated at a density between 10000 and 30000 cells/cm². In some embodiments, the cells are replated at a density of about 20000 cells/cm². In some embodiments, the cells are replated on Matrigel®, Fibronectin or Cellstart®.

In some embodiments, the cells are cultured for at least 7 days, at least 14 days, at least 28 days or at least 42 days. In some embodiments, the cells are cultured for about 49 days. In some embodiments, the cells are cultured in the presence of a SMAD inhibitor, cAMP or an agent which increases the intracellular concentration of cAMP. In some embodiments, the agent is selected from an Adenyl Cyclase activator, preferably forskolin or a phosphodiesterase (PDE) inhibitor, preferably a PDE1, PDE2, PDE3, PDE4, PDE7, PDE8, PDE10 and/or PDE11 inhibitor.

In some embodiments, the cells are cultured in the presence of cAMP. In some embodiments, the concentration of cAMP is between 0.01 mM and 1M. In some embodiments, the concentration of cAMP is about 0.5 mM.

Also provided are methods for producing RPE cells comprising the steps of:

(a) plating RPE cells at a density of at least 1000 cells/cm², and,

(b) culturing said RPE cells in the presence of SMAD inhibitor, cAMP or an agent which increases the intracellular concentration of cAMP. In some embodiments, in step (a), the cells are plated at a density between 5000 and 100000 cells/cm². In some embodiments, in step (a), the cells are plated at a density about 20000 cells/cm². In some embodiments, in step (a), the cells are plated on Matrigel®, Fibronectin or Cellstart®. In some embodiments, in step (b), the cells are cultured for at least 7 days, at least 14 days, at least 28 days or at least 42 days. In some embodiments, in step (b), the cells are cultured for about 49 days. In some embodiments, the agent is selected from an adenyl Cyclase activator, preferably forskolin or a PDE inhibitor, preferably a PDE1, PDE2, PDE3, PDE4, PDE7, PDE8, PDE10 and/or PDE11 inhibitor. In some embodiments, in step (b), the cells are cultured in the presence of cAMP. In some embodiments, the concentration of cAMP is between 0.01 mM and 1M. In some embodiments, the concentration of cAMP is about 0.5 mM. In some embodiments, in step (b), the cells are cultured in the presence of a SMAD inhibitor. In some embodiments, the SMAD inhibitor is 2-(6-methylpyridin-2-yl)-N-(pyridin-4-yl)quinazolin-4-amine, 6-(1-(6-methylpyridin-2-yl)-1H-pyrazol-5-yl)quinazolin-4(3H)-one, or 4-methoxy-6-(3-(6-methylpyridin-2-yl)-1H-pyrazol-4-yl)quinoline. In some embodiments, the produced RPE cells have a cobblestone morphology, are pigmented and express at least one of the following RPE markers: MITF, PMEL17, CRALBP, MERTK, BEST1 and ZO-1. In some embodiments, the produced RPE cells secrete VEGF and PEDF.

In some embodiments of a method for producing RPE cells provided herein, all steps are carried out in xeno-free conditions. Also provided herein are RPE cells obtained by a method provided herein. Also provided herein RPE cells obtainable by a method provided herein. Also provided herein are pharmaceutical compositions such RPE cells.

Also provided are methods for the treatment of a retinal disease in a subject, said method comprising administering RPE cells provided herein, or a pharmaceutical composition provided herein.

Also provided are methods for producing RPE cells comprising:

a) providing a population of pluripotent cells;

b) inducing the differentiation of pluripotent cells into RPE cells, and,

c) enriching the cell population for cells expressing CD59.

In some embodiments, step c) comprises
contacting the cells with an anti-CD59 antibody conjugated to a fluorophore, and,
selecting the cells that bind to the anti-CD59 antibody using FACS.

In some embodiments, step c) comprises
contacting the cells with an anti-CD59 antibody conjugated to a magnetic particle, and,
selecting the cells that bind to the anti-CD59 antibody using MACS.

Also provided are methods for purifying RPE cells comprising:

a) providing a cell population comprising RPE cells and non RPE cells;

b) increasing the percentage of RPE cells in the cell population by enriching the cell population for cells expressing CD59.

In some embodiments, step b) comprises
contacting the cell population with an anti-CD59 antibody conjugated to a fluorophore, and,
selecting the cells that bind to the anti-CD59 antibody using FACS.

In some embodiments, step b) comprises
contacting the cell population with an anti-CD59 antibody conjugated to a magnetic particle, and,
selecting the cells that bind to the anti-CD59 antibody using MACS.

In some embodiments, the non RPE cells are pluripotent cells or RPE progenitors.

### BRIEF DESCRIPTION OF DRAWINGS

FIG. 1A shows a schematic representation of a specific example of the early and late replating methods.

FIGS. 1B and 1C show graphs indicating the percentage of cells expressing PAX6 and OCT4 as measured by immunocytochemistry at different time points during treatment with SMAD inhibitors. FIG. 1B: samples induced with LDN/SB. FIG. 1C: samples not induced with LDN/SB.

FIG. 1D shows graphs indicating the percentage of cells expressing PAX6 (top graph) and OCT4 (Bottom graph) as measured by immunocytochemistry after 2 days (LDN/SB 2D) or 5 days (Control+) treatment with SMAD inhibitors.

FIG. 2A shows graphs indicating the relative expression of Mitf (top graph) and Silv (PMEL17) (bottom graph) as measured by qPCR under different conditions. FIG. 2B shows graphs indicating the percentage of cells expressing MITF (top graph) and PMEL17 (bottom graph) as measured by immunocytochemistry. FIGS. 2A and 2B show that treatment with a BMP pathway activator after step (a) is essential to induce the expression of MITF and PMEL17.

FIG. 3 shows graphs indicating the percentage of cells expressing MITF as measured by immunocytochemistry (top graph) or qPCR (bottom graph) after treatment with different BMP pathway activators. FIG. 3 shows that different BMP pathway activators can be used in step (b) of the method disclosed herein.

FIG. 4A shows graphs indicating the percentage of cells expressing CRALBP as measured by immunocytochemistry under different conditions.

FIG. 4B shows a graph indicating the percentage of cells expressing MERTK as measured by immunocytochemistry under different conditions.

FIG. 4C shows graphs indicating the relative expression of RIbp1 (CRALBP) (top graph) and Mitf (bottom graph) as measured by qPCR under different conditions.

FIG. 4D shows graphs indicating the relative expression of Mertk (top graph) and Best1 (bottom graph) as measured by qPCR under different conditions.

FIG. 4E shows graphs indicating the relative expression of Silv (PMEL17) (top graph) and Tyr (bottom graph) as measured by qPCR under different conditions.

FIG. 5 shows a graph indicating the percentage of cells expressing CRALBP at D9-19 as measured by immunocytochemistry under different conditions. FIG. 5 shows that activin A is a suitable activin pathway activator for use in the method disclosed herein and that a short exposure to activin A is sufficient to induce expression of RPE markers.

FIGS. 6 and 7 show graphs indicating the percentage of cells expressing PMEL17 (top graph) and CRALBP (bottom graph) at D9-19-20 in 96 well plates (FIG. 6) and 384 well plates (FIG. 7) as measured by immunocytochemistry when cells are replated (step (e) of the early replate embodiment) at different seeding densities on different plates and cultured in media optionally comprising cAMP. FIGS. 6 and 7 show inter alia that different seeding densities can be used in step (e).

FIG. 8A shows the cells at Day 49 (step (b)) of the late replate embodiment after treatment with SMAD inhibitors, BMP pathway activator and culture in basic medium until Day 49. FIG. 8B shows the cells after 12 days of culture (step (d)) post replating. FIG. 8C shows graphs indicating the percentage of cells expressing PMEL17 (top graph) and CRALBP (bottom graph) as measured by immunocytochemistry after 15 days of culture post replating.

FIG. 9A shows a Principal Component Analysis (PCA) plot of 7 RPE samples generated by directed differentiation along with RPE cells generated by spontaneous differentiation as well as de-differentiated controls. FIG. 9B shows the loading plots used for PCA which indicates contribution of each of the genes tested to the clustering of the samples. FIG. 9C shows the comparison of whole genome transcript profiling of RPE cells obtained by Directed Differentiation (both Early and Late replating as disclosed in examples 1 and 8), RPE cells obtained by Spontaneous Differentiation and hES cells.

FIG. 10A shows a graph indicating the ratio of concentration of VEGF to concentration of PEDF in the spent media of the bottom and top chambers of the Transwell® at week 10. FIG. 10A is consistent with the conclusion that the cells obtained by the method of the invention are RPE cells.

FIG. 10B shows a graph depicting the increase of PEDF and VEGF in the spent media of cells cultured after the replating step (c). FIG. 10B is consistent with the conclusion that the cells obtained by the method of the invention are RPE cells.

FIG. 11A is a schematic representation of the Epithelial-Mesenchymal Transition and Mesenchymal-Epithelial Transition occurring during RPE cells expansion.

FIG. 11B shows a graph indicating the number of cells (Hoescht positive nuclei per frame imaged) obtained after expansion of RPE cells under different conditions. FIG. 11B shows that the use of cAMP or an agent which increases the intracellular concentration of cAMP step increases the yield of the expansion step.

FIG. 11C shows a graph indicating the percentage of cells expressing PMEL17 as measured by immunocytochemistry after expansion of RPE cells optionally in the presence of cAMP.

FIG. 11D shows a graph indicating the percentage of cells expressing PMEL17 as measured by immunocytochemistry after expansion of RPE cells optionally in the presence of an agent that increases intracellular cAMP such as Forskolin.

FIG. 11E shows a graph indicating the percentage of EdU incorporation in RPE cells expanded in the presence of cAMP.

FIG. 11F shows a graph indicating the number of cells per cm² obtained after expansion of RPE cells in the presence of cAMP.

FIG. 11G shows a graph indicating the percentage of cells expressing Ki67 at D14 as measured by immunocytochemistry after expansion of RPE cells optionally in the presence cAMP.

FIG. 11H shows a graph indicating the percentage of cells expressing PMEL17 at D14 as measured by immunocytochemistry after expansion of RPE cells optionally in the presence cAMP.

FIG. 11I shows a graph indicating the expression of Mitf at week 5 as measured by qPCR after expansion of RPE cells optionally in the presence cAMP.

FIG. 11J shows a graph indicating the expression of Silv at week 5 as measured by qPCR after expansion of RPE cells optionally in the presence cAMP.

FIG. 11K shows a graph indicating the expression of Tyr at week 5 as measured by qPCR after expansion of RPE cells optionally in the presence cAMP.

FIG. 12A shows a graph indicating the percentage of EdU incorporation in RPE cells expanded in the presence of a SMAD inhibitor.

FIG. 12B shows a graph indicating the expression of Best1 at week 5 as measured by qPCR after expansion of RPE cells optionally in the presence of a SMAD inhibitor.

FIG. 12C shows a graph indicating the expression of RIbp1 at week 5 as measured by qPCR after expansion of RPE cells optionally in the presence of a SMAD inhibitor.

FIG. 12D shows a graph indicating the expression of Grem1 at week 5 as measured by qPCR after expansion of RPE cells optionally in the presence of a SMAD inhibitor.

FIG. 13A shows a graph indicating the percentage of EdU incorporation at Day 14 in RPE cells expanded in the presence of an antibody against TGFβ1 and TGFβ2 ligands.

FIG. 13B shows a graph indicating the percentage of cells expressing PMEL17 at D14 as measured by immunocytochemistry after expansion of RPE cells optionally in the presence of an antibody against TGFβ1 and TGFβ2 ligands.

FIGS. 13C, 13D, 13E, 13F, 13G and 13H show respectively a graph indicating the percentage of cells expressing Best1, Merkt, Grem1, Silv, Lrat and Rpe65 as measured by qPCR after expansion of RPE cells optionally in the presence of an antibody against TGFβ1 and TGFβ2 ligands.

FIG. 14A shows a graph indicating the relative expression of hESC markers as measured by qPCR in cells stained with an anti-CD59 antibody triaged by flow cytometry.

FIG. 14B shows a graph indicating the relative expression of RPE markers as measured by qPCR in cells stained with an anti-CD59 antibody triaged by flow cytometry.

FIGS. 15A, 15B, 15C and 15D show respectively the percentage of cells expressing OCT4, LHX2, PAX6 and CRALBP at D2, D9 (and D9-19 for CRALBP) as measured by immunocytochemistry during the differentiation of iPSC in RPE cells.

FIGS. 15E, 15F and 15G show respectively the percentage of cells expressing Best1, Mertk and Silv as measured by qPCR after second replating (D9-19-45) in a directed differentiation protocol using iPSC as starting material. ESDD means RPE cells obtained by directed differentiation using hESC as starting material. IPSDD means RPE cells obtained by directed differentiation using iPSC as starting material.

### DETAILED DESCRIPTION

In some embodiments, the term “pluripotent cell” refers to a cell capable of differentiating to cell types of the three germ layers (e.g., can differentiate to ectodermal, mesodermal and endodermal cell types) under the appropriate conditions. Pluripotent cells can also be maintained in culture in vitro for a prolonged period of time in an undifferentiated state. In a preferred embodiment, the pluripotent cells are of vertebrate, in particular mammalian, preferably human, primate or rodent origin. Preferred pluripotent cells are human pluripotent cells. Examples of pluripotent cells are embryonic stem cells or induced pluripotent stem cells. In some embodiments, the pluripotent cells are obtained by a method which does not involve destruction of human embryos.

In some embodiments, the pluripotent cell is an embryonic stem cell (ESC).

In some embodiments, ESC refers to stem cells derived from an embryo. In some embodiments, the embryo is obtained from in vitro fertilized embryos.

In some embodiments, ESC refers to cells derived from the inner cell mass of blastocysts or morulae that have been serially passaged as cell lines. In some embodiments, said blastocysts are obtained from an in vitro fertilized embryo. In some embodiments, said blastocysts are obtained from a non-fertilized oocyte which is parthenogenetically activated to cleave and develop to the blastocyst stage.

ESC may be obtained by methods known to the skilled person (see for example U.S. Pat. No. 5,843,780, which is herein incorporated by reference in its entirety).

For example, for the isolation of hESCs from a blastocyst, the zona pellucida is removed and the inner cell mass is isolated by immunosurgery, in which the trophectoderm cells are lysed and removed from the intact inner cell mass by gentle pipetting. The inner cell mass is then plated in a tissue culture flask containing the appropriate medium which enables its outgrowth. Following 9 to 15 days, the inner cell mass derived outgrowth is dissociated into clumps either by mechanical dissociation or by enzymatic digestion and the cells are then replated on a fresh tissue culture medium. Colonies demonstrating undifferentiated morphology are individually selected by micropipette, mechanically dissociated into clumps, and replated. Resulting ESCs are then routinely split every 1-2 weeks.

In some embodiments, the term ESC refers to cells isolated from one or more blastomeres of an embryo, preferably without destroying the remainder of the embryo (see, for example US20060206953 or US20080057041, which are herein incorporated by reference in their entirety).

In a preferred embodiment, the pluripotent cell is a human embryonic stem cell. In a preferred embodiment, the pluripotent cell is a human embryonic stem cell obtained without destruction of an embryo. In a preferred embodiment, the pluripotent cell is a human embryonic stem cell originating from a well established cell line such as MA01, MA09, ACT-4, H1, H7, H9, H14, WA25, WA26, WA27, Shef-1, Shef-2, Shef-3, Shef-4 or ACT30 embryonic stem cell.

In some embodiments, ESC, regardless of their source or the particular method used to produce them, can be identified based on the: (i) ability to differentiate into cells of all three germ layers, (ii) expression of at least Oct-4 and alkaline phosphatase, and (iii) ability to produce teratomas when transplanted into immunocompromised animals.

In some embodiments, the pluripotent cell is an induced pluripotent stem cell (iPSC).

In some embodiments, an iPSC is a pluripotent cell derived from a non pluripotent cell such as for example an adult somatic cell, by reprogramming said somatic cell for example by expressing or inducing expression of a combination of factors. IPSCs are commercially available or can be obtained by methods known to the skilled person. IPSCs can be generated using for example fetal, postnatal, newborn, juvenile, or adult somatic cells. In certain embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct-4, Sox2, Nanog, and Lin28 (see for example EP2137296, which is herein incorporated by reference in its entirety). In some embodiments, the iPSCs are obtained by reprogramming a somatic cell using a combination of small molecule compounds (see for example, Science, Vol. 341 no. 6146, pp. 651-654, which is herein incorporated by reference in its entirety).

In a preferred embodiment, the pluripotent cell is a human induced pluripotent stem cell. In a preferred embodiment, the pluripotent cell is an induced pluripotent stem cell derived from a human adult somatic cell.

IPSO can be obtained for example using methods disclosed in US20090068742, US20090047263, US20090227032, US20100062533, US20130059386, WO2008118820, or WO2009006930, which are herein incorporated by reference in their entirety.

In some embodiments, the term “SMAD inhibitor” refers to an inhibitor of Small Mothers Against Decapentaplegic (SMAD) protein signaling.

In some embodiments, the term “first SMAD inhibitor” refers to an inhibitor of BMP type 1 receptor ALK2. In some embodiments, the first SMAD inhibitor is an inhibitor of BMP type 1 receptors ALK2 and ALK3. In some embodiments, the first SMAD inhibitor prevents Smad1, Smad5 and/or Smad8 phosphorylation. In some embodiments, the first SMAD inhibitor is a dorsomorphin derivative. In some embodiments, the first SMAD inhibitor is selected from dorsomorphin, noggin, chordin or 4-(6-(4-(piperazin-1-yl)phenyl)pyrazolo[1,5-a]pyrimidin-3-yl)quinoline (LDN193189). In a preferred embodiment, the first SMAD inhibitor is 4-(6-(4-(piperazin-1-yl)phenyl)pyrazolo[1,5-a]pyrimidin-3-yl)quinoline (LDN193189) or a salt or hydrate thereof.

LDN193189 is a commercially available compound of formula

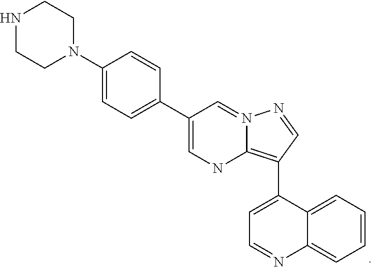

In some embodiments, the term “second SMAD inhibitor” refers to an inhibitor of transforming growth factor-β superfamily type I activin receptor-like kinase (ALK) receptors. In some embodiments, the second SMAD inhibitor is an inhibitor of ALK5. In some embodiments, the second SMAD inhibitor is an inhibitor of ALK5 and ALK4. In some embodiments, the second SMAD inhibitor is an inhibitor of ALK5 and ALK4 and ALK7. In some embodiments, the second SMAD inhibitor is 4-(4-(benzo[d][1,3]dioxol-5-yl)-5-(pyridin-2-yl)-1H-imidazol-2-yl)benzamide (SB-431542) or a salt or hydrate thereof.

SB-431542 is a commercially available compound of formula

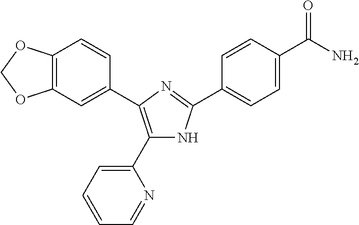 [SMILES: NC(=O)c1ccc(-c2nc(-c3ccc4c(c3)OCO4)c(-c3ccccn3)[nH]2)cc1]

In some embodiments, the second SMAD inhibitor is selected from:
4-(4-(benzo[d][1,3]dioxol-5-yl)-5-(pyridin-2-yl)-1H-imidazol-2-yl)benzamide;
2-methyl-5-(6-(m-tolyl)-1H-imidazo[1,2-a]imidazol-5-yl)-2H-benzo[d][1,2,3]triazole;
2-(6-methylpyridin-2-yl)-N-(pyridin-4-yl)quinazolin-4-amine;
2-(3-(6-methylpyridin-2-yl)-1H-pyrazol-4-yl)-1,5-naphthyridine;
4-(2-(6-methylpyridin-2-yl)-5,6-dihydro-4H-pyrrolo[1,2-b]pyrazol-3-yl)phenol;
2-(4-methyl-1-(6-methylpyridin-2-yl)-1H-pyrazol-5-yl)thieno[3,2-c]pyridine;
4-(5-(3,4-dihydroxyphenyl)-1-(2-hydroxyphenyl)-1H-pyrazol-3-yl)benzamide;
2-(5-chloro-2-fluorophenyl)-N-(pyridin-4-yl)pteridin-4-amine; or, 6-methyl-2-phenylthieno[2,3-d]pyrimidin-4(3H)-one;
or a salt or hydrate thereof.

The above compounds are commercially available or can be prepared by processes known to the skilled person (see for example Surmacz et Al, Stem Cells 2012; 30:1875-1884).

In some embodiments, the second SMAD inhibitor is selected from 3-(6-Methyl-2-pyridinyl)-N-phenyl-4-(4-quinolinyl)-1H-pyrazole-1-carbothioamide (A 83-01), 2-(5-Benzo[1,3]dioxol-5-yl-2-tert-butyl-3H-imidazol-4-yl)-6-methylpyridine (SB-505124), 7-(2-morpholinoethoxy)-4-(2-(pyridin-2-yl)-5,6-dihydro-4H-pyrrolo[1,2-b]pyrazol-3-yl)quinoline (LY2109761) or 4-[3-(2-pyridinyl)-1H-pyrazol-4-yl]-quinoline (LY364947).

In some embodiments, the BMP pathway activator comprises a BMP. In some embodiments, the BMP pathway activator comprises a BMP selected from BMP2, BMP3, BMP4, BMP6, BMP7, BMP8, BMP9, BMP10, BMP11 or BMP15. In some embodiments, the BMP pathway activator is a BMP homodimer, preferably a BMP2, BMP3, BMP4, BMP6, BMP7, BMP8, BMP9, BMP10, BMP11 or BMP15 homodimer. In some embodiments, the BMP pathway activator is a BMP homodimer, preferably a BMP2, BMP3, BMP4, BMP6, BMP7, or BMP8 homodimer. In some embodiments, the BMP pathway activator is a BMP heterodimer, preferably comprising a BMP selected from BMP2, BMP3, BMP4, BMP6, BMP7, BMP8, BMP9, BMP10, BMP11 or BMP15. In some embodiments, the BMP pathway activator is a BMP heterodimer, preferably comprising a BMP selected from BMP2, BMP3, BMP4, BMP6, BMP7 or BMP8. In some embodiments, the BMP pathway activator is a BMP2/6 heterodimer, a BMP4/7 heterodimer or a BMP3/8 heterodimer. In some embodiments, the BMP pathway activator is a BMP4/7 heterodimer.

In some embodiments, the BMP pathway activator is a small molecule activator of BMP signaling (see for example PLOS ONE, March 2013, Vol. 8 (3), e59045, which is herein incorporated by reference in its entirety).

In some embodiments, the term “Retinal Pigment Epithelial cell” or “RPE cell” refers to a cell having the morphological and functional attributes of an adult RPE cell, preferably an adult human RPE cell.

In some embodiments, the RPE cell has the morphological attributes of an adult RPE cell preferably an adult human RPE cell. In some embodiments, the RPE cell has a cobblestone morphology. In some embodiments, the RPE cell is pigmented. The shape, morphology and pigmentation of RPE cells can be observed visually.

In some embodiments, the RPE cell expresses at least one of the following RPE markers: MITF, PMEL17, CRALBP, MERTK, BEST1 and ZO-1. In some embodiments, the RPE cell expresses at least two, three, four or five of the following RPE markers: MITF, PMEL17, CRALBP, MERTK, BEST1 and ZO-1. In some embodiments, the expression of the RPE markers is measured by immunocytochemistry. In some embodiments, the expression of the RPE markers is measured by immunocytochemistry as detailed in the example section. In some embodiments, the expression of markers is measured by quantitative PCR. In some embodiments, the expression of the RPE markers is measured by quantitative PCR as detailed in the example section.

In some embodiments, the RPE cell does not express Oct4

In some embodiments, the RPE cell has the functional attributes of an adult RPE cell, preferably an adult human RPE cell. In some embodiments, the RPE cell secretes VEGF. In some embodiments, the RPE cell secretes PEDF. In some embodiments, the RPE cell secretes PEDF and VEGF. In some embodiments, VEGF and/or PEDF secretion by RPE cells is measured by a quantitative immunoassay. In some embodiments, VEGF and/or PEDF secretion by RPE cells is measured as disclosed in the examples.

In a preferred embodiment, the RPE cell has a cobblestone morphology, is pigmented and expresses at least one of MITF, PMEL17, CRALBP, MERTK, BEST1 and ZO-1. In a preferred embodiment, the RPE cell has a cobblestone morphology, is pigmented and expresses at least two of MITF, PMEL17, CRALBP, MERTK, BEST1 and ZO-1. In a preferred embodiment, the RPE cell has cobblestone morphology, is pigmented, expresses at least two of MITF, PMEL17, CRALBP, MERTK, BEST1 and ZO-1 and secretes VEGF and PEDF.

When a parameter is defined as “between a low value and high value”, such low and high value should be considered as part of the defined range.

### Early Replating

In one embodiment (early replating embodiment), the invention relates to a method for producing RPE cells comprising the following steps:

(a) culturing pluripotent cells in the presence of a first SMAD inhibitor and a second SMAD inhibitor;

(b) culturing the cells of step (a) in the presence of a BMP pathway activator and in the absence of the first and second SMAD inhibitors; and,

(c) replating the cells of step (b).

In some embodiments, in step (a), the pluripotent cells are cultured for at least 1 day. In some embodiments, in step (a), the pluripotent cells are cultured for at least 1 day, at least 2 days, at least 3 days or at least 4 days. In some embodiments, in step (a), the pluripotent cells are cultured for between 2 and 10 days. In some embodiments, in step (a), the pluripotent cells are cultured for between 2 and 6 days. In some embodiments, in step (a), the pluripotent cells are cultured for between 3 and 5 days. In some embodiments, in step (a), the pluripotent cells are cultured for about 4 days.

In some embodiments, in step (a), the concentration of first SMAD inhibitor is between 0.5 nM and 10 μM. In some embodiments, in step (a), the concentration of first SMAD inhibitor is between 1 nM and 5 μM. In some embodiments, in step (a), the concentration of first SMAD inhibitor is between 1 nM and 2 μM. In some embodiments, in step (a), the concentration of first SMAD inhibitor is between 500 nM and 2 μM. In some embodiments, in step (a), the concentration of first SMAD inhibitor is about 1 μM. In a preferred embodiment, the first SMAD inhibitor is LDN193189.

In some embodiments, in step (a), the concentration of second SMAD inhibitor is between 0.5 nM and 100 μM. In some embodiments, in step (a), the concentration of second SMAD inhibitor is between 100 nM and 50 μM. In some embodiments, in step (a), the concentration of second SMAD inhibitor is between 1 μM and 50 μM. In some embodiments, in step (a), the concentration of second SMAD inhibitor is between 5 μM and 20 μM. In some embodiments, in step (a), the concentration of second SMAD inhibitor is at least 5 μM. In some embodiments, in step (a), the concentration of second SMAD inhibitor is about 10 μM. In a preferred embodiment, the second SMAD inhibitor is SB-431542.

In some embodiments, in step (b), the concentration of BMP pathway activator is between 1 ng/mL and 10 μg/mL. In some embodiments, in step (b), the concentration of BMP pathway activator is between 5 ng/mL and 1 μg/mL. In some embodiments, in step (b), the concentration of BMP pathway activator is between 50 ng/mL and 500 ng/mL. In some embodiments, in step (b), the concentration of BMP pathway activator is about 100 ng/mL. In a preferred embodiment the BMP pathway activator is a BMP4/7 heterodimer.

In some embodiments, in step (b), the cells are cultured for at least 1 day. In some embodiments, in step (b), the cells are cultured for at least 1 day, at least 2 days, at least 3 days or at least 4 days. In some embodiments, in step (b), the cells are cultured for at least 3 days. In some embodiments, in step (b), the cells are cultured for between 2 and 20 days. In some embodiments, in step (b), the cells are cultured for between 2 and 10 days. In some embodiments, in step (b), the cells are cultured for between 2 and 6 days. In some embodiments, in step (b), the cells are cultured for between 2 and 4 days. In some embodiments, in step (b), the cells are cultured for about 3 days.

In some embodiments, before step (a), the cells are cultured as a monolayer at an initial density of at least 20000 cells/cm². In some embodiments, before step (a), the cells are cultured as a monolayer at an initial density of at least 100000 cells/cm². In some embodiments, before step (a), the cells are cultured as a monolayer at an initial density of between 20000 and 1000000 cells/cm². In some embodiments, before step (a), the cells are cultured as a monolayer at an initial density of between 100000 and 500000 cells/cm². In some embodiments, before step (a), the cells are cultured as a monolayer at an initial density of about 240000 cells/cm².

In some embodiments, in step (c), the cells are replated at a density of at least 1000 cells/cm². In some embodiments, in step (c), the cells are replated at a density of at least 10000 cells/cm². In some embodiments, in step (c), the cells are replated at a density of at least 20000 cells/cm². In some embodiments, in step (c), the cells are replated at a density of at least 100000 cells/cm². In some embodiments, in step (c), the cells are replated at a density of between 20000 and 5000000 cells/cm². In some embodiments, in step (c), the cells are replated at a density of between 100000 and 1000000 cells/cm². In some embodiments, in step (c), the cells replated at a density of about 500000 cells/cm². In some embodiments, in step (c), the cells are replated on fibronectin, Matrigel® or Cellstart®.

In some embodiments, the invention relates to a method for producing RPE cells comprising steps (a), (b) and (c) disclosed above and further comprising the following steps:

(d) culturing the replated cells of step (c) in the presence of an activin pathway activator;

(e) replating the cells of step (d); and,

(f) culturing the replated cells of step (e).

In some embodiments, the activin pathway activator is activin A pathway activator. In some embodiments, the activin pathway activator comprises activin A or activin B. In a preferred embodiment, the activin pathway activator is activin A.

In some embodiments, in step (d), the cells are cultured in the presence of activin pathway activator for at least 1 day. In some embodiments, in step (d), the cells are cultured in the presence of activin pathway activator for at least 3 days. In some embodiments, in step (d), the cells are cultured in the presence of activin pathway activator for between 1 and 50 days, 3 and 30 days or 3 and 20 days.

In some embodiments, in step (d), the cells are cultured in the presence of activin pathway activator for at least 1 day and the cells are further cultured without the activin pathway activator for at least 3 days. In some embodiments, in step (d), the cells are cultured in the presence of activin pathway activator for at least 3 days and the cells are further cultured without the activin pathway activator for at least 4 days. In some embodiments, in step (d), the cells are cultured in the presence of activin pathway activator for between 1 and 10 days and the cell are further cultured without the activin pathway activator for between 5 and 30 days. In some embodiments, in step (d), the cells are cultured in the presence of activin pathway activator for about 3 days and the cell are further cultured without the activin pathway activator for between 5 and 30 days.

In some embodiments, in step (d), the concentration of activin pathway activator is between 1 ng/mL and 10 μg/mL. In some embodiments, in step (d), the concentration of activin pathway activator is between 1 ng/mL and 1 μg/mL. In some embodiments, in step (d), the concentration of activin pathway activator is between 10 ng/mL and 500 ng/mL. In some embodiments, in step (d), the activin pathway activator is activin A at a concentration of about 100 ng/mL.

In some embodiments, in step (e), the cells are replated at a density of at least 1000 cells/cm². In some embodiments, in step (e), the cells are replated at a density of at least 20000 cells/cm². In some embodiments, in step (e), the cells are replated at a density of at least 100000 cells/cm². In some embodiments, in step (e), the cells are replated at a density of between 20000 and 5000000 cells/cm². In some embodiments, in step (e), the cells are replated at a density of between 20000 and 1000000 cells/cm². In some embodiments, in step (e), the cells are replated at a density of between 20000 and 500000 cells/cm². In some embodiments, in step (e), the cells are replated at a density of about 200000 cells/cm². In some embodiments, in step (e), the cells are replated on fibronectin, Matrigel® or Cellstart®.

In some embodiments, in step (f), the cells are cultured for at least 5 days. In some embodiments, in step (f), the cells are cultured for at least 7 days, at least 14 days or at least 21 days. In some embodiments, in step (f), the cells are cultured for at least 14 days. In some embodiments, in step (f), the cells are cultured for between 5 and 40 days. In some embodiments, in step (f), the cells are cultured for between 10 and 35 days. In some embodiments, in step (f), the cells are cultured for between 21 and 35 days. In some embodiments, in step (f), the cells are cultured for about 28 days.

In some embodiments, in step (d), the cells are cultured in the presence of cAMP, preferably at a concentration between 0.01 mM to 1M. In some embodiments, in step (d), the cells are cultured in the presence of 0.1 mM to 5 mM cAMP. In some embodiments, in step (d), the cells are cultured in the presence of 0.5 mM cAMP.

In some embodiments, in step (f), the cells are cultured in the presence of cAMP, preferably at a concentration between 0.01 mM to 1M. In some embodiments, in step (f), the cells are cultured in the presence of 0.1 mM to 5 mM cAMP. In some embodiments, in step (f), the cells are cultured in the presence of 0.5 mM cAMP.

The present disclosure also includes methods where the above disclosed embodiments of steps (a), (b), (c), (d), (e) and/or (f) are combined.

In a preferred embodiment, the invention relates to a method for producing retinal pigment epithelial cells comprising the following steps:

(a) culturing human ESCs or human iPSCs in the presence of 500 nM to 2 μM LDN193189 and 5 μM to 20 μM SB-431542 for between 3 and 5 days;

(b) culturing the cells of step (a) in the presence of 50 ng/mL to 500 ng/mL of BMP2/6 heterodimer, BMP4/7 heterodimer or BMP3/8 heterodimer and in the absence of LDN193189 and SB-431542 for between 2 and 6 days; and,

(c) replating the cells of step (b) at a density of between 100000 and 1000000 cells/cm².

(d) culturing the replated cells of step (c) in the presence of about 10 ng/mL to 500 ng/mL activin A for between 3 and 30 days;

(e) replating the cells of step (d) at a density of between 20000 and 500000 cells/cm²; and,

(f) culturing the replated cells of step (e) for between 10 and 35 days.

### Late Replating

In an alternative embodiment (late replating embodiment), the method for producing RPE cells comprises the following steps:

(a) culturing pluripotent cells in the presence of a first SMAD inhibitor and a second SMAD inhibitor;

(b) culturing the cells of step (a) in the presence of a BMP pathway activator and in the absence of the first and second SMAD inhibitors; and then, culturing said cells for at least 10 days in the absence of the BMP pathway activator;

(c) replating the cells of step (b) having a cobblestone morphology; and,

(d) culturing the replated cells of step (c).

The embodiments disclosed above in connection with steps (a), (b) and (c) of the early replating embodiment are also embodiments of steps (a), (b) and (c) of the late replating embodiment.

In some embodiments, in step (b), the cells are cultured for at least 20 days in the absence of the BMP pathway activator. In some embodiments, in step (b), the cells are cultured for at least 30 days in the absence of the BMP pathway activator. In some embodiments, in step (b), the cells are cultured for at least 40 days in the absence of the BMP pathway activator. In some embodiments, in step (b), the cells are cultured for between 10 and 60 days in the absence of the BMP pathway activator. In some embodiments, in step (b), the cells are cultured for between 30 and 50 days in the absence of the BMP pathway activator. In some embodiments, in step (b), the cells are cultured for about 40 days in the absence of the BMP pathway activator.

In some embodiments, in step (c), the cells are replated at a density of at least 1000 cells/cm². In some embodiments, in step (c), the cells are replated at a density of at least 20000 cells/cm². In some embodiments, in step (c), the cells are replated at a density of at least 100000 cells/cm². In some embodiments, in step (c), the cells are replated at a density of between 20000 and 5000000 cells/cm². In some embodiments, in step (c), the cells are replated at a density of between 50000 and 1000000 cells/cm². In some embodiments, in step (c), the cells are replated at a density of between 50000 and 500000 cells/cm². In some embodiments, in step (c), the cells are replated at a density of about 200000 cells/cm².

In some embodiments, in step (d), the cells are cultured for at least 3 days. In some embodiments, in step (d), the cells are cultured for at least 5 days. In some embodiments, in step (d), the cells are cultured for at least 10 days. In some embodiments, in step (d), the cells are cultured for at least 14 days. In some embodiments, in step (d), the cells are cultured for between 10 and 40 days. In some embodiments, in step (d), the cells are cultured for between 10 and 20 days. In some embodiments, in step (d), the cells are cultured for about 14 days.

In some embodiments, in step (d), the cells are cultured in the presence of cAMP, preferably at a concentration between 0.01 mM to 1M. In some embodiments, in step (d), the cells are cultured in the presence of 0.1 mM to 5 mM cAMP. In some embodiments, in step (d), the cells are cultured in the presence of 0.5 mM cAMP.

In some embodiments, the method further comprises the following additional steps:

(e) replating the cells of step (d);

(f) culturing the replated cells of step (e).

In some embodiments, in step (e), the cells are replated at a density of at least 1000 cells/cm². In some embodiments, in step (e), the cells are replated at a density of at least 20000 cells/cm². In some embodiments, in step (e), the cells are replated at a density of at least 100000 cells/cm². In some embodiments, in step (e), the cells are replated at a density of between 20000 and 5000000 cells/cm². In some embodiments, in step (e), the cells are replated at a density of between 50000 and 1000000 cells/cm². In some embodiments, in step (e), the cells are replated at a density of between 50000 and 500000 cells/cm². In some embodiments, in step (e), the cells replated at a density of about 200000 cells/cm².

In some embodiments, in step (f), the cells are cultured for at least 10 days. In some embodiments, in step (f), the cells are cultured for at least 14 days. In some embodiments, in step (f), the cells are cultured for at least 20 days. In some embodiments, in step (f), the cells are cultured for at least 25 days. In some embodiments, in step (f), the cells are cultured for at least 40 days. In some embodiments, in step (f), the cells are cultured for between 10 and 60 days. In some embodiments, in step (f), the cells are cultured for between 15 and 40 days. In some embodiments, in step (f), the cells are cultured for about 28 days.

The present disclosure also includes methods where the above disclosed embodiments of steps (a), (b), (c), (d), (e) and/or (f) are combined.

In a preferred embodiment, the invention relates to a method for producing RPE cells comprising the following steps:

(a) culturing human ESCs or human iPSCs in the presence of 500 nM to 2 μM LDN193189 and 5 μM to 20 μM SB-431542 for between 3 and 5 days;

(b) culturing the cells of step (a) in the presence of 50 ng/mL to 500 ng/mL of BMP2/6 heterodimer, BMP4/7 heterodimer or BMP3/8 heterodimer and in the absence of LDN193189 and SB-431542 for between 2 and 6 days; and then, culturing said cells for between 30 and 50 days in the absence of the BMP pathway activator

(c) replating the cells of step (b) having a cobblestone morphology at a density of between 50000 and 500000 cells/cm²; and,

(d) culturing the replated cells of step (c) for between 10 and 20 days;

(e) replating the cells of step (d) at a density of between 50000 and 500000 cells/cm²; and,

(f) culturing the replated cells of step (e) for between 15 and 40 days.

The RPE cells prepared by the methods disclosed herein (including early replating and late replating) can be harvested by various methods known to the skilled person. For example, the RPE cells can be harvested by mechanical dissection or by dissociation with an enzyme such as papain or trypsin.

The RPE cells prepared by the methods disclosed herein can be further purified, for example without limitation, by techniques such as Fluorescence Activated Cell Sorting (FACS) or Magnetic Activated Cell Sorting (MACS). These techniques involve the use of antibodies against RPE-specific cell surface proteins (positive selection). In a preferred embodiment, said RPE specific cell surface protein is CD59. For FACS, RPE cells can be labelled with fluorophore conjugated antibodies targeting specific RPE cell surface markers. These labelled cells can be purified using a cytometer to give rise to a highly homogeneous and purified RPE population free of any contaminating cell type. Similarly in MACS, RPE cells can be labelled with antibodies conjugated to magnetic nanoparticles and further purified by application of magnetic field. Negative selection can also be applied by using antibodies targeting potential contaminating cell types which would lead to their removal and also contribute to generation of pure RPE population.

In some embodiments, the method for producing RPE cells disclosed herein comprises a purification step for enriching the cell population in cells expressing CD59. Enriching the cell population in cells expressing CD59 is a means to enrich for mature RPE cells and remove residual contaminating cells such as pluripotent cells and/or RPE progenitors that may possibly be present in the final RPE cell population.

In some embodiments, the method for producing RPE cells disclosed herein comprises a purification step comprising:
contacting the cells with an anti-CD59 antibody conjugated to a fluorophore, and,
selecting the cells that bind to the anti-CD59 antibody using FACS.

In a preferred embodiment, the anti-CD59 antibody is antibody Cat#560747 (BD Biosciences).

In some embodiments, the method for producing RPE cells disclosed herein comprises a purification step as disclosed in Example 13b.

In some embodiments, the method for producing RPE cells disclosed herein comprises a purification step comprising:
contacting the cells with an anti-CD59 antibody conjugated to a magnetic particle, and,
selecting the cells that bind to the anti-CD59 antibody using MACS.

Commercially available anti-CD59 antibody such as for example antibody Cat#560747 (BD Biosciences) can be used in the present invention.

In some embodiments, a purification step as disclosed above is performed after step (e) of the early replating method. In some embodiments, a purification step as disclosed above is performed after step (f) of the early replating method. In some embodiments, a purification step as disclosed above is performed after step (c) of the late replating method. In some embodiments, a purification step as disclosed above is performed after step (d) of the late replating method.

In some embodiments, the invention relates to a method for producing RPE cells comprising:

a) providing a population of pluripotent cells;

b) inducing the differentiation of pluripotent cells into RPE cells, and,

c) enriching the cell population for cells expressing CD59.

In some embodiments, the invention relates to a method for producing RPE cells comprising:

a) providing a population of pluripotent cells;

b) inducing the differentiation of pluripotent cells into RPE cells, and,

c) enriching the cell population for cells expressing CD59 by
contacting the cells with an anti-CD59 antibody conjugated to a fluorophore, and,
selecting the cells that bind to the anti-CD59 antibody using FACS.

In some embodiments, the invention relates to a method for producing RPE cells comprising:

a) providing a population of pluripotent cells;

b) inducing the differentiation of pluripotent cells into RPE cells, and,

c) enriching the cell population for cells expressing CD59 by
contacting the cells with an anti-CD59 antibody conjugated to a magnetic particle, and,
selecting the cells that bind to the anti-CD59 antibody using MACS.

In step b, the differentiation of pluripotent cells in RPE cells can be performed according to any method known to the skilled person such as for example spontaneous differentiation or directed differentiation methods. In particular, in step b, the differentiation of pluripotent cells into RPE cells can be performed according to any method disclosed in WO08/129554, WO09/051671, WO2011/063005, US2011269173, US20130196369, WO2013/184809, WO08/087917, WO2011/028524 or WO2014/121077, which are incorporated herein by reference.

In some embodiments, the invention relates to a method for purifying RPE cells comprising:

a) providing a cell population comprising RPE cells and non RPE cells;

b) increasing the percentage of RPE cells in the cell population by enriching the cell population for cells expressing CD59.

In some embodiments, the invention relates to a method for purifying RPE cells comprising:

a) providing a cell population comprising RPE cells and non RPE cells;

b) increasing the percentage of RPE cells in the cell population by
contacting the cell population with an anti-CD59 antibody conjugated to a fluorophore, and,
selecting the cells that bind to the anti-CD59 antibody using FACS.

In some embodiment, the invention relates to a method for purifying RPE cells comprising:

a) providing a cell population comprising RPE cells and non RPE cells;

b) increasing the percentage of RPE cells in the cell population by
contacting the cell population with an anti-CD59 antibody conjugated to a magnetic particle, and,
selecting the cells that bind to the anti-CD59 antibody using MACS.

In some embodiments, non RPE cells are pluripotent cells or RPE progenitors.

In some embodiments, the term “RPE progenitors” refers to cells derived from pluripotent cells such as hESC induced to differentiate into RPE cells but which have not fully completed the differentiation process. In some embodiments, such “RPE progenitor” comprises one or more morphological and functional attributes of an adult RPE cell and lacks at least one morphological and functional attributes of an adult RPE cells. In some embodiment, the RPE progenitor expresses one or more of OCT4, NANOG or LIN28.

In some embodiments of the methods disclosed herein, the cells are cultured in a two-dimensional culture under adhesion conditions, such as, for example, plate culture. In a preferred embodiment, the cells are cultured as a monolayer. In some embodiments, the cells are cultured on a cell-supporting substance, such as, for example without limitation, collagen, gelatin, poly-L-lysine, poly-D-lysine, laminin, fibronectin, vitronectin, Cellstart®, BME Pathclear®, or Matrigel® (Becton, Dickinson and Company). In some embodiments, the cells are cultured as a monolayer, for example, on collagen, gelatin, poly-L-lysine, poly-D-lysine, laminin, fibronectin, vitronectin, Cellstart®, BME Pathclear®, or Matrigel®. In a preferred embodiment, the cells are cultured as a monolayer on Matrigel®. In a preferred embodiment, the cells are cultured as a monolayer on fibronectin or vitronectin.

In some embodiments, some steps of the methods disclosed herein may be performed in a three-dimensional culture under non-adhesion conditions, such as suspension culture. In suspension culture, a majority of cells freely float as single cells, cell clusters and or as cell aggregates in a liquid medium. The cells can be cultured in a three dimensional system according to method known to the skilled person (see for example Keller et al, Current Opinion in Cell Biology, Vol 7 (6), 862-869 (1995)) or Watanabe et al., Nature Neuroscience 8, 288-296 (2005)).

In some embodiments, some steps of the methods disclosed herein are carried out in a three dimensional culture such as, for example without limitation, suspension culture and some steps are carried out in a two dimensional culture (e.g. cells cultured as a monolayer). In some embodiments, step (a) and/or (b) are carried out in a suspension culture and the following steps are carried out in a two dimensional culture (e.g. cells cultured as a monolayer).

In some embodiments, the cells are incubated with a Rho-associated protein kinase (ROCK) inhibitor before being plated. In some embodiments, the cells are incubated with a ROCK inhibitor before step (a). The ROCK inhibitor is a substance permitting survival of dissociated human embryonic stem cells (see K. Watanabe et Al., Nat. Biotech., 25: 681-686 (2007)). Examples of ROCK inhibitors which can be used in the method of the invention are, without limitation, Y-27632, H-1152, Y-30141, Wf-536, HA-1077, GSK269962A and SB-772077-B. In some embodiments, the ROCK inhibitor is Y-27632. In some embodiments, before step (a), the pluripotent cells are plated in the presence of a ROCK inhibitor. In some embodiments, the cells are cultured in the presence of a ROCK inhibitor for 1 or 2 days post plating. In some embodiments, the first replating of the method of the invention is carried out in the presence of a ROCK inhibitor. In some embodiments, the cells are cultured in the presence of a ROCK inhibitor for 1 or 2 days post first replating.

In the methods of the invention, the cell can be cultured in any basic medium suitable for the culture of pluripotent cells, preferably human pluripotent cells. In some embodiments, the cells are cultured in a basic medium suitable for the culture of human embryonic stem cells.

Examples of suitable basic media include, without limitation, IMDM medium, medium 199, Eagle's Minimum Essential Medium (EMEM), AMEM medium, Dulbecco's modified Eagle's Medium (DMEM), KO-DMEM, Ham's F12 medium, RPMI 1640 medium, Fischer's medium, Glasgow MEM, TesR1, TesR2, Essential 8 and mixtures thereof. In some embodiments the medium comprises serum. In some embodiments, the medium is serum free. In a preferred embodiment, the basic medium is TesR1 or TesR2.

The medium may further contain, if desirable, one or more serum substitutes, such as for example albumin, transferrin, Knockout Serum Replacement (KSR), fatty acid, insulin, a collagen precursor, trace elements, 2-mercaptoethanol, 3′-thiol, glycerol, B27-supplement, and N2-supplement, as well as one or more substances such as, lipids, amino acids, nonessential amino acids, vitamins, growth factors, cytokines, antibiotics, antioxidants, pyruvate, a buffering agent, and inorganic salts.

The basic medium used for the cell culture in the method of the invention can be supplemented as appropriate with, for example without limitation, SMAD inhibitors, BMP pathway activators, activin pathway activators and/or cAMP.

In some embodiments of the above disclosed methods, the cells used in step (a) are hESC or human IPSc and the method is carried out under xeno-free conditions, i.e without using any animal derived material other than human. For example, when the method is carried out under xeno-free conditions, the medium and the cell supporting substance do not comprise any animal derived material other than human.

In some embodiments, replating comprises dissociating the plated cells, preferably dissociating the monolayer of cells, and plating the dissociated cells. Preferably, the cells are dissociated using an enzyme such as for example trypsin, collagenase IV, collagenase I, dispase or a commercially available cell dissociation buffer. Preferably, the cells are dissociated using TrypLE Select®.

In some embodiments, the RPE cells obtained or obtainable by the methods disclosed herein are further expanded. In some embodiments the expansion step is carried out in a two dimensional culture, under adhesion conditions. In some embodiments, the expansion step comprises:
replating RPE cells; and,
culturing the replated RPE cells.

In some embodiments, the RPE cells are replated on a cell supporting substance. Suitable cell supporting substances include, for example without limitation, collagen, gelatin, poly-L-lysine, poly-D-lysine, laminin, fibronectin, vitronectin, Cellstart®, Matrigel® or BME Pathclear® (BME PathClear® is a soluble form of basement membrane purified from Engelbreth-Holm-Swarm (EHS) tumor. It is mainly comprised of laminin, collagen IV, entactin, and heparin sulfate proteoglycan). In a preferred embodiment, the cell supporting substance is selected from Matrigel®, Fibronectin or Cellstart®, preferably Cellstart®.

In some embodiments, the RPE cells are replated at a density between 1000 and 100000 cells/cm². In some embodiments, the RPE cells are replated at a density between 5000 and 100000 cells/cm². In some embodiments, the RPE cells are replated at a density between 10000 and 40000 cells/cm². In some embodiments, the RPE cells are replated at a density between 10000 and 30000 cells/cm². In some embodiments, the RPE cells are replated at a density of about 20000 cells/cm².

In some embodiments, the replated cells are cultured for at least 7 days. In some embodiments, the replated cells are cultured for at least 14 days. In some embodiments, the replated cells are cultured for at least 28 days. In some embodiments, the replated cells are cultured for at least 42 days. In some embodiments, the replated cells are cultured for between 21 days and 70 days. In some embodiments, the replated cells are cultured for between 30 days and 60 days. In some embodiments, the replated cells are cultured for about 49 days.

In some embodiments, RPE cells are cultured in the presence of cAMP, preferably at a concentration between 0.01 mM to 1M. In some embodiments, RPE cells are cultured in the presence of 0.1 mM to 5 mM cAMP. In some embodiments, RPE cells are cultured in the presence of about 0.5 mM cAMP.

In some embodiments, RPE cells are cultured in the presence of an agent which increases the intracellular concentration of cAMP. In some embodiments, said agent is an Adenyl Cyclase activator, preferably forskolin. In some embodiments, said agent is a phosphodiesterase (PDE) inhibitor, preferably a PDE1, PDE2, PDE3, PDE4, PDE7, PDE8, PDE10 and/or PDE11 inhibitor. In some embodiments, said agent is a PDE4, PDE7 and/or PDE8 inhibitor.

In some embodiments, RPE cells are cultured in the presence of a SMAD inhibitor, preferably at a concentration between 1 nM to 100 μM. In some embodiments, RPE cells are cultured in the presence of 10 nM to 10 μM SMAD inhibitor. In some embodiments, RPE cells are cultured in the presence of about 10 nM to 1 μM SMAD inhibitor. In some embodiments, said SMAD inhibitor is an inhibitor of TGFβ type I receptor (ALK5) and/or TGFβ type II receptor. In a preferred embodiment, said SMAD inhibitor is an ALK5 inhibitor. In some embodiments, said inhibitor is 2-(6-methylpyridin-2-yl)-N-(pyridin-4-yl)quinazolin-4-amine, 6-(1-(6-methylpyridin-2-yl)-1H-pyrazol-5-yl)quinazolin-4(3H)-one, or 4-methoxy-6-(3-(6-methylpyridin-2-yl)-1H-pyrazol-4-yl)quinoline. Examples of SMAD inhibitors that can be used in the present invention can also be found for example in EP2409708A1 or in Yingling J M et al. Nature Reviews/Drug Discovery Vol. 3:1011-1022 (2004).

In some embodiments, RPE cells are cultured in the presence of cAMP or an agent which increases the intracellular concentration of cAMP, preferably cAMP, and the yield of the expansion step is increased as compared to similar conditions without said agent or cAMP.

The invention also relates to a method for expanding RPE cells comprising the step of culturing said RPE cells in the presence of SMAD inhibitor, cAMP or an agent which increases the intracellular concentration of cAMP. In some embodiments, the invention relates to a method for expanding RPE cells comprising the following steps:

(a) plating RPE cells at a density of at least 1000 cells/cm², and,

(b) culturing said RPE cells in the presence of SMAD inhibitor, cAMP or an agent which increases the intracellular concentration of cAMP.

In some embodiments, in step (a), the RPE cells are plated on a cell supporting substance for example selected from collagen, gelatin, poly-L-lysine, poly-D-lysine, laminin, fibronectin, vitronectin Cellstart®, Matrige® or BME Pathclear®. In a preferred embodiment, in step (a), the cell supporting substance is selected from Matrigel®, Fibronectin or Cellstart®, preferably Cellstart®.

In some embodiments, in step (a), the RPE cells are plated at a density between 1000 and 100000 cells/cm². In some embodiments, in step (a), the RPE cells are plated at a density between 5000 and 100000 cells/cm². In some embodiments, in step (a), the RPE cells are plated at a density between 10000 and 40000 cells/cm². In some embodiments, in step (a), the RPE cells are plated at a density between 10000 and 30000 cells/cm². In some embodiments, in step (a), the RPE cells are plated at a density of about 20000 cells/cm².

In some embodiments, in step (b), the RPE cells are cultured for at least 7 days. In some embodiments, the replated cells are cultured for at least 14 days. In some embodiments, in step (b), the replated cells are cultured for at least 28 days. In some embodiments, in step (b), the replated cells are cultured for at least 42 days. In some embodiments, in step (b), the replated cells are cultured for between 21 days and 70 days. In some embodiments, in step (b) the replated cells are cultured for between 30 days and 60 days. In some embodiments the replated cells are cultured for about 49 days.

In some embodiments, in step (b), RPE cells are cultured in the presence of an agent which increases the intracellular concentration of cAMP. In some embodiments, said agent is an Adenyl Cyclase activator, preferably forskolin. In some embodiments, said agent is a phosphodiesterase (PDE) inhibitor, preferably a PDE1, PDE2, PDE3, PDE4, PDE7, PDE8, PDE10 and/or PDE11 inhibitor. In some embodiments, said agent is a PDE4, PDE7 and/or PDE8 inhibitor.

In some embodiments, in step (b), RPE cells are cultured in the presence of cAMP, preferably at a concentration between 0.01 mM to 1M. In some embodiments, in step (b), RPE cells are cultured in the presence of 0.1 mM to 5 mM cAMP. In some embodiments, in step (b), RPE cells are cultured in the presence of about 0.5 mM cAMP.

In some embodiments, in step (b), RPE cells are cultured in the presence of cAMP or an agent which increases the intracellular concentration of cAMP, preferably cAMP, and the yield of the method for expanding RPE cells is increased as compared to the same method without said agent or cAMP.

In some embodiments, in step (b), RPE cells are cultured in the presence of a SMAD inhibitor, preferably at a concentration between 1 nM to 100 μM. In some embodiments, RPE cells are cultured in the presence of 10 nM to 10 μM SMAD inhibitor. In some embodiments, RPE cells are cultured in the presence of about 10 nM to 1 μM SMAD inhibitor. In some embodiments, said SMAD inhibitor is an inhibitor of TGFβ type I receptor (ALK5) and/or TGFβ type II receptor. In a preferred embodiment, said SMAD inhibitor is an ALK5 inhibitor. In some embodiments, said inhibitor is 2-(6-methylpyridin-2-yl)-N-(pyridin-4-yl)quinazolin-4-amine, 6-(1-(6-methylpyridin-2-yl)-1H-pyrazol-5-yl)quinazolin-4(3H)-one, or 4-methoxy-6-(3-(6-methylpyridin-2-yl)-1H-pyrazol-4-yl)quinoline. Examples of SMAD inhibitor that can be used in the present invention can also be found for example in EP2409708A1 or in Yingling J M et al. Nature Reviews/Drug Discovery Vol. 3:1011-1022 (2004).

In some embodiments, the invention relates to RPE cells obtained by a method disclosed herein. In some embodiments, the invention relates to RPE cells obtainable by a method disclosed herein.

The RPE cells obtained or obtainable by the methods disclosed herein can be used as a research tool. For example, the RPE cells can be used in in vitro models for the development of new drugs to promote their survival, regeneration and/or function or for high throughput screening for compounds that have a toxic or regenerative effect on RPE cells.

The RPE cells obtained or obtainable by the methods disclosed herein can be used in therapy. In some embodiments, the RPE cells can be used for the treatment of retinal diseases.

In some embodiments, the RPE cells are formulated in a pharmaceutical composition suitable for transplantation into the eye of a subject affected with a retinal disease.

In some embodiments, the pharmaceutical composition suitable for transplantation into the eye comprises a structure suitable for supporting RPE cells and RPE cells. Non limitative examples of such pharmaceutical compositions are disclosed in WO2009/127809, WO2004/033635 or WO2012/009377 or WO2012177968, which are herein incorporated by reference in their entirety.

In a preferred embodiment the pharmaceutical composition comprises a porous membrane and RPE cells. In some embodiments, the pores of the membrane are between 0.2 μm and 0.5 μm in diameter and the pore density is between 110⁷ and 3×10⁸ pores per cm². In some embodiments the membrane is coated on one side with a coating supporting RPE cells. In some embodiments, the coating comprises a glycoprotein, preferably selected from laminin or vitronectin. In a preferred embodiment, the coating comprises vitronectin. In some embodiments, the membrane is made of polyester.

In an alternative embodiment, the pharmaceutical composition comprises RPE cells in suspension in a medium suitable for transplantation into the eye of the subject. Examples of such pharmaceutical compositions are disclosed in WO2013/074681, which is herein incorporated by reference in its entirety.

The RPE cells obtained by the method disclosed herein may be transplanted to various target sites within a subject's eye. In accordance with one embodiment, the transplantation of the RPE cells is to the subretinal space of the eye (between the photoreceptor outer segments and the choroids). In addition, transplantation into additional ocular compartments can be considered including the vitreal space, the inner or outer retina, the retinal periphery and within the choroids.

Transplantation of RPE cells into the eye can be performed by various techniques known in the art (see for example U.S. Pat. Nos. 5,962,027, 6,045,791 and 5,941,250, which are herein incorporated by reference in their entirety).

In some embodiments, transplantation is performed via pars plana vitrectomy surgery followed by delivery of the cells through a small retinal opening into the sub-retinal space. In some embodiments, the RPE cells are transplanted into the eye using a suitable device (see for example WO2012/099873 or WO2012/004592, which are herein incorporated by reference in their entirety).

In some embodiments, the transplantation is performed by direct injection into the eye of the subject.

In some embodiments, the RPE cells obtained by the methods disclosed herein can be used for the treatment of retinal diseases. In some embodiments, the invention relates to RPE cells obtained or obtainable by the methods disclosed herein or a pharmaceutical composition comprising such cells for use in the treatment of retinal disease in a subject. In some embodiments, the invention relates to the use of RPE cells obtained or obtainable by the methods disclosed herein or a pharmaceutical composition comprising such cells for the manufacture of a medicament for the treatment of retinal disease in a subject. In some embodiments, the invention relates to a method for the treatment of a retinal disease in a subject by administering RPE cells obtained or obtainable by the methods disclosed herein or a pharmaceutical composition comprising such cells to said subject.

In some embodiments, the subject is a mammal, preferably a human.

In some embodiments, the retinal disease is a disease associated with retinal dysfunction, retinal injury, and/or loss or degradation of retinal pigment epithelium. In some embodiments, the retinal disease is selected from retinitis pigmentosa, leber's congenital amaurosis, hereditary or acquired macular degeneration, age related macular degeneration (AMD), Best disease, retinal detachment, gyrate atrophy, choroideremia, pattern dystrophy as well as other dystrophies of the RPE cells, diabetic retinopathy or Stargardt disease. In a preferred embodiment, retinal disease is retinitis pigmentosa or age related macular degeneration (AMD). In a preferred embodiment, the retinal disease is age related macular degeneration.

### EXAMPLES

### Example 1

### Directed Differentiation with Early Replating

All work was carried out in a sterile tissue culture hood. Shef-1 hESC were routinely cultured on Matrigel (BD) in TeSR1 media (Stem Cell Technologies). WA26 hESC (Wicell) were routinely cultured in Essential 8 Medium (Life Technologies) on human vitronectin (Life Technologies). Cultures were passaged twice per week using 0.5 mM EDTA solution (Sigma) to dissociate the colonies into smaller aggregates, which were then replated in medium containing 10 μM Y-27632 (Rho-associated kinase inhibitor) (Sigma). The culture medium was replaced daily.

Shef1 or WA26 hESC (Wicell) were incubated with 10 μM Y276352 (ROCK inhibitor) for 35 min at 37° C. Media was removed and the cells were washed with 5 ml PBS (—MgCl₂, —CaCl₂) (hereafter PBS (−/−)). 2 mL TrypLE Select® was added and cells incubated at 37° C./5% CO₂ in a humidified incubator for 6-8 min. DMEM KSRXF media was prepared as follows:

| | | Volume |
|---|---|---|
| Component | Catalogue Number | (mL) |
| Knockout (KO) DMEM | 10829-018 (Life Technologies) | 308 |
| Xeno-free Knockout Serum | 12618-012 (Life Technologies) | 80 |
| Replacement | | |
| Glutamax I | 35050 (Life Technologies) | 4 |
| 2-mercaptoethanol (70 uL | M3148 (Sigma) | 4 |
| diluted in 100 mL | | |
| KO DMEM) | | |
| Non-essential amino acids | 11140-035 (Life Technologies) | 4 |

TesR2 complete media (TesR2) was prepared as follows:

| Component | Catalogue Number | Volume (mL) |
|---|---|---|
| TesR2 basal media | 05860 (Stem cell technologies) | 78 |
| TesR2 5x supplement | 05860 (Stem cell technologies) | 20 |
| TesR2 250x | 05860 (Stem cell technologies) | 0.4 |
| supplement | | |

5 mL DMEM KSRXF media was added and pipetted up and down to achieve a single cell suspension. The suspension was transferred to a 15 mL falcon tube and centrifuged at 300×g for 4 min. The supernatant was aspirated and the pellet resuspended in 5 mL TesR2 complete Media®. The cell suspension was passed through a 40 μm cell strainer into a 50 mL falcon tube and the cell strainer was then washed with 1 mL TesR2 complete Media®. Cells were centrifuged at 1300 rpm for 4 min. The supernatant was aspirated and the pellet resuspended in 3 mL TesR2 complete Media® supplemented with 5 μM Y276352. T25 flasks were coated with the required matrix e.g. Matrigel or Fibronectin. Matrigel was thawed overnight in the fridge and diluted 1:15 with Knockout DMEM before use. Fibronectin was diluted 1:10 in PBS (−/−). 2.5 ml diluted matrix was used for coating a T25 flask and incubated for 3 hours at 37° C. Cells were counted and plated in the coated culture vessel at the appropriate density to obtain a monolayer. For a T25 flask, cells were seeded at a density of 240000 cells/cm² in a total volume of 10 ml in TeSR2 comprising 5 μM Y276352. This timepoint is designated as Day 0. 24 hours after plating (Day 1), media was aspirated and replaced with 10 mL/flask of TesR2 complete media (no Rock inhibitor). 48 hours after plating (Day 2), media was aspirated and replaced with 10 mL/flask DMEM KSRXF media containing 1 μM LDN193189 and 10 μM SB-431542. The media comprising the two inhibitors was replenished everyday. On Day 6, media was aspirated and replaced with 10 mL/flask DMEM KSRXF containing 100 ng/mL BMP4/7 heterodimer. Fresh media with BMP4/7 was replenished every day.

On Day 9, cells were replated as follows (Early Replate 1). First, culture vessels e.g T12.5 flasks, 96-well CellBind plates or 384-well CellBind plates were coated with the required matrix e.g. Matrigel, Fibronectin or Cellstart. Matrigel was thawed overnight in the fridge and diluted 1:15 with DMEM before use. Fibronectin was diluted 1:10 in PBS (−/−). Cellstart was diluted 1:50 in PBS (+MgCl₂, +CaCl₂) (hereafter PBS (+/+)). 1.5 ml diluted matrix was used for coating a T12.5 flask and incubated for 3 hours at 37° C. Next, 10 μM Y276352 was added to each T25 flask of cells (at Day 9 of the differentiation protocol) and incubated at 37° C. for 35 min. Media was aspirated and cells were washed twice with 5 mL PBS(−/−). 2.5 mL TrypLE Select® was added to each flask and the flask transferred to 37° C. for 15-25 min, until cells had lifted from the flask. 5 mL DMEM KSRXF media was added to each flask and used to wash the surface of the flask. The cell suspension was passed through a 40 μm cell strainer. Cells were centrifuged at 400×g for 5 min at room temperature. Supernatant was aspirated and the pellet resuspended in 10 mL DMEM KSRXF media (+5 μM Y276352). Supernatant was aspirated and the pellet resuspended in 10 mL DMEM KSRXF media (5 μM Y276352). Cells were counted and plated into coated culture vessels at a density of 500000 cells/cm². 24 h after replating (i.e at D10 which can also be noted D9-1 of the differentiation protocol), the media was changed to DMEM KSRXF+100 ng/mL activin A. Media was replenished with fresh activin A three times a week.

After D9-19 (i.e day D28), cells were replated to yield a homogeneous population of RPE cells (Early Replate 2). The media was aspirated and cells washed 2× with 5 mL PBS(−/−). 2.5 mL Accutase was added to each flask and incubated at 37° C. for about 35 min, until cells had lifted from the flask. 5 mL DMEM KSRXF media was added to each flask and used to wash the surface of the flask, before transferring the contents into a 50 mL falcon tube through a 70 μm strainer. Cells were centrifuged at 400×g for 5 min at room temperature. The supernatant was aspirated and the pellet resuspended in 10 mL DMEM KSRXF media. Cells were counted using a haemocytometer and plated in DMEM KSRXF media in coated culture vessels (e.g Cellstart 1:50 diluted in PBS (+/+)) at various densities e.g 120000/cm². Fresh media was replenished twice a week.

Cells were maintained in culture for 14 days. The resulting RPE cells were characterized inter alia by testing for expression of RPE markers (PMEL17, ZO1, BEST1, CRALBP) by immunocytochemistry and qPCR. More than 90% of the cells expressed the RPE marker PMEL17.

This protocol led to generation of RPE cells which express the RPE marker PMEL17 as well as other mature RPE markers such as CRALBP and MERTK.

This protocol involves treating a monolayer of pluripotent cells with SMAD inhibitors, preferably LDN193189 and SB-431542 followed by activation of the BMP pathway for example using a recombinant BMP4/7 heterodimer protein. Following LDN193189/SB-431542 and BMP4/7 treatment, cells are replated (Early Replate 1) and can be treated with activin A. Following treatment with activin A, cells can be replated for a second time (Early Replate 2) into basal media and maintained in culture to obtain pure RPE cells cultures. This leads to generation of homogeneous RPE cells cultures.

Without being bound to any theory, it is believed that the inhibition of the TGFβ signaling using the SMAD inhibitors leads to differentiation of hESC towards anterior neuroectoderm (ANE). Subsequent treatment with BMP pathway activators such as BMP4/7 induces differentiation of the ANE towards eye field. The subsequent replating and optional treatment with activin A led to a differentiation towards the RPE fate.

The present disclosure therefore provides a method for the robust and reproducible differentiation of hESCs to give rise to pure RPE cells. In addition, this protocol is easily scalable to give high yield. The above method can be used for reproducibly and efficiently differentiate hESCs into RPE cells in xeno-free conditions.

### Example 2

### Treatment with SMAD Inhibitors

This example illustrates the effect of SMAD inhibitors on hESCs.

2.1. Treatment with SMAD Inhibitors Leads to ANE Formation

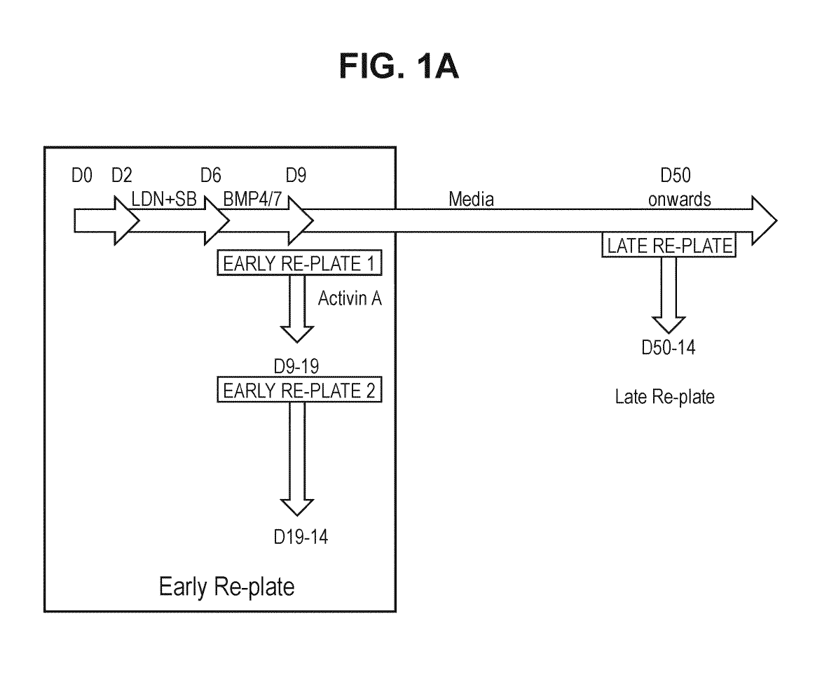

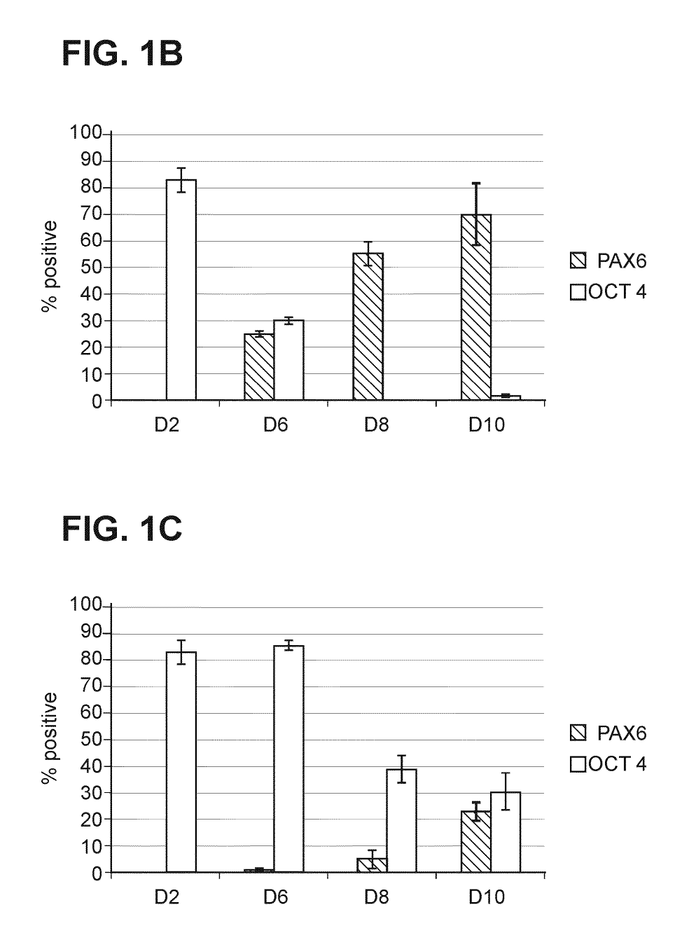

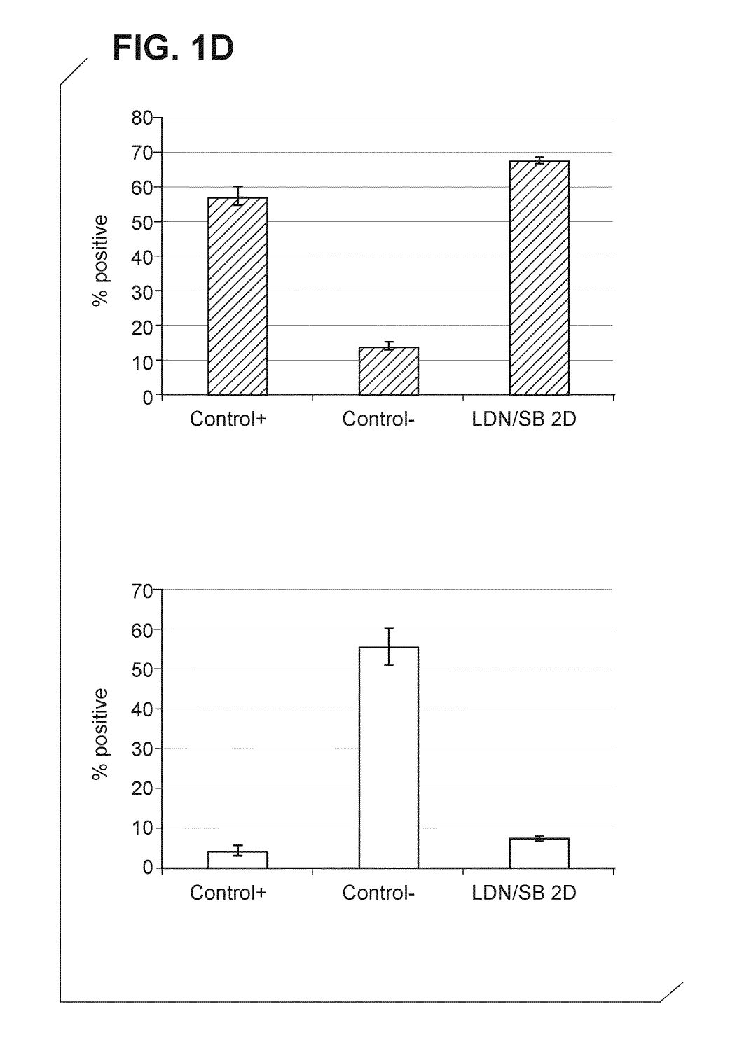

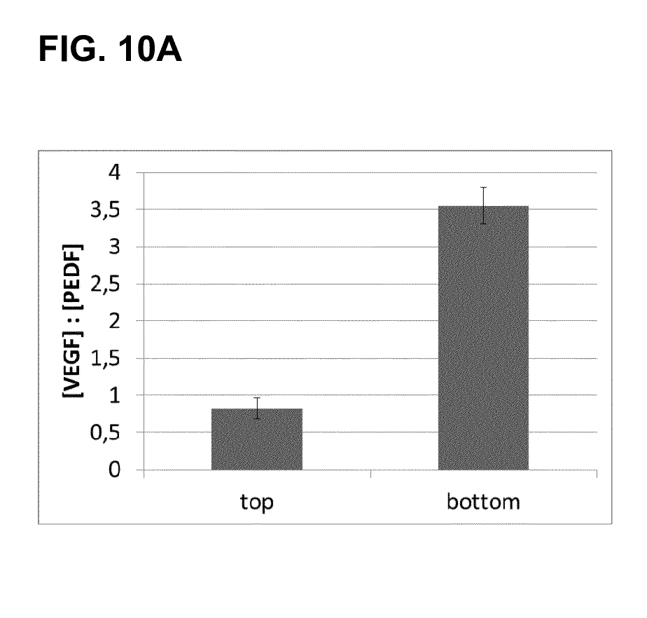

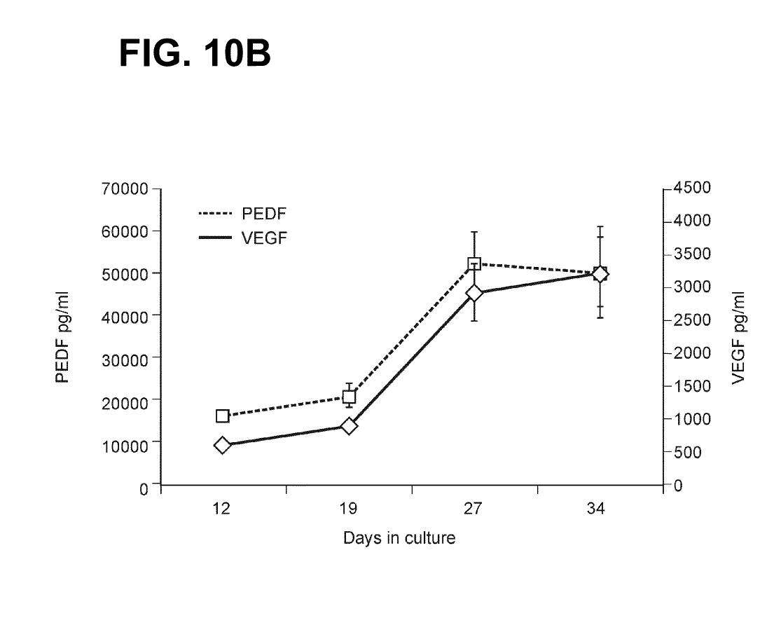

Shef-1 hESCs were seeded onto Matrigel coated 96-well plates at a density of 125000 cells/cm². On Day 2 post seeding, cells were treated with 1 μM LDN193189 and 10 μM SB-431542 and samples were fixed at Day 2, Day 6, Day 8 and Day 10. Immunocytochemistry was carried out for PAX6 (marker of ANE) expression and OCT4 (marker of pluripotent hESCs) downregulation. A uniform induction of PAX6 protein and a uniform decrease of OCT4 over the time course of differentiation was seen in samples induced with LDN193189 and SB-431542 (FIG. 1B). This was observed not only on the whole surface of one well of a 96-well plate but similarly in all the wells within the plate indicating a robust induction with low inter/intra plate variability. In contrast, samples not treated with LDN193189 and SB-431542 and maintained in media alone expressed low levels of PAX6 and higher levels of OCT4 at the end of the timecourse indicating that efficient induction of ANE did not occur in the absence of LDN193189 and SB-431542 (FIG. 10).

2.2. Treatment with SMAD Inhibitors for Two Days

Shef-1 hESCs were seeded onto Matrigel coated 96-well plates at a density of 125000 cells/cm². On Day 2 post seeding, cells were treated with 1 μM LDN193189 and 10 μM SB-431542 for different lengths of time as described in Table 1.

TABLE 1

| | Day | Day | Day | Day | Day | Day |
|---|---|---|---|---|---|---|
| | 2-0 | 2-1 | 2-2 | 2-3 | 2-4 | 2-5 |
| Control+ | LDN/SB | LDN/SB | LDN/SB | LDN/SB | LDN/SB | LDN/SB |
| Control− | DMEM | DMEM | DMEM | DMEM | DMEM | DMEM |
| | KSRXF | KSRXF | KSRXF | KSRXF | KSRXF | KSRXF |
| LDN/SB | LDN/SB | LDN/SB | DMEM | DMEM | DMEM | DMEM |
| 2 day | | | KSRXF | KSRXF | KSRXF | KSRXF |

Cells were immunostained for PAX6 and OCT4. The level of PAX6 upregulation and OCT4 downregulation was similar for all conditions tested (FIG. 1C). This shows that at least 2 days of LDN193189/SB-431542 results in ANE induction.

### Example 3

### Induction of RPE Markers

### Example 3.1

### Induction of MITF by Activation of BMP Pathway

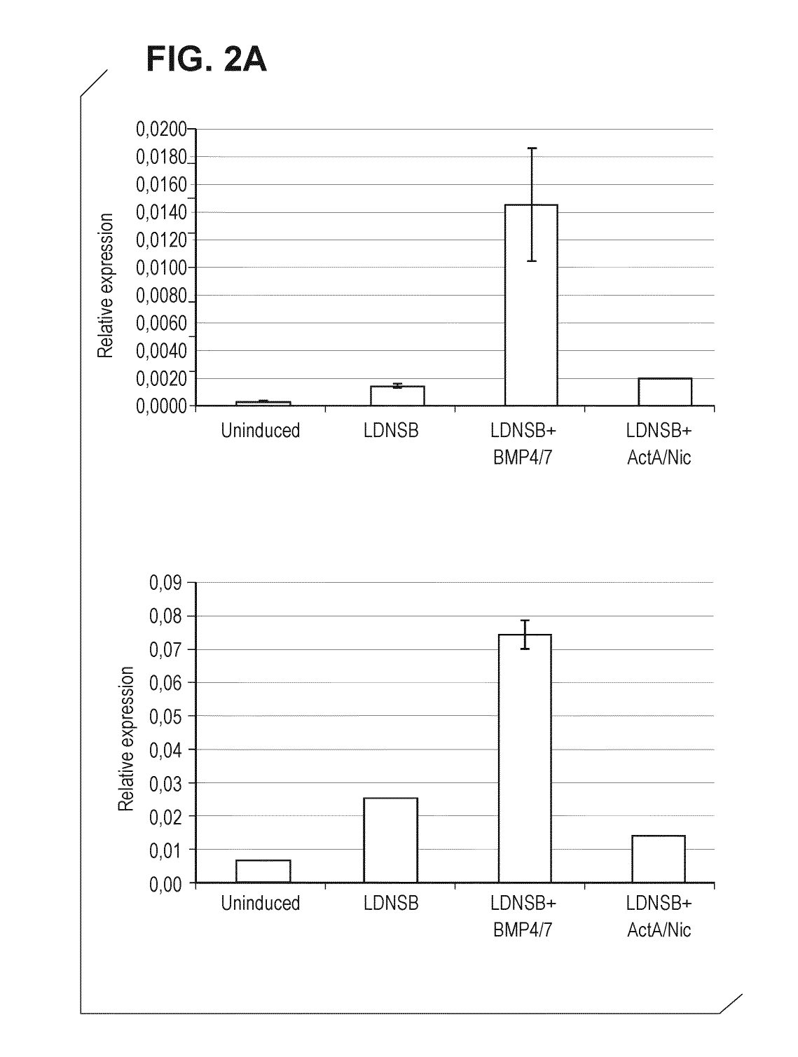

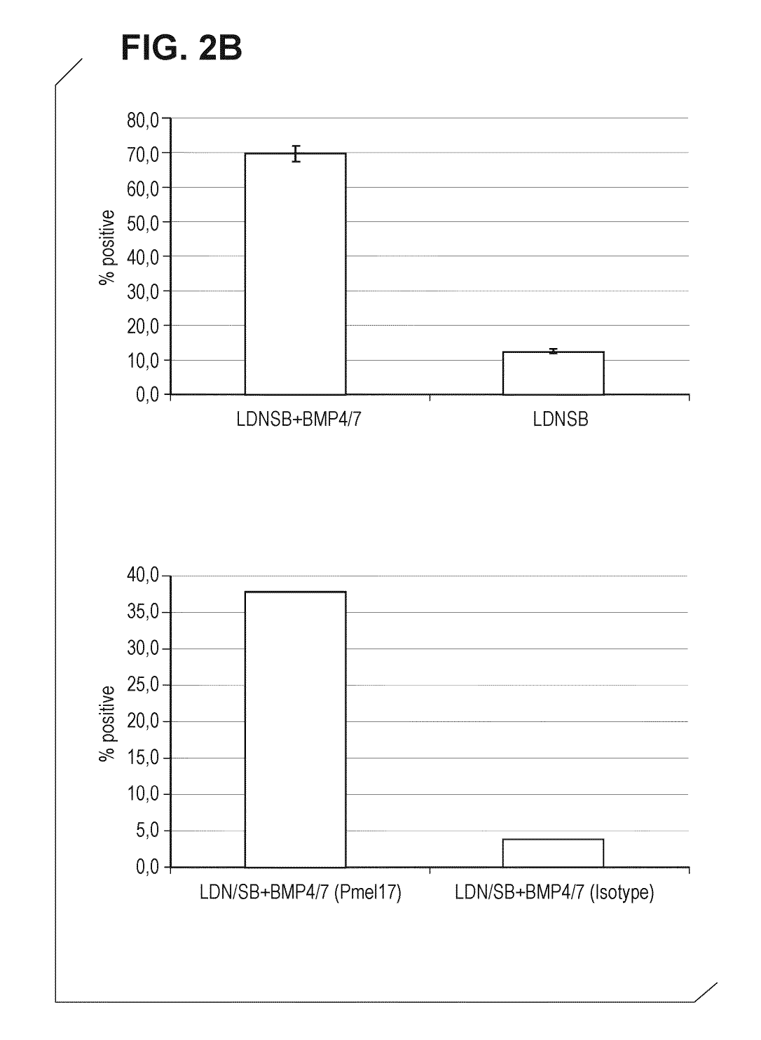

This example illustrates the effect of a BMP pathway activator on RPE marker expression. Shef-1 hESCs were seeded onto Matrigel coated 96-well plates at a density of 125000 cells/cm². On Day 2 post seeding 1 μM LDN193189 and 10 μM SB-431542 were applied for 4 days. Cells for the uninduced control were left untreated. On Day 6, 100 ng/ml BMP4/7 or 100 ng/ml activin A+10 mM Nicotinamide or nothing was added to the media for 3 days. On Day 9, BMP4/7 or activin A and Nicotinamide were withdrawn and cells were treated with DMEM KSRXF alone for 4 days. Samples were prepared for RNA extraction and qPCR analysis. The results are summarized in FIG. 2A.

BMP4/7 induced expression of RPE genes e.g MITF and PMEL17 as compared to uninduced or LDN193189/SB-431542 only treated controls. Furthermore, activin A+Nicotinamide could not substitute for BMP4/7 (FIG. 2A). Immunocytochemistry was also performed on samples that were treated with LDN193189/SB-431542 followed by BMP4/7 which confirmed expression of RPE markers e.g MITF and PMEL17 (FIG. 2B). These results demonstrate that a BMP pathway activator strongly induces MITF expression and PMEL17 expression.

### Example 3.2

Shef-1 hESCs were treated with 1 μM LDN193189 and 10 μM SB-431542 from Day 2 to Day6 followed by 100 ng/ml BMP4/7 from Day6 to Day9 (induced cells). Uninduced cells are maintained without exposure to both LDN/SB and BMP4/7. Immunocytochemistry was performed for PAX6, LHX2, OTX2, SOX11 and SOX2 which are markers known to be expressed when cells are committed to the eye field fate. OCT4, a marker of pluripotency, is downregulated from Day 2 to Day9 in induced cells. PAX6, LHX2, OTX2, SOX11 and SOX2 are upregulated from Day2 to Day9 and this upregulation is not achieved in uninduced samples. This shows that the directed differentiation protocol induces cells towards an eye field state which is then committed towards an RPE fate.

### Example 4

### Use of Alternative BMP Pathway Activators

This example illustrates the effect of various BMP pathway activators on RPE marker expression.

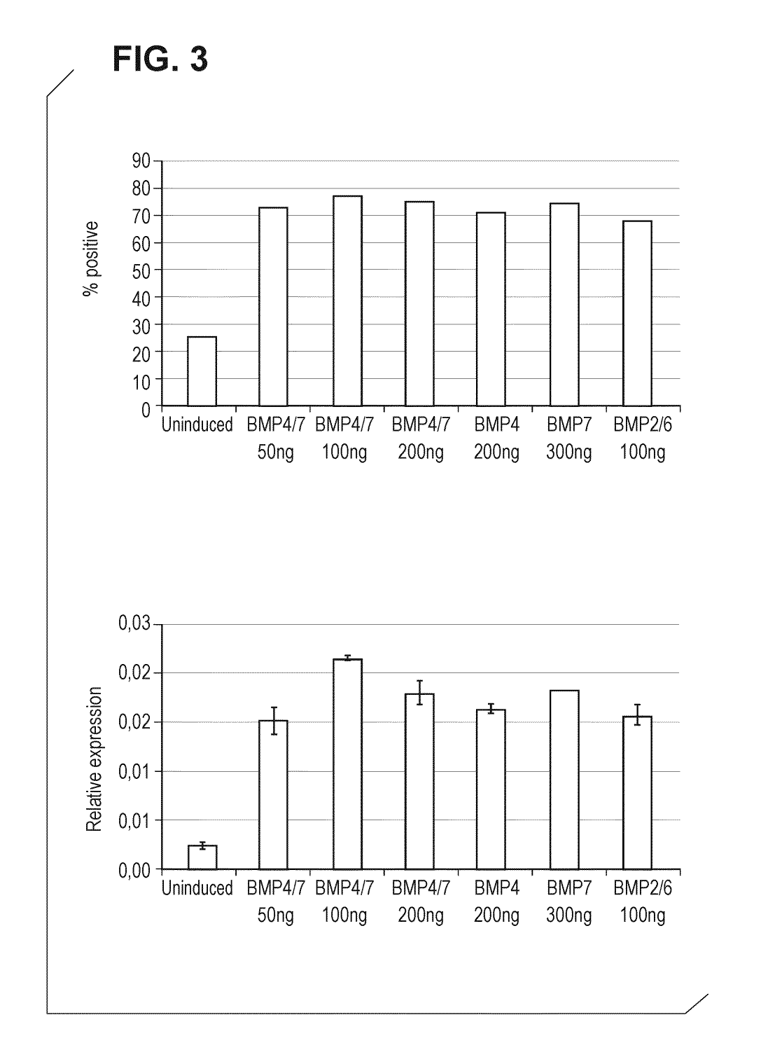

Shef-1 hESCs were seeded onto Matrigel coated 96-well plates at a density of 125000 cells/cm². On Day 2 post seeding, 1 μM LDN193189 and 10 μM SB-431542 were applied for 4 days. On Day 6, 50-200 ng/ml BMP4/7 heterodimer or 200 ng/ml BMP4, 300 ng/ml BMP7, 100 ng/ml BMP2/6 were added for a period of 3 days. On Day 9, BMPs were withdrawn and cells maintained in DMEM KSRXF alone for 4 days. On Day 13, MITF expression was tested by Immunostaining and qPCR analysis. Treatment with either BMP4/7 heterodimer or other BMPs induced expression of MITF to a similar level (FIG. 3). This showed that BMP4/7 could be substituted with other BMPs.

These results demonstrate that different BMP pathway activators can be used to induce MITF expression.

### Example 5

### First Replating Step

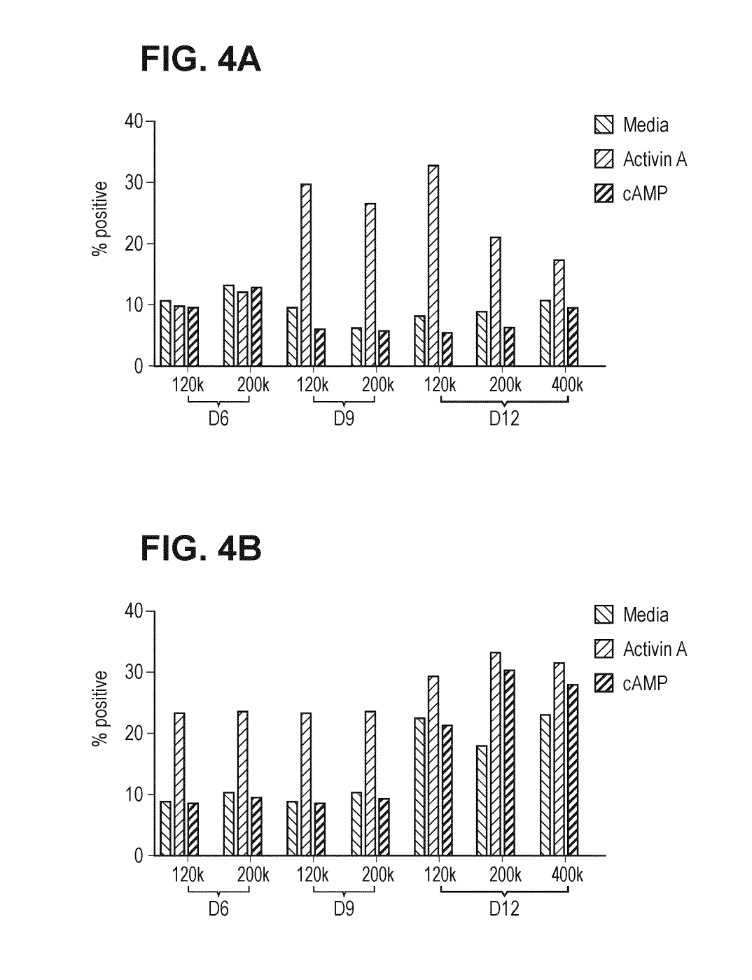

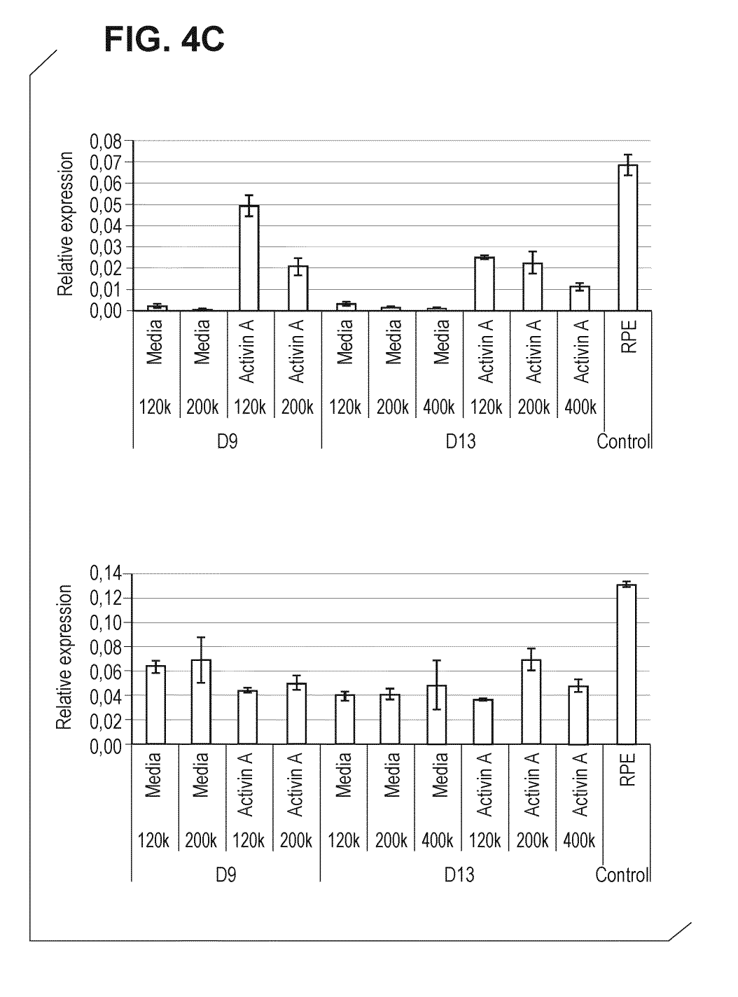

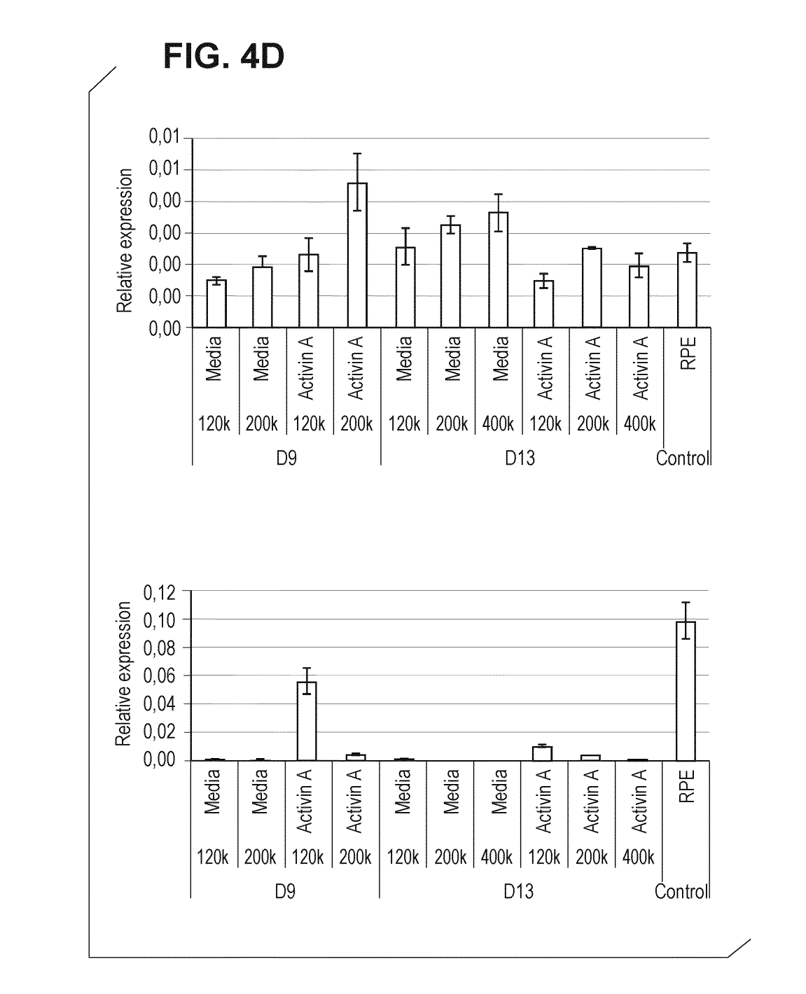

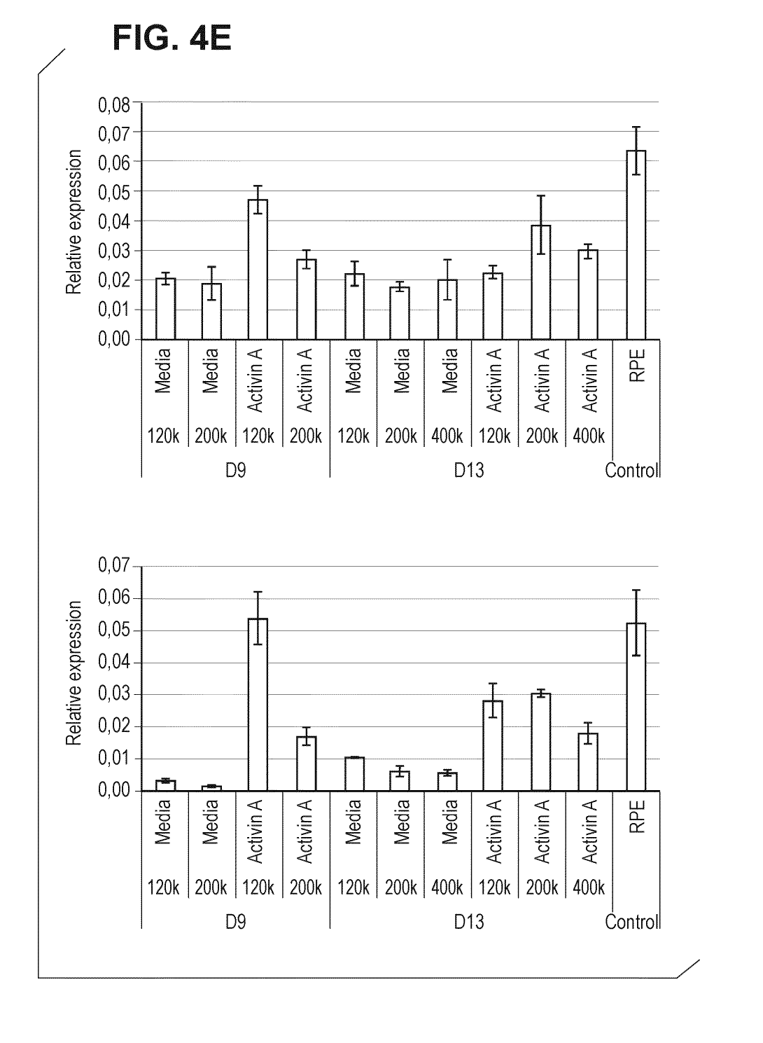

Shef-1 hESCs were seeded onto a Matrigel coated T25 flask at a density of 240000 cells/cm². On Day 2 post seeding, 1 μM LDN193189 and 10 μM SB-431542 were applied for 4 days. On Day 6, 100 ng/ml BMP4/7 was added to the media for 3 days. Cells were replated at either Day 6, Day 9 or Day 12 of the differentiation protocol into DMEM KSRXF alone or DMEM KSRXF supplemented with either 100 ng/ml activin A, 0.5 mM cAMP or 100 ng/ml BMP4/7 at various densities. Cells replated at Day 6 were maintained for 3 days post replating in DMEM KSRXF supplemented with 100 ng/ml BMP4/7 before switching to activin A, cAMP or BMP4/7. Cells replated at Day 12 were maintained from Day 9 to Day 12 in DMEM KSRXF alone before replating. Replated cells did not survive in the presence of BMP4/7 and this condition was discarded from subsequent analysis. Mature RPE cells sample obtained by spontaneous differentiation as disclosed in Example 10 (a) was used as a control to compare the similarity between the populations obtained upon the first replating step of directed differentiation and mature RPE cells. 19 days post replating, cells were fixed for immunocytochemistry and samples were collected for qPCR. Immunocytochemistry with mature RPE markers e.g CRALBP and MERTK showed that replating at D9 in the presence of activin A was optimum and yielded high levels of RPE marker expression (FIGS. 4A and 4B). QPCR analysis with a panel of markers also indicated Day 9 to be the optimum time for replating (FIGS. 4C, 4D and 4E). Similar results were obtained when cells were cultured on different matrices e.g Matrigel, Cellstart or Fibronectin before and after replate.

### Example 6

### Duration of Exposure to Activin A

This example illustrates the effects of activin A exposure duration on RPE differentiation.

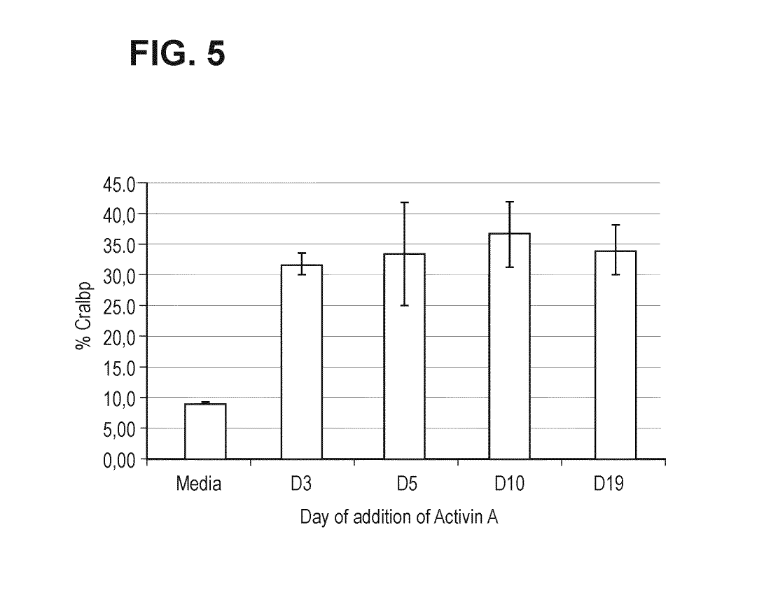

WA26 hESCs (Wicell) were seeded onto Matrigel coated T25 flask at a density of 240000 cells/cm². On Day 2 post seeding, 1 μM LDN193189 and 10 μM SB-431542 were applied for 4 days. On Day 6, 100 ng/ml BMP4/7 was added to the media for 3 days. On Day 9, cells were replated into 96 well CellBind plates coated with Matrigel or Cellstart at a density of 500000 cells/cm². The cells were maintained in either DMEM KSRXF alone or DMEM KSRXF supplemented with 100 ng/ml activin A for different lengths of time e.g 3 days, 5 days, 10 days or 18 days. At D9-18, cells were fixed for immunostaining and stained for CRALBP, a marker of RPE cells. The level of CRALBP expression was similar for all activin A treatments tested (FIG. 5). These results demonstrate that a short exposure to activin A is sufficient for inducing RPE cells differentiation.

### Example 7

### Second Replating Step at Various Densities

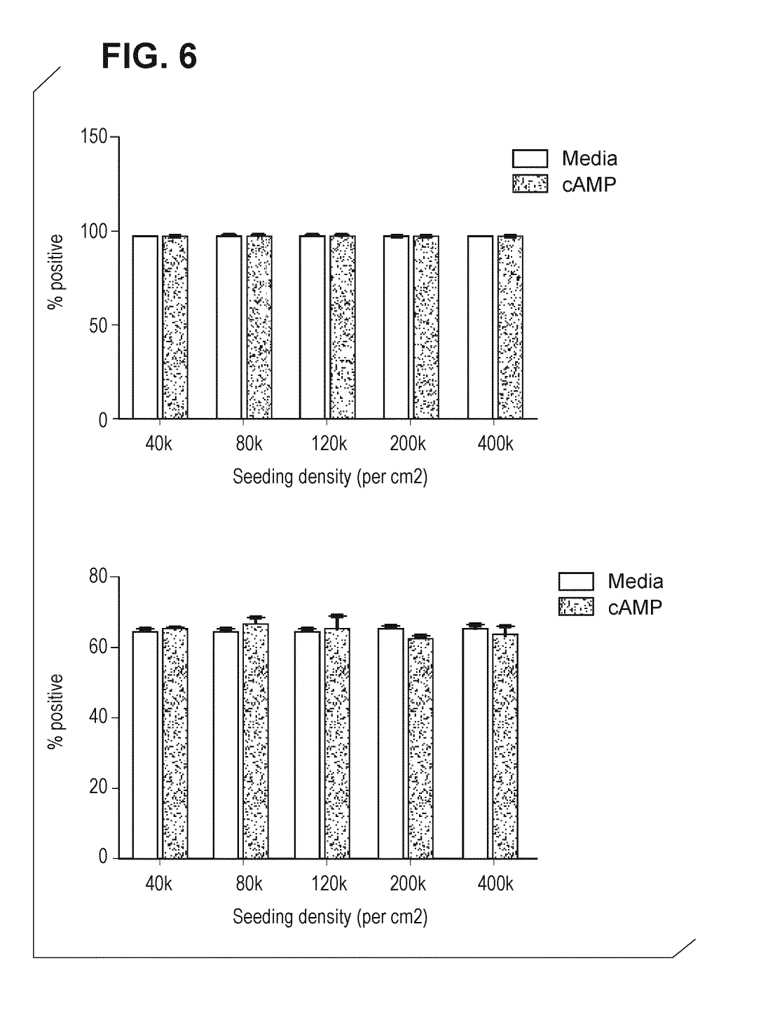

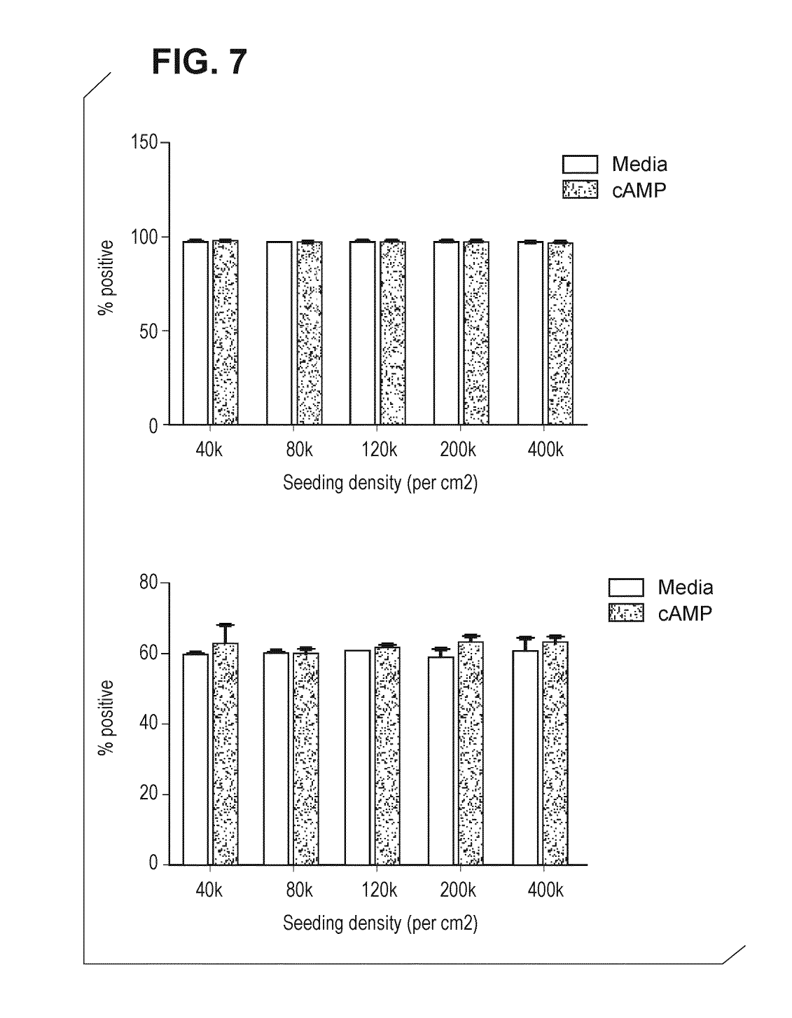

WA26 hESCs (Wicell) were seeded onto Matrigel coated T25 flask at a density of 240000 cells/cm². On Day 2 post seeding, 1 μM LDN193189 and 10 μM SB-431542 were applied for 4 days. On Day 6, 100 ng/ml BMP4/7 was added to the media for 3 days. On Day 9, cells were replated into T12.5 flasks coated with either Matrigel or Cellstart at a density of 500000 cells/cm². The cells were maintained in DMEM KSRXF supplemented with 100 ng/ml activin A for 19 days. At D9-19, cells were replated into Cellstart coated 96-well or 384-well plates at various densities (Early Replate 2). The cells were maintained for 20 days in media alone or media supplemented with 0.5 mM cAMP. At D9-19-20, cells were fixed for immunostaining for RPE markers. Both 96 and 384 well formats yielded similar results of >95% expression of PMEL17 and about 60% expression of CRALBP (FIGS. 6 and 7). Furthermore, expression of ZO1, another marker of mature RPE cells was confirmed by immunostaining.

### Example 8

### Directed Differentiation with Late Replating

The protocol up to Day 9 was identical to the protocol disclosed above in Example 1.

On Day 9, media was replaced with 10 ml DMEM KSRXF per flask. The cells were maintained in this media until Day 50 with fresh media change thrice a week. Around Day 50, cobble-stoned cells were visible in the flask interspersed with other cells of different morphologies. Also, the central area of the flask had a distinct morphology with several areas of high density that had neuronal projections.

To carry out replating, media was removed from the flask and cells washed once with 5 mL PBS (−/−). 5 ml PBS was added to the flask and the central dense area was scraped using a cell scraper and discarded. The flask was washed again with 5 ml PBS (−/−). 5 mL Accutase was added to the flask and incubated at 37° C. for about 50 min, until cells had lifted from the flask. 5 mL DMEM KSRXF media was added to each flask and used to wash the surface of the flask, before transferring the contents into a 50 mL falcon tube through a 70 μm strainer. Cells were centrifuged at 400×g for 5 min at room temperature. The supernatant was aspirated and the pellet resuspended in 10 mL DMEM KSRXF media. Cells were counted using a haemocytometer and plated in DMEM KSRXF media in coated culture vessels (e.g Cellstart 1:50 diluted in PBS (+/+) at various densities e.g 200000/cm². Fresh media was replenished twice a week.

Cells were maintained in culture for 14 days. The resulting RPE cells were characterized by testing for expression of RPE markers (PMEL17, ZO1, BEST1, CRALBP) by immunocytochemistry and qPCR. The functionality of RPE cells was tested by analysing secretion of VEGF and PEDF proteins which is an indicator of RPE cells maturity.

The present disclosure therefore provides a method for the robust and reproducible differentiation of hESCs to give rise to RPE cells. In addition this protocol is easily scalable to give high yield. The above method can be used for reproducibly and efficiently differentiate hESCs into RPE cells in xeno-free free conditions

### Example 9

### Late Replating on Different Coatings

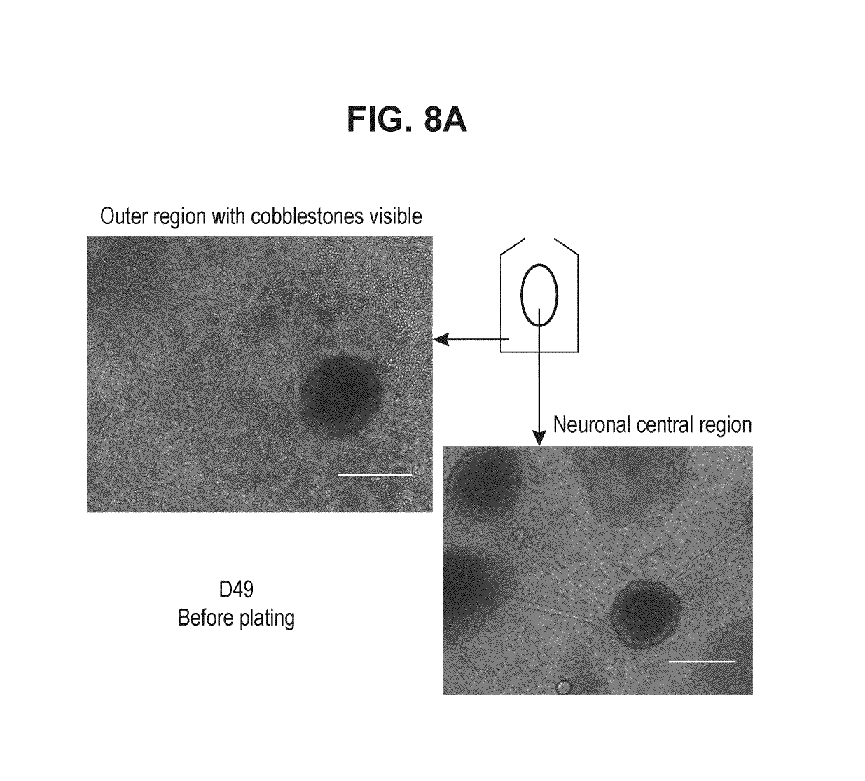

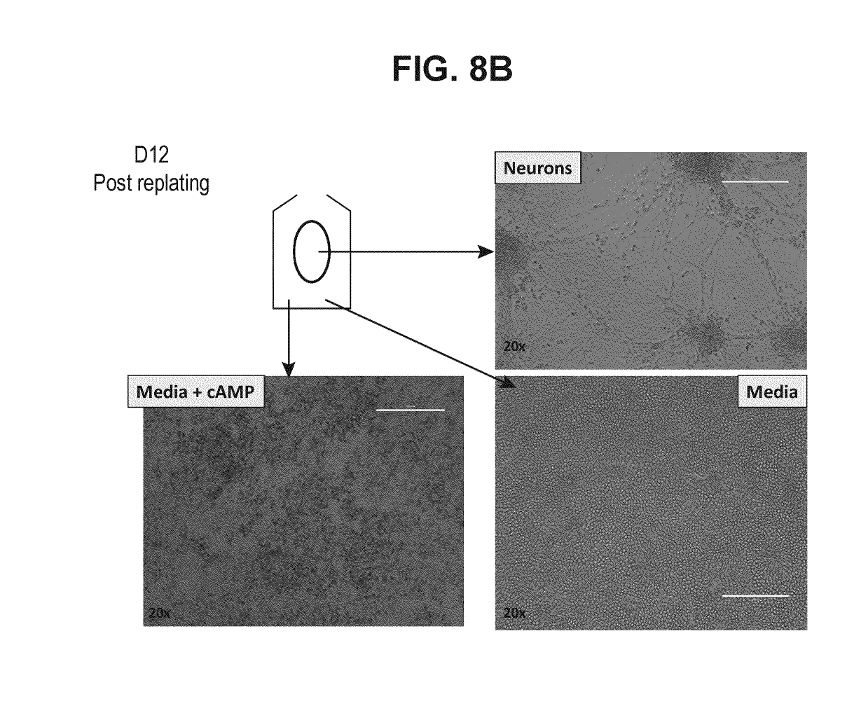

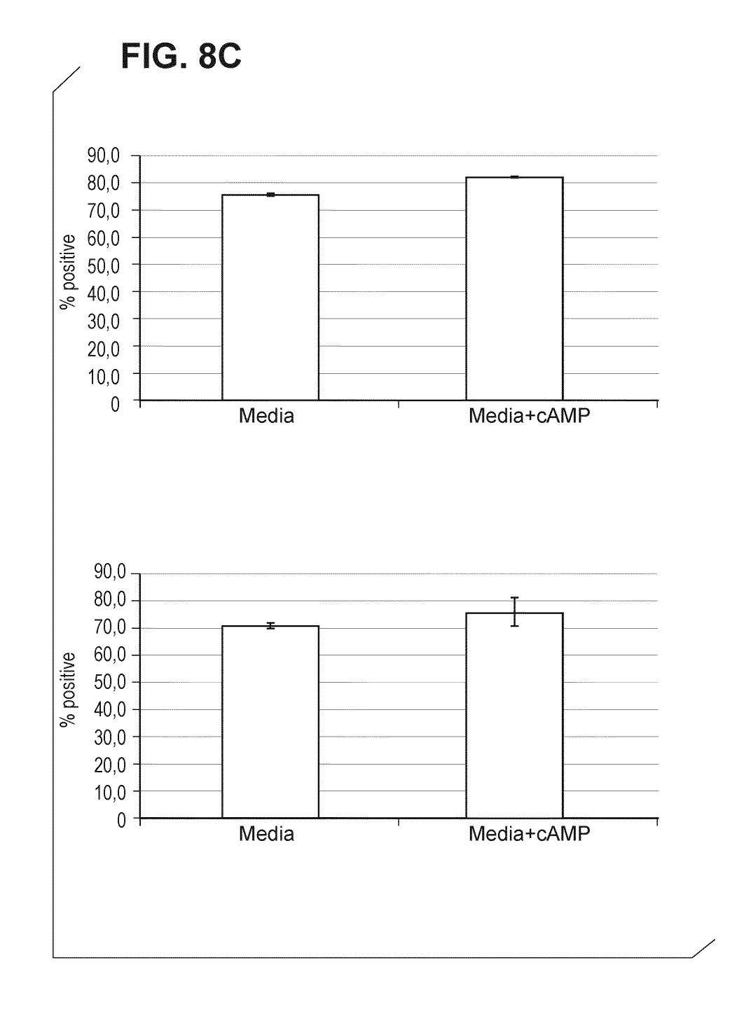

Shef-1 hESCs were seeded onto Matrigel coated T25 flask at a density of 240000 cells/cm². On Day 2 post seeding, 1 μM LDN193189 and 10 μM SB-431542 were applied for 4 days. On Day 6, BMP4/7 was added to the media for 3 days. Cells were then maintained in media alone until Day 50. On Day 50, the outer edge of the flask, where cobblestoned cells were visible (FIG. 8A), was collected and seeded onto Matrigel, Cellstart or Fibronectin coated plates in 96-well or 48-well format at a density of 200000 cells/cm². The inner dense area of the flask, where cobblestones were not visible, was collected and seeded separately (FIG. 8A). Replated cells were maintained in media alone or media supplemented with 0.5 mM cAMP. Cells replated from the inner dense area gave rise to a high proportion of neurons and were discarded. Cells cultured from the outer edge gave rise to cobblestoned cells which were more pigmented in the presence of cAMP (FIG. 8B). Furthermore, cells expressed RPE markers such as PMEL17, ZO-1, CRALBP, Bestrophin and MERTK as observed by immunostaining. Quantification for PMEL17 and CRALBP immunostaining 15 days post replating showed greater than 70% expression of both markers (FIG. 8C). Similar phenotypes were obtained on all coatings tested.

### Example 10

### RPE Cells Obtained by Directed Differentiation Closely Resemble Spontaneously Differentiated RPE Cells

a) Preparation of Spontaneously Differentiated RPE Cells

Shef-1 hESCs were cultured as colonies either on inactivated mouse embryonic fibroblasts (iMEF) or inactivated human dermal fibroblasts (iHDFs) in Knockout DMEM (GIBCO) supplemented with 20% KSR (GIBCO), 1% non-essential amino acid solution (GIBCO), 1 mM L-glutamine, 0.1 mM β-mercaptoethanol, 30 μg/ml gentamicin (GIBCO) and 4 ng/ml human recombinant bFGF, or feeder free on Matrigel (BD) in mTesR1 medium (StemCell Technologies). All cultures were fed daily until superconfluent (approximately 2 weeks post seeding) before changing to Knockout DMEM media as above but without bFGF. Flasks were fed thrice weekly until RPE colonies had appeared and were large enough to cut out. The colonies were then excised with a scalpel, washed with PBS (−/−) and incubated with Accutase (GIBCO) for 1-1.5 hrs in a shaking water bath. Dissociated RPE cells were strained through a 70 μm cell strainer, centrifuged at 700×g for 5 min and resuspended in warm Knockout DMEM media without bFGF as above. RPE cells were counted and seeded (typically at 38000-50000 cells/cm²) into 48 well plates coated with extracellular matrix (typically 1:50 CellStart (Life Technologies) in PBS (+/+) coated for 2 hrs in the cell culture incubator). These were typically cultured for 7 or 16 weeks (cells seeded on day 0), feeding twice weekly with 0.5 ml/well, before performing RNA extraction.

De-differentiated RPE cells samples were produced by the same protocol as above but cells were seeded at 2500 cells/cm² for de-differentiation and were cultured for 4 or 5 weeks.

b) Comparison of Samples from RPE Cells Obtained by Directed Differentiation and Spontaneous Differentiation

Samples obtained from directed differentiation as disclosed in Example 8 were compared with samples obtained by spontaneous differentiation for a panel of RPE cells and other markers by quantitative PCR. The spontaneously differentiated RPE cells had been in culture for either 7 or 16 weeks. De-differentiated samples were used as a control as these cells did not achieve an epithelial phenotype and instead remained fusiform and de-differentiated. These were included to see whether the genes tested by qPCR were capable of differentiating between epithelial RPE cells and non-RPE like cells.

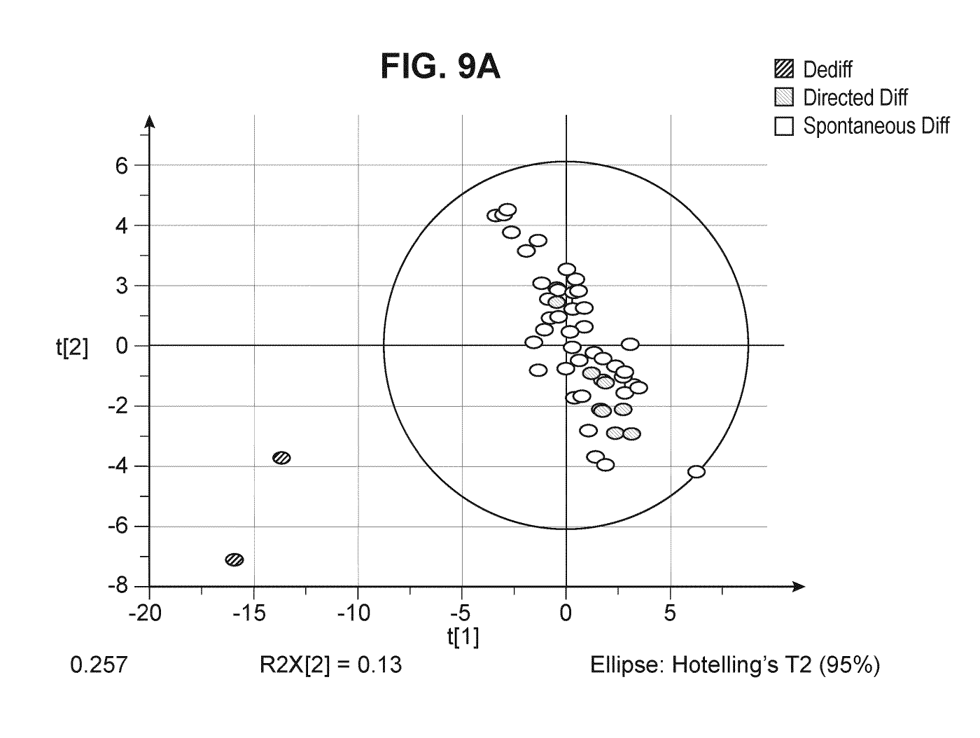

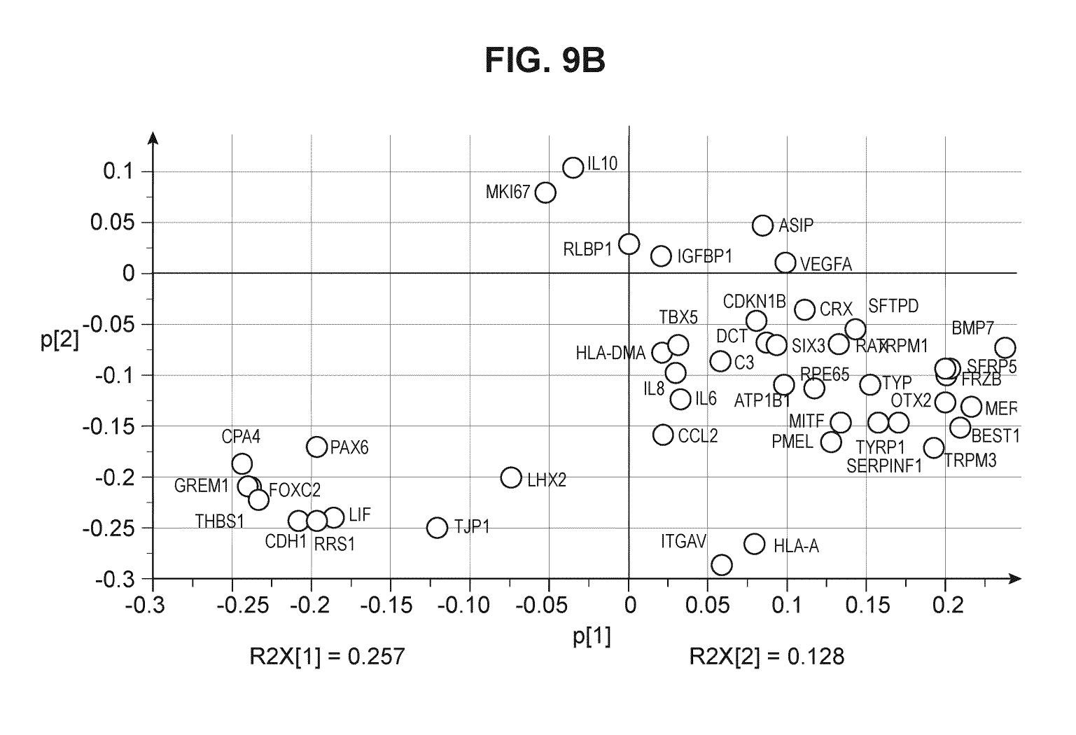

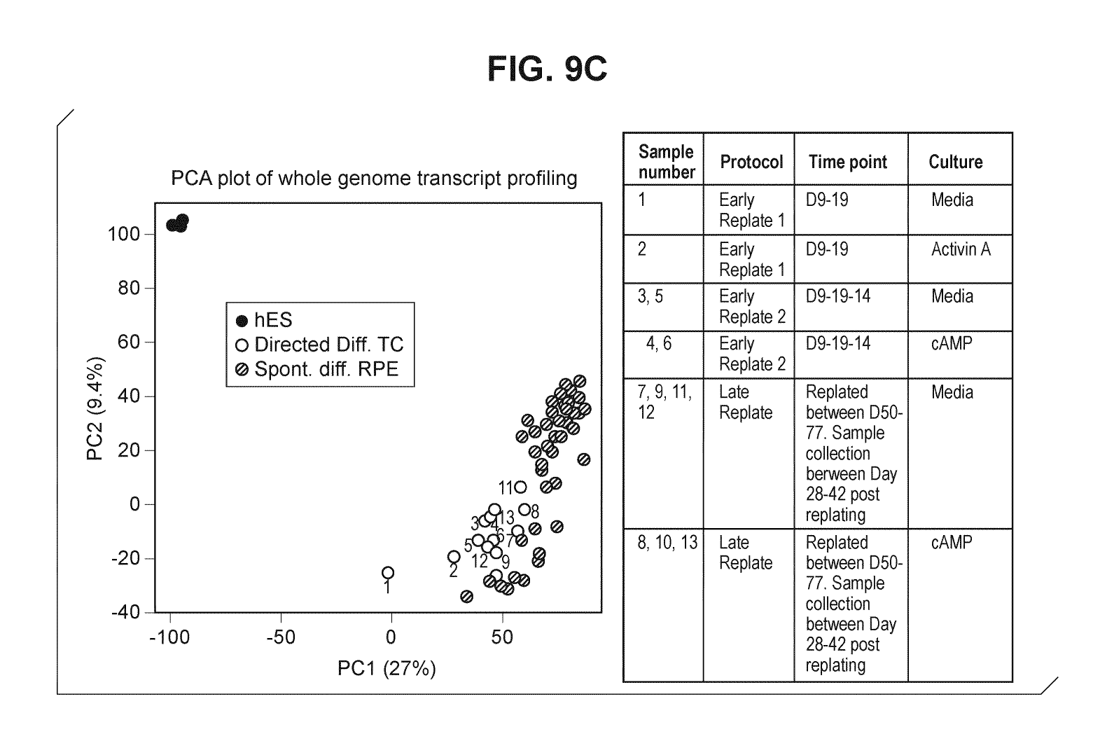

FIG. 9A shows a Principal Component Analysis (PCA) plot of 7 RPE cells samples generated by directed differentiation along with RPE cells generated by spontaneous differentiation as well as de-differentiated controls. Loadings plots of the PCA model of the mean-centred, unit variance scaled mRNA transcript data are also shown which shows the contribution of each of the genes tested to the clustering of the samples (FIG. 9B). PCA was used to visualise the overall variation of the samples. The scores plot of the first 2 components revealed that the de-differentiated samples clustered outside the Hotelling's T2 ellipse and were characterised by lower levels of the markers positively correlated with the RPE phenotype: MERTK, PMEL17, Tyrosinase, Bestrophin, RPE65 and CRALBP indicating that they did not resemble differentiated RPE cells and that the genes tested were capable of distinguishing between the RPE and non-RPE phenotype. Furthermore, RPE cells generated by directed differentiation clustered with the RPE cells samples generated by spontaneous differentiation and so possess the appropriate characteristics associated with differentiated RPE cells.

Next, whole genome transcript profiling of RPE cells obtained by Directed Differentiation (both Early and Late replating as disclosed in Examples 1 and 8) was performed and compared with the transcript profile of RPE cells obtained by Spontaneous Differentiation. The clustering of samples evident from the principal component analysis shown in FIG. 9C demonstrates that cells derived from both early and late replating protocols as disclosed in Examples 1 and 8 have a genome-wide gene expression profile similar to RPE cells derived from spontaneous differentiation, but distinct from hESCs.

In a related study, it was confirmed that RPE cells obtained by Spontaneous Differentiation were similar to native RPE cells in terms of their gene expression signature.

### Example 11

### RPE Cells Obtained by Directed Differentiation Secrete VEGF and PEDF Proteins

a) RPE Obtained by the Early Replating Method

Cells obtained after Replate 2 (D9-19-50) of the early replating protocol disclosed in example 1 were seeded onto Transwells® at a density of 116000 cells/Transwell® and cultured for a period of 10 weeks. The two chambers of the Transwell® were maintained as separate and media were not allowed to mix. Media were collected from the bottom and top chamber and analysed for secretion of VEGF and PEDF. As shown in FIG. 10A, the ratio of [VEGF]:[PEDF] is higher in the media collected from the bottom chamber and lower in the media from the top chamber indicating higher basolateral secretion of VEGF and higher apical secretion of PEDF. This indicates that the RPE obtained by directed differentiation method disclosed herein are polarized and functional.

b) RPE Obtained by the Late Replating Method

For late replating, Shef-1 hESCs were seeded onto Matrigel coated T25 flask at a density of 240000 cells/cm². On Day 2 post seeding, 1 μM LDN193189 and 10 μM SB-431542 were applied for 4 days. On Day 6, 100 ng/ml BMP4/7 was added to the media for 3 days. From Day 9 onwards, cells were then maintained in media alone until Day 64 when outer edges of the flask were collected and replated onto Matrigel coated Transwells® at a density of 400000 cells/cm². The Transwell® were fed by overflowing twice a week. Spent media was collected from Day 12 post seeding on Transwell® onwards at regular intervals for quantification of VEGF and PEDF levels. VEGF and PEDF measurements were made using the ‘Meso Scale Discover’ (MSD)-based multianalyte approach, according to the manufacturer protocols. As shown on FIG. 10B, VEGF and PEDF levels increase with time in culture indicating active secretion by RPE cells, which is an indicator of maturity. These results demonstrate that the cells obtained by the method described herein are RPEs.

### Example 12

### Expansion of RPE Cells

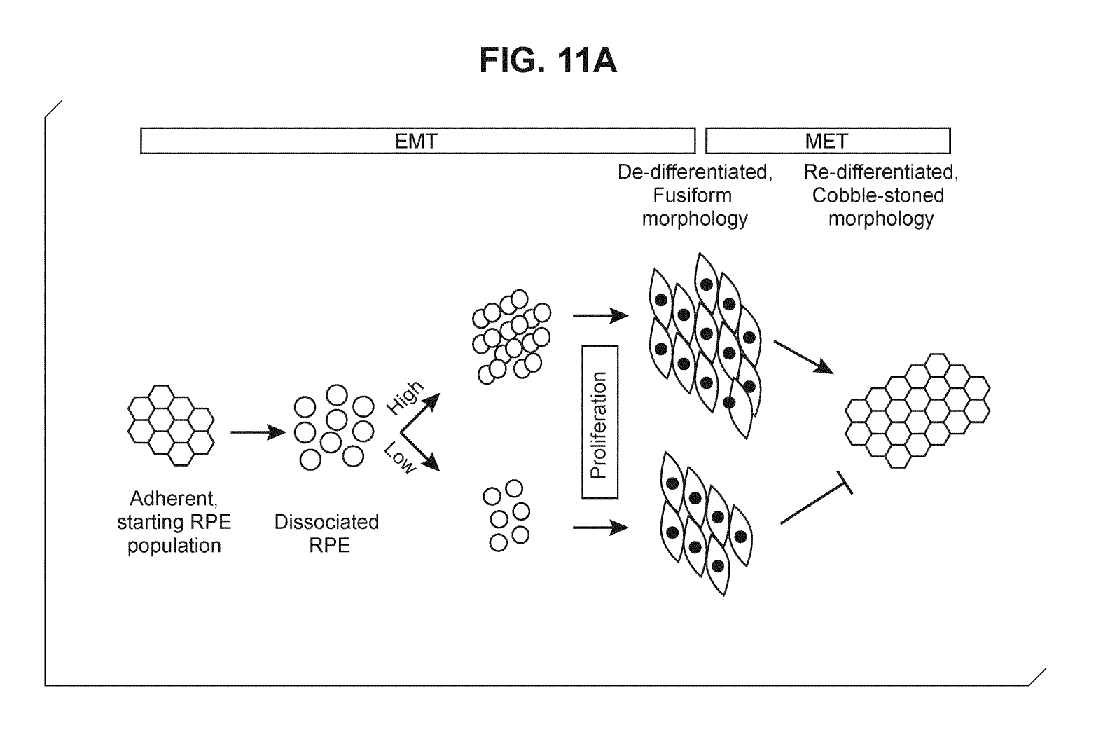

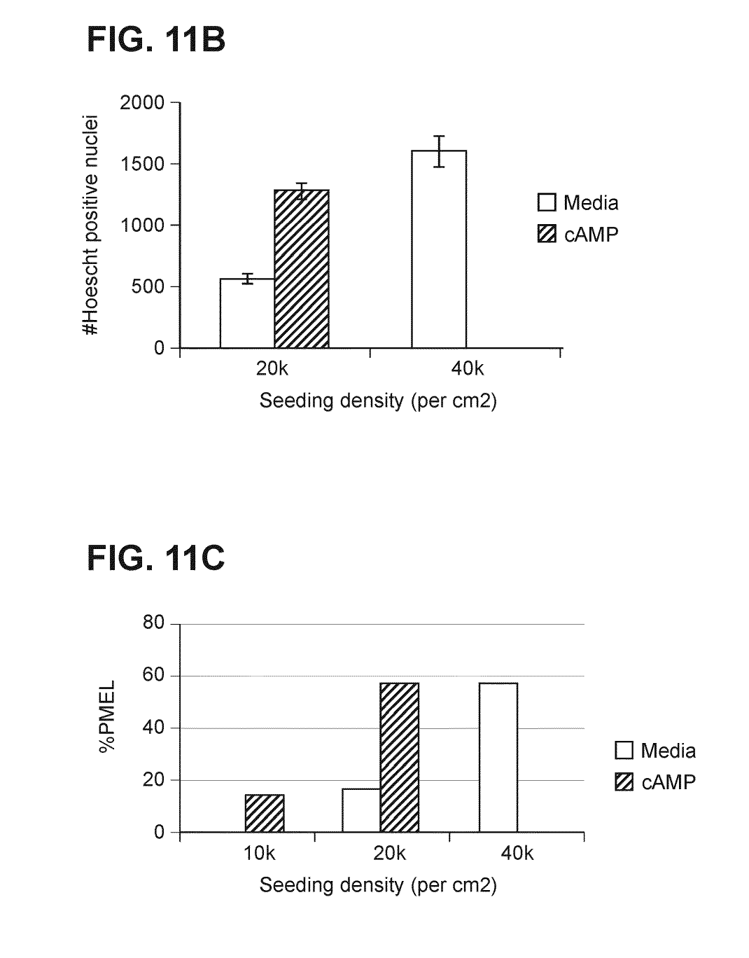

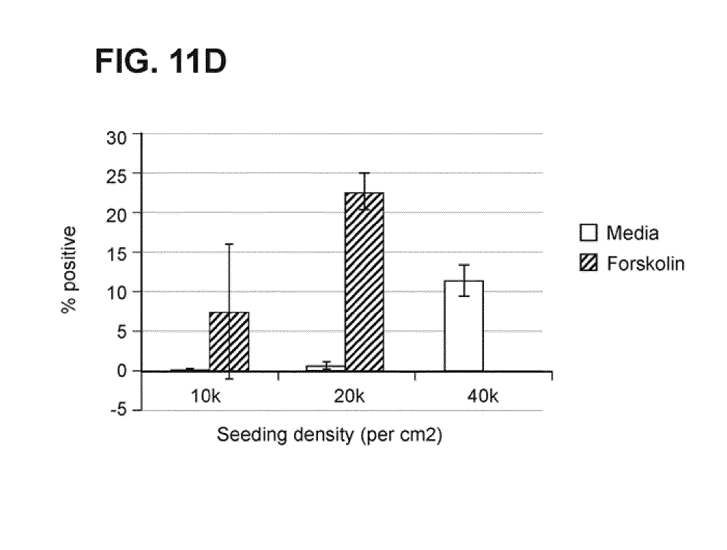

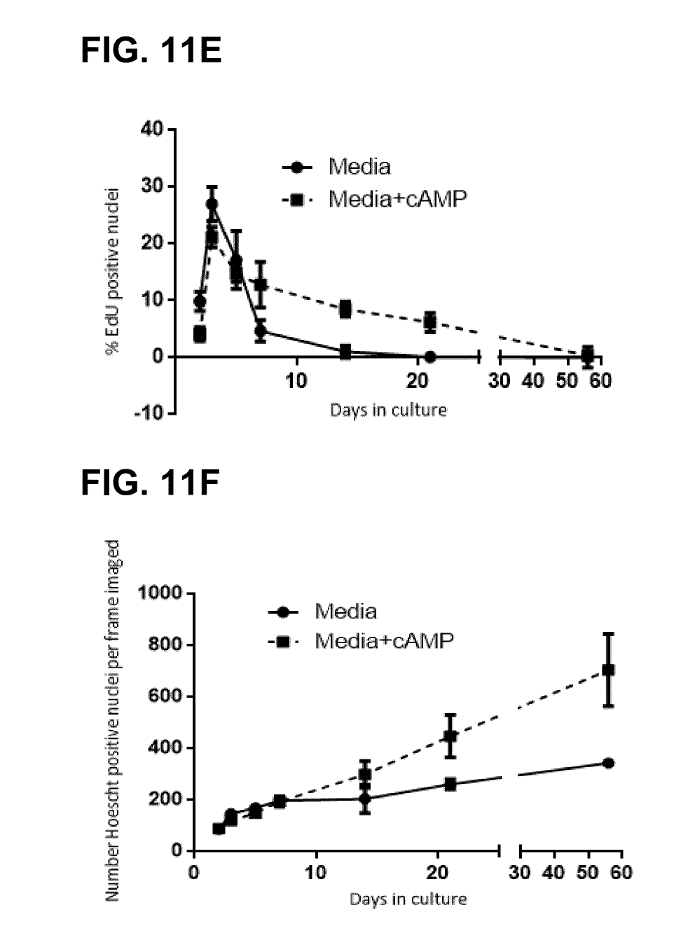

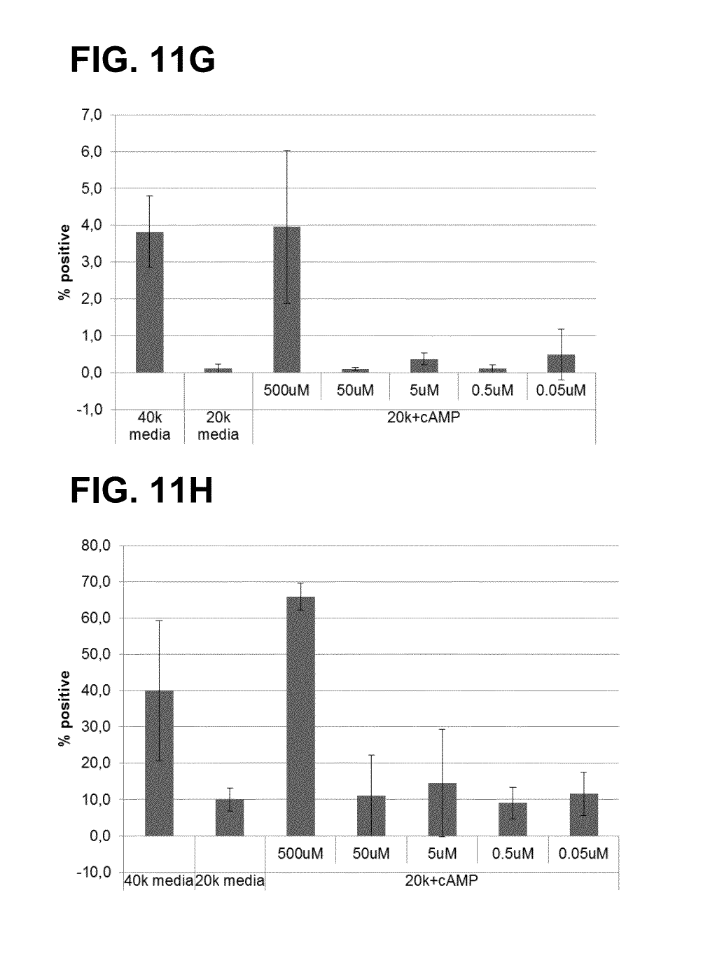

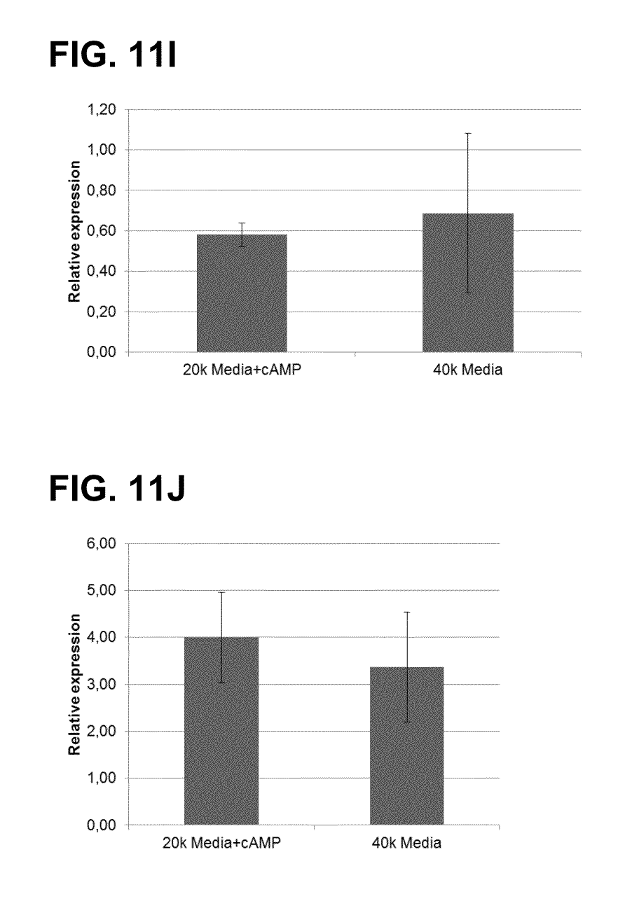

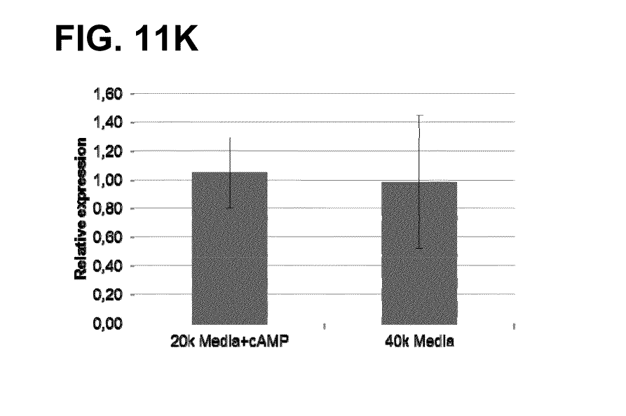

Proliferation of RPE cells is associated with a loss of the differentiated epithelial morphology instead of which cells become elongated and fibroblastic in appearance. This apparent ‘de-differentiation’ is followed by a phase of ‘re-differentiation’ where a confluent monolayer of cells take up the characteristic phenotype of cuboidal-shaped, pigmented RPE cells (Vugler et Al., Exp Neurol. 2008 December; 214(2):347-61). This de-differentiation re-differentiation paradigm, which occurs during expansion, has been described as an Epithelial-Mesenchymal Transition (EMT) followed by a Mesenchymal-Epithelial Transition (MET) (Tamiya et Al., IOVS, May 2010, Vol. 51, No. 5) (FIG. 11 A).

a) Expansion in the Presence of cAMP or Agents Increasing the Intracellular Concentration of cAMP Increase RPE Cells Yield and Maturity

RPE cells generated by spontaneous differentiation were seeded at 40000 cells/cm² in media alone or 20000 cells/cm² in media+0.5 mM cAMP. Media was changed thrice a week. Expression of the proliferation marker Ki67 was measured by immunocytochemistry at Day 15 and an increase in expression of Ki67 in cells seeded in the presence of cAMP was observed. On day 35, cells were fixed and nuclei were stained using Hoescht stain. The number of stained nuclei is equivalent to cell number. An increase in cell number was observed upon cAMP supplementation in cells seeded at a density of 20000 cells/cm² and this increase was equivalent to the number of cells obtained with a seeding density of 40000 cells/cm² in media alone (FIG. 11 B). This indicates that there is a doubling of yield by incorporating cAMP in the culture. Cells were also immunostained for PMEL17, an RPE marker. There was an increase in PMEL17 expression when cells were supplemented with cAMP and seeded at 20000 cells/cm² and this increase was similar to the level seen when cells were seeded at a higher density of 40000 cells/cm² (FIG. 11 C). This shows that the presence of cAMP during the expansion step increases the expression of the RPE markers thereby indicating increased maturity.

Furthermore, other chemical agents that increase intracellular concentration of cAMP e.g Forskolin, an activator of Adenylate Cyclase, also have similar effect to cAMP in terms of increasing cell yield and PMEL17 expression. RPE cells generated by spontaneous differentiation were seeded at 40000 cells/cm² in media alone or 20000 cells/cm² in media comprising 10 μM Forskolin. Media was changed thrice a week and cells were immunostained at Day 14. There was an increase in PMEL17 expression in the presence of Forskolin similar to the effect seen with cAMP (FIG. 11D)

### b) Whole Genome Transcript Profiling

In order to gain further understanding of the effect of cAMP on RPE cells expansion, a timecourse was set up where cells were seeded at a density of 10000 cells/cm² and 20000 cells/cm² in media alone or media supplemented with 0.5 mM cAMP. These were compared to RPE cells seeded at 40000 cells/cm² in media alone. Media was changed thrice a week. Samples were collected at D3, D15 and D35 post seeded and whole genome transcript analysis was performed in triplicate. It was seen that expression of RPE markers TYR, TYRP1, MITF, RPE65, BEST1 and MERTK was similar between cells seeded at a lower density but supplemented with cAMP as compared to those seeded at higher density but in media alone at all timepoints tested.

c) EdU Incorporation in cAMP Treated RPE

In addition to performing immunocytochemistry for Ki67, EdU incorporation in cells was used as an additional assay to measure proliferation of RPE cells in the presence of cAMP. Ki67 is expressed during all active phases of the cell cycle (G1, S, G2, and mitosis), but absent from resting cells (G0). However, the biological function of Ki67 is still largely unknown and it is unclear whether all cells expressing Ki67 complete mitosis. A complementary technique to measure proliferation is to measure the incorporation of Thymidine analogues such as EdU into the DNA which facilitates the identification of cells that have progressed through the S phase of the cell cycle during the EdU-labeling period.

RPE obtained by spontaneous differentiation of hESC cells were seeded at a density of 38000 cells/cm² and maintained in the presence or absence of 0.5 mM cAMP for a period of 8 weeks. EdU incorporation was measured at the following timepoints: Day 2, Day 3, Day 5, Day 7, Day 14, Day 21, Day 56 post seeding. Results were expressed as percentage of cells staining positive for EdU. An increase in % EdU was seen at timepoints of Day 7, Day 14 and Day 21 in cells treated with cAMP indicating that cAMP increased proliferation at these stages of RPE expansion (FIG. 11E). Quantification of cell number was extrapolated from the number of Hoescht positive nuclei imaged per frame. Each image frame captured had a size of 0.0645×0.0645 mm and the total surface area of the well was 6 mm². Therefore, the total cell number in the well was approximately equal to the number of Hoescht positive nuclei per image multiplied by a factor of 6/(0.0645×0.0645) which equals 1442.2. An increase in total cell number was observed upon addition of cAMP indicating that increased proliferation due to addition of cAMP resulted in an increase in number of RPE (FIG. 11F).

d) Dose of cAMP

RPE were seeded at a density of 20000 cells/cm² and treated with a range of cAMP concentrations: 500 μM, 50 μM, 5 μM, 0.5 μM and 0.05 μM for a period of 14 days. Controls were setup which included cells seeded at 40000 cells/cm² and 20000 cells/cm² in media alone. At the end of 14 days, cells were fixed and immunocytochemistry was performed to measure expression of Ki67, a marker of proliferation and PMEL17, a marker of RPE identity and purity. Nuclei were counterstained with the nuclear dye Hoescht.

A dose of 500 μM cAMP induced the expression of Ki67 in cells seeded at 20000 cells/cm² to a level similar to that of RPE seeded at double the density of 40000 cells/cm² in media alone (FIG. 11G). Furthermore, there was an increase in PMEL17 expression upon treatment with a dose of 500 μM cAMP. Without cAMP treatment, cells seeded at 20000 cells/cm² had low expression of PMEL17 (FIG. 11H).

This data show that a dose higher than 50 μM is sufficient to induce an effect of cAMP on proliferation and development of the RPE phenotype. Preferably a dose of 500 μM or higher can be used to induce proliferation of RPE cells.

e) Equivalence of RPE Patches Obtained after Expanding RPE at a Density of Either 20000 Cells/Cm² in the Presence of cAMP or 40000 Cells/Cm² in Media Alone.

A suspension of RPE obtained by spontaneous differentiation was seeded at a density of either 20000 cells/cm² or 40000 cells/cm² in 48 well format. The cells seeded at 20000 cells/cm² were treated with 500 μM cAMP whereas the cells seeded at 40000 cells/cm² were maintained in media alone for a period of 10 weeks. At the end of the period in expansion, cells from both conditions were lifted using Accutase and used to seed Transwells® at a density of 116000 cells/Transwell®. The Transwells® were maintained in culture for a period of 5 weeks in media alone. Spent media was collected weekly to quantify the levels of VEGF and PEDF in both conditions. At the end of the culture period, patches were cut and immunostained for the RPE marker ZO1. The outer region of the Transwell® was used for qPCR based analysis of gene expression for a panel of RPE markers.

At the end of expansion, we observed that cells from both expansion conditions had a similar morphology and showed the presence of characteristic pigmented, cobblestoned cells. Level of VEGF and PEDF secretion were quantified during the Transwell® culture and comparable VEGF:PEDF ratios were obtained from both sets of Transwells®, irrespective of whether they were obtained from cultures expanded at a density of 20000 cells/cm² in the presence of cAMP or 40000 cell/cm² in media. In terms of gene expression, we observed comparable expression of RPE genes (Mitf, Silv, Tyr) from the Transwells® set up from the two expansion conditions (FIGS. 11I, 11J and 11K). Furthermore, the protein expression of the RPE marker ZO-1 was comparable between the two conditions.

In summary, the data shows that there is no difference between RPE cultured on Transwells® expanded at 40000 cells/cm² in media or at half the seeding density i.e 20000 cells/cm² in the presence of cAMP.

### f) Expansion in the Presence of SMAD Inhibitors Increase RPE Cells Proliferation

1. Small Molecule Inhibitors of TGFβ Receptors (TGFBR) Increase RPE Proliferation and Expression of RPE Markers

TGFBR inhibitors listed in table 2 were investigated for their effect on RPE proliferation and expression of RPE markers.

TABLE 2

| Compound | | | |
|---|---|---|---|
| Number | Structure | Name | Reference |
| 1 | 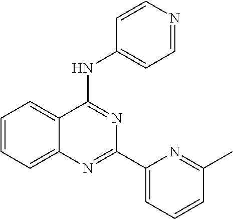 [SMILES: Cc1cccc(-c2nc(Nc3ccncc3)c3ccccc3n2)n1] | 2-(6-methylpyridin-2-yl)-N- (pyridin-4-yl)quinazolin-4- amine | Bioorganic & Medicinal Chemistry Letters (2009), 19(8), 2277-2281 |
| 2 | 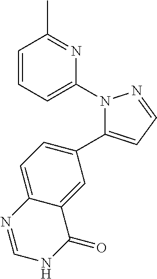 [SMILES: Cc1cccc(-n2nccc2-c2ccc3nc[nH]c(=O)c3c2)n1] | 6-(1-(6-methylpyridin-2-yl)-1H- pyrazol-5-yl)quinazolin-4(3H)- one | Bioorganic & Medicinal Chemistry Letters (2012), 22(10), 3392-3397 |
| 4 | 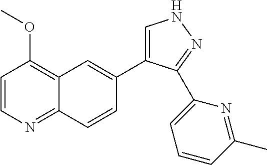 [SMILES: COc1ccnc2ccc(-c3c[nH]nc3-c3cccc(C)n3)cc12] | 4-methoxy-6-(3-(6- methylpyridin-2-yl)-1H-pyrazol- 4-yl)quinoline | WO200426306 |

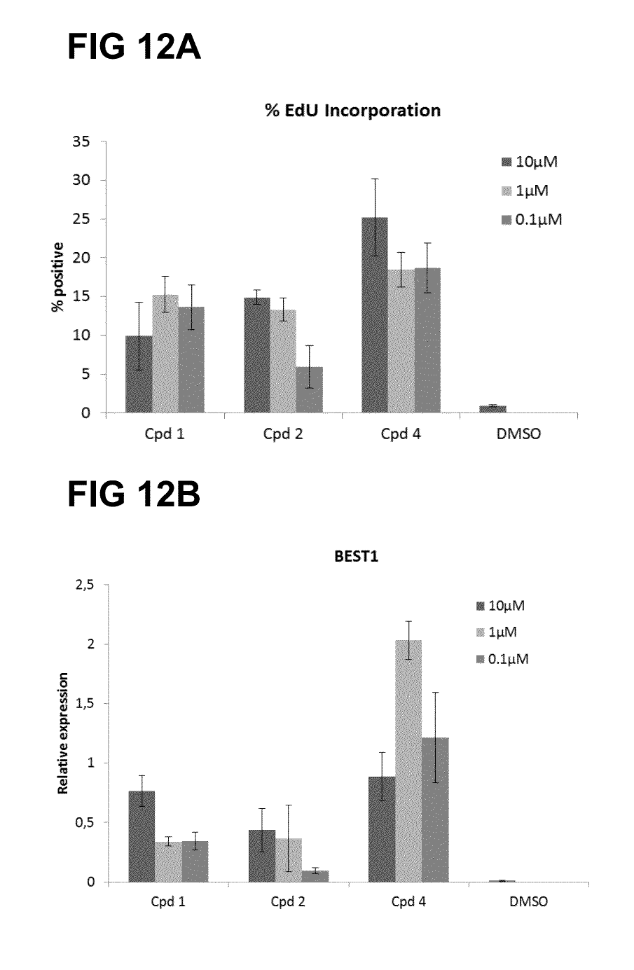

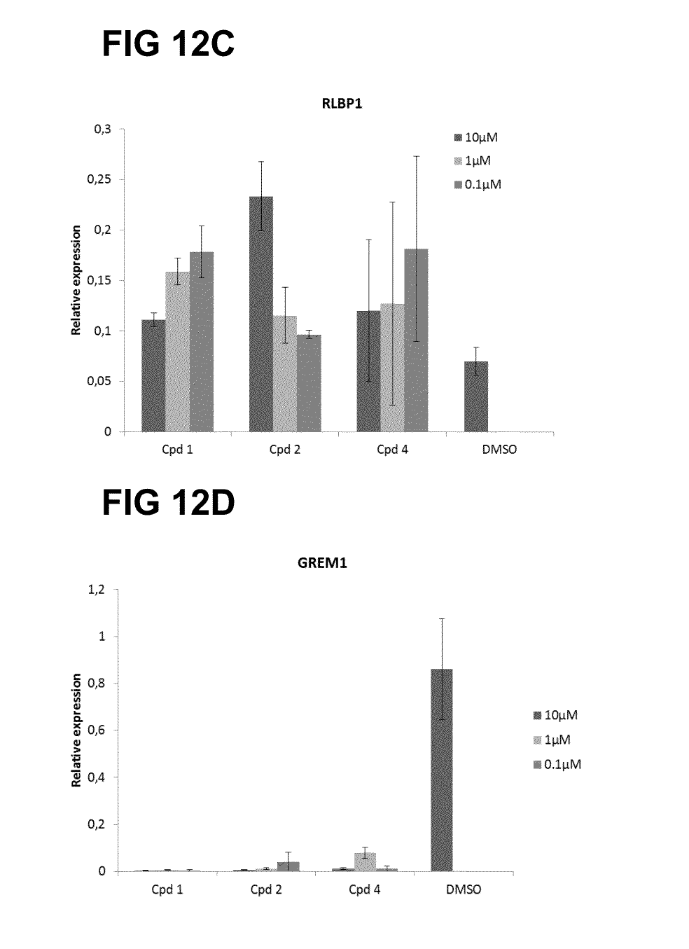

Compounds were added to RPE obtained from Shef-1 hESC cells as disclosed in example 10a seeded at a density of 2500 cells/cm² at a concentration of 10 μM, 1 μM and 0.1 μM. Compounds were maintained in the media for a period of 10 days. Proliferation was assessed by exposing the cells to 10 μM EdU for a period of 4 hours after which cells were fixed and EdU incorporated was detected using the Click-iT® EdU (Invitrogen, Catalogue# C10337) kit following manufacturer's recommendations. An increase in proliferation compared to vehicle treatment was observed upon treatment with all 3 compounds (see FIG. 12A). In order to test if increased proliferation caused by TGFBR inhibitors affected attainment of RPE phenotype, qPCR was carried out to measure the transcript levels of RPE markers Best1 and RIbp1. An increase in expression of RPE markers was observed upon compound treatment (see FIGS. 12B and 12C). We also checked the level of Grem1, a marker of de-differentiated RPE which was found to be lower in compound treated samples (see FIG. 12D).

This data shows that inhibition of SMAD signaling by TGFBR inhibitors increases proliferation and achievement of the RPE phenotype.

2. Antibody-Based Inhibition of SMAD Signaling Increases RPE Proliferation and Expression of RPE Markers

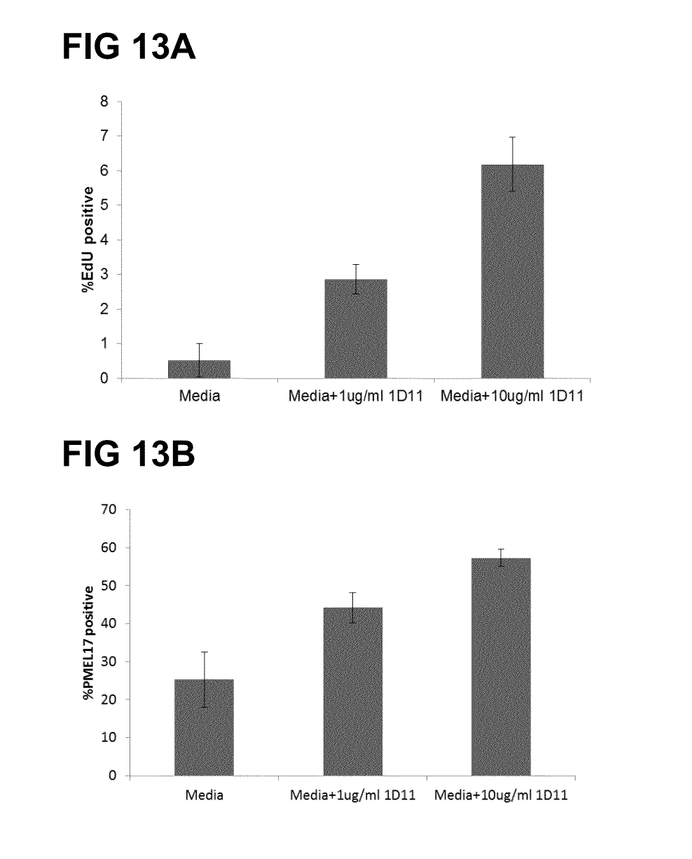

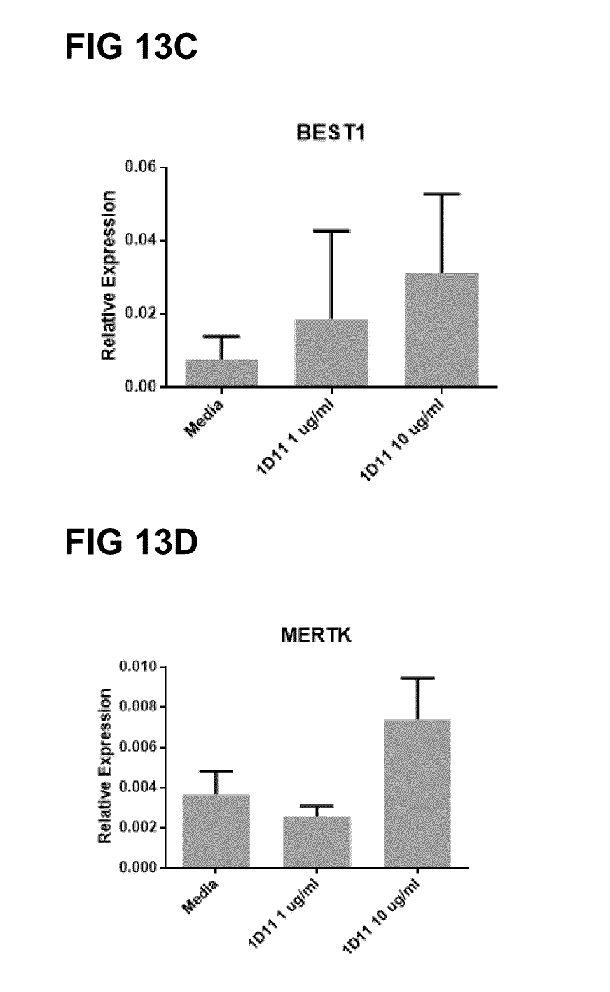

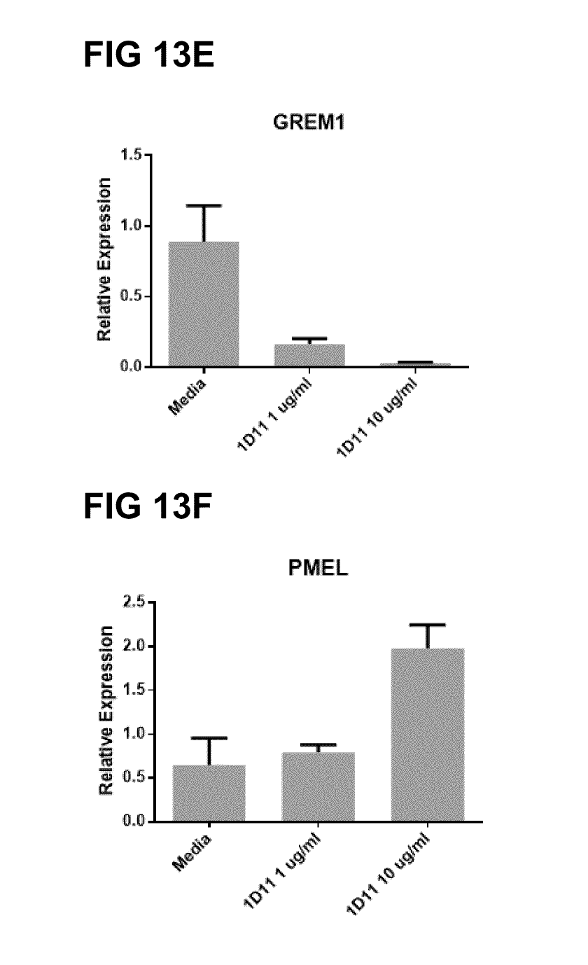

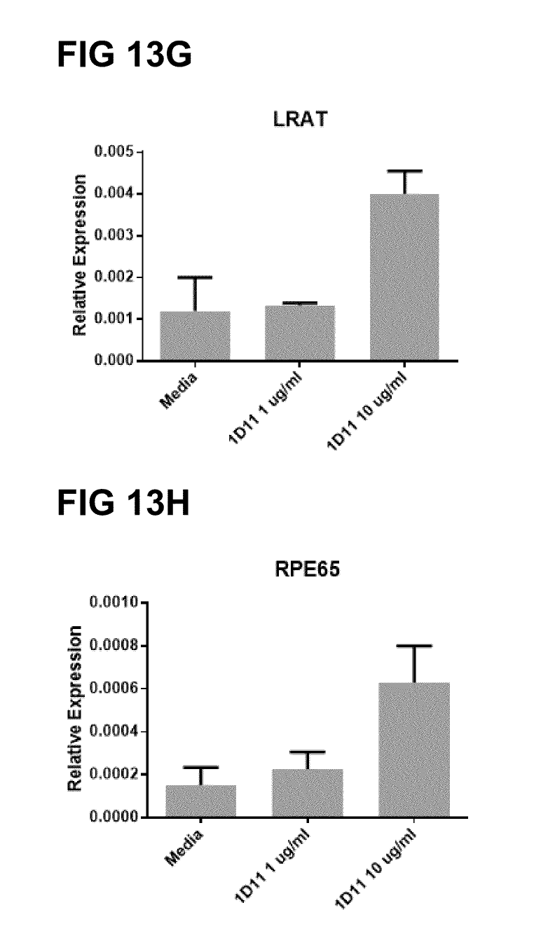

As an alternative means to inhibit SMAD signaling, a neutralizing antibody against TGFβ1 and TGFβ2 ligands known as 1D11 was used (The Journal of Immunology, Vol. 142, 1536-1541, No. 5. March 1989). RPE obtained from Shef-1 hESC cells as disclosed in example 10a were seeded at a density of 5000 cells/cm² and antibody 1D11 was added to the media at a concentration of 1 μg/ml and 10 μg/ml. Antibody was maintained in the media for a period of 14 days. Proliferation was assessed by exposing the cells to 10 μM EdU for a period of 4 hours after which cells were fixed and EdU incorporated was detected using Click chemistry following manufacturer's recommendations. An increase in proliferation compared to vehicle treatment was observed upon treatment with the neutralizing antibody in a dose-dependent manner (FIG. 13A). This showed that inhibition of SMAD signaling in RPE by an antibody inhibiting TGFβ1 and TGFβ2 increases RPE proliferation.

In order to test if increased proliferation caused by inhibition of TGFβ affected attainment of RPE phenotype, the level of RPE markers was checked by immunostaining and qPCR. An increase in expression of PMEL17 was seen at both the protein (see FIG. 13B) and transcript level along with an increase in transcript levels of a panel of other RPE markers as well as a decrease in level of the de-differentiated RPE marker GREM1 (see FIGS. 13C to 13H).

This data shows that inhibition of SMAD signaling by an antibody inhibiting TGFβ1 and TGFβ2 pathways increases proliferation and achievement of the RPE phenotype.

### Example 13

### Purification of RPE Cells

### a) Screen to Identify Cell Surface Marker Expression

Cells were obtained from Shef1.3 hESC by following the directed differentiation protocol with early replating. Cells were cultured up to day 9 on Matrigel and replated onto Cellstart (Replate 1) where they were cultured for 19 days followed by replating onto Cellstart (Replate 2) where they were cultured for 15 days before being used for this experiment. Cells were plated at a density of 100000 cells/cm² onto 384 well plates coated with Matrigel. Cells were cultured for 7 days before performing a screen for cell surface protein expression using the BD Lyoplate Human Cell Surface Marker Screening Panel (BD Biosciences, Cat#560747). Manufacturer's recommendations were followed for screening cells by bioimaging. Images of cell staining were analysed for positive expression of markers. Cells were also stained for PMEL17, CRALBP and ZO1 which are RPE markers to confirm RPE identity. CD59 was identified to be expressed in RPE cells above the isotype background.

b) Flow Cytometry for CD59 on Samples from the Directed Differentiation Process with Early Replating

CD59 expression was quantified using Flow cytometry. The following samples of cells from the Directed Differentiation protocol were prepared for analysis:

1/ Shef1 hESC (Day 0),
2/ Day 6 (1 μM LDN193189 and 10 μM SB-431542 from day 2 to Day 6),
3/ Day 9 (LDN/SB minus BMP4/7): 1 μM LDN193189 and 10 μM SB-431542 from day 2 to day 6 and medium without BMP4/7 from Day 6 to day 9)
4/ Day 9 (LDNSB plus BMP4/7): 1 μM LDN193189 and 10 μM SB-431542 from day 2 to day 6 and 100 ng/ml BMP4/7 from Day 6 to day 9)
5/ Two replicates of RPE samples obtained after Replete 2: LDN/SB from day 2 to day 6, BMP4/7 from Day 6 to day 9, replated at D9 in the presence of Activin A for a period of 2 weeks and then in media alone for a period of 3 months.

All samples were collected using Accutase. Cells were stained with a Live/Dead dye using the Live/Dead fixable dead cell stain kit fluorescent in the green (FL2) channel (Invitrogen, Cat# L23101). Cells were fixed with 1% PFA and washed with PBS(−/−) three times. Centrifugation was performed at 300×g for 5 minutes. Cells were resuspended to approximately 1×10⁶ cells/100 L in PBS(−/−) +2% BSA. Cells were stained for CD59 using the PE Mouse Anti-Human CD59 antibody (BD Pharmingen, Cat#560953). 20 μL antibody was used per test in a 100 μL experimental sample. Samples were incubated for 30 minutes protected from light at room temperature. Samples were washed 2 times before being resuspended in 150 μL PBS(−/−) +2% BSA for analysis on the Accuri C6 Flow cytometer. Negative controls consisting of unstained cells and cells stained with the isotype control (PE Mouse IgG2a, eBioscience Cat#12-4724-41) were also performed. Flow cytometry analysis was performed by gating out the debris and doublets and only selecting the population staining positive for the live cell stain. The results from this analysis are shown in table 3.

TABLE 3
percentage of positive CD59 staining by Flow cytometry in samples
obtained from the Directed Differentiation timecourse

| | | | Day 9 | Day 9 | | |
|---|---|---|---|---|---|---|
| | | | (LDNSB | (LDNSB | RPE after | |
| | | | minus | plus | 2nd replate | |
| Sample | Day 0 | Day 6 | BMP4/7) | BMP4/7) | RPE1 | RPE2 |
| Unstained | 6.6 | 0.1 | 4.2 | 0.5 | 0.9 | 0 |
| Isotype PE | 5.8 | 0.1 | 4.2 | 0.5 | 1.1 | 1.2 |
| CD59 PE | 6.3 | 0 | 4.9 | 0.7 | 99 | 99.4 |

This shows that CD59 is not expressed at the early timepoints of the directed differentiation protocol before replating and is only expressed in mature RPE obtained after second replating. Therefore, sorting for cells expressing CD59 may be a means to enrich for mature RPE and remove any RPE progenitors or other CD-59-negative cells that may possibly be present as residual contaminating cells in the final RPE culture.

c) Spiking Experiment with Shef1 hESC and RPE Obtained after Replate 2 of Directed Differentiation Protocol

In order to show specificity of CD59 expression on RPE, a spiking experiment was performed. Shef1 hESC and RPE obtained after Replate 2 of the directed differentiation protocol with early replating were collected using Accutase. Cells were stained with a Live/Dead dye using the Live/Dead fixable dead cell stain kit fluorescent in the Far-Red (FL4) channel (Invitrogen, Cat# L10120) before being fixed with 1% PFA and resuspended to the same concentration in PBS(−/−) +2% BSA. The following ratios of hESC and RPE were mixed together to give a final volume of 100 μL: 100% RPE+0% hESC; 75% RPE+25% hESC; 50% RPE+50% hESC; 25% RPE+75% hESC; 0% RPE+100% hESC. Flow cytometry was performed on all samples for CD59 and TRA-1-60, a marker of pluripotent ES cells. Negative controls consisting of unstained cells and cells stained with the appropriate isotype controls were also performed. Samples were analysed on a the Accuri C6 Flow cytometer. Flow cytometry analysis was performed by gating out the debris and doublets and only selecting the population staining positive for the live cell stain. The results from this analysis are shown in Tables 4 and 5.

TABLE 4
% CD59 positive staining by Flow cytometry

| | |
|---|---|
| Spiked | Detected |

| CD59 | Shef (%) | RPE (%) | % |
|---|---|---|---|
| unstained | 0 | 100 | 0 |
| isotype | 0 | 100 | 0 |
| | 0 | 100 | 94.5 |
| | 75 | 25 | 30.5 |
| | 50 | 50 | 58.6 |
| | 25 | 75 | 76.4 |
| | 100 | 0 | 2.1 |

TABLE 5
% TRA-1-60 positive staining by Flow cytometry

| | |
|---|---|
| Spiked | Detected |

| Tra160 | RPE (%) | Shef (%) | % |
|---|---|---|---|
| unstained | 0 | 100 | 0.1 |
| isotype | 0 | 100 | 1 |
| | 0 | 100 | 73.4 |
| | 75 | 25 | 18.5 |
| | 50 | 50 | 34.1 |
| | 25 | 75 | 54.4 |
| | 100 | 0 | 0.5 |

These results show that the level of detected CD59 correlates to the proportion of RPE present in a sample and that the antibody is able to discriminate against other non-RPE cells present in a sample. Furthermore, the proportion of non-RPE hESC cells spiked into the sample correlates to the % TRA-1-60 detected. Therefore, sorting for cells expressing CD59 may be a means to enrich for mature RPE and remove any hESC or RPE progenitors that may possibly be present as residual contaminating cells in the final RPE culture.

d) Use of Flow Cytometry to Sort CD59 Positive RPE from a Mixed Population of ESC and RPE Cells

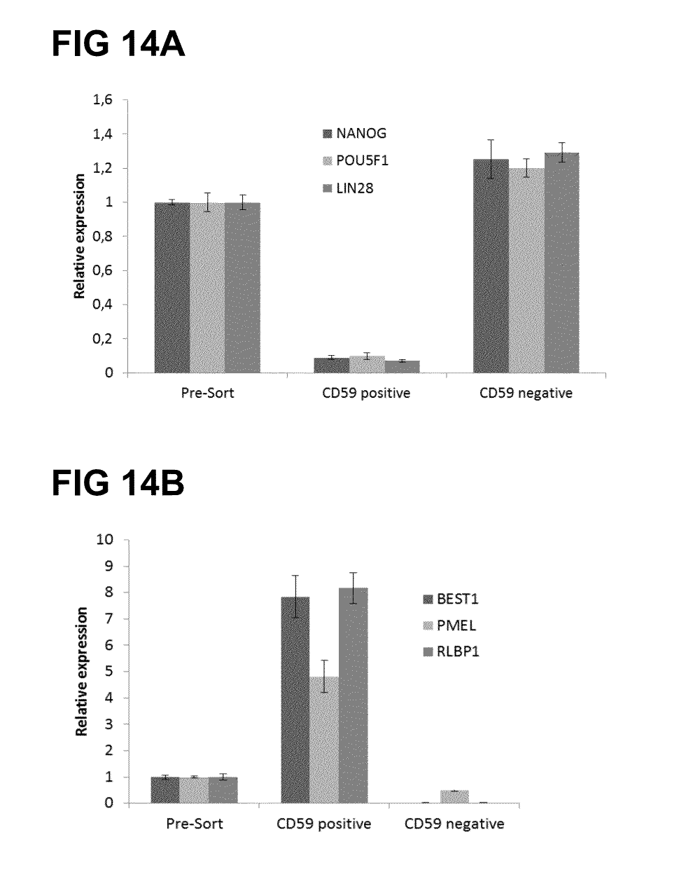

In order to show that it is possible to enrich RPE from a mixed population using CD59 sorting, an equal number of hESC and RPE cells (obtained after Early Replate 2) were mixed together. A sample from this mixture was kept separate as the Pre-sorted population. The remaining mixture was stained with PE Mouse Anti-Human CD59 antibody (BD Pharmingen, Cat#560953). 20 μL antibody was used per test in a 100 μL experimental sample containing 1×10⁶ cells. Samples were incubated for 30 minutes protected from light at room temperature. Samples were washed 2 times before being resuspended at a density of 1×10⁶ cells per ml of PBS(−/−) +2% BSA. CD59 positive cells were sorted on an inFlux v7 cytometer and collected separately from CD59 negative population. RNA was extracted from the Pre-sorted, CD59 positive and CD59 negative fractions. qPCR was used to check expression of a panel of ES and RPE markers. This showed that the CD59 positive fraction was enriched with RPE markers Best1, Silv, RIbp1 (see FIG. 14B) and the CD59 negative fraction was enriched with the ES markers Nanog, Pou5f1 and Lin28 (See FIG. 14A). This shows that Flow sorting for CD59 can enrich RPE cells from a mixed population and remove non-RPE cell types.

### Example 14

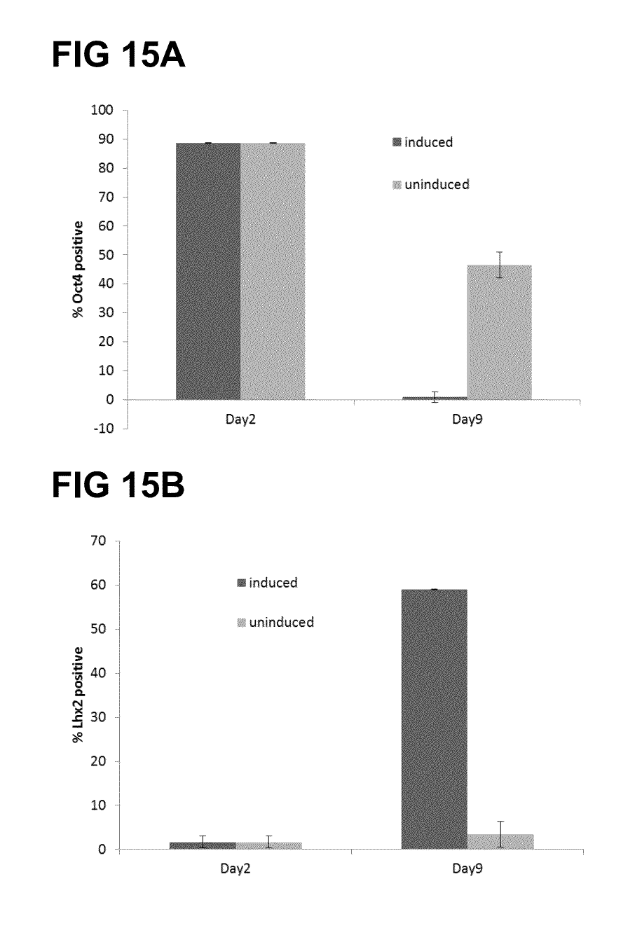

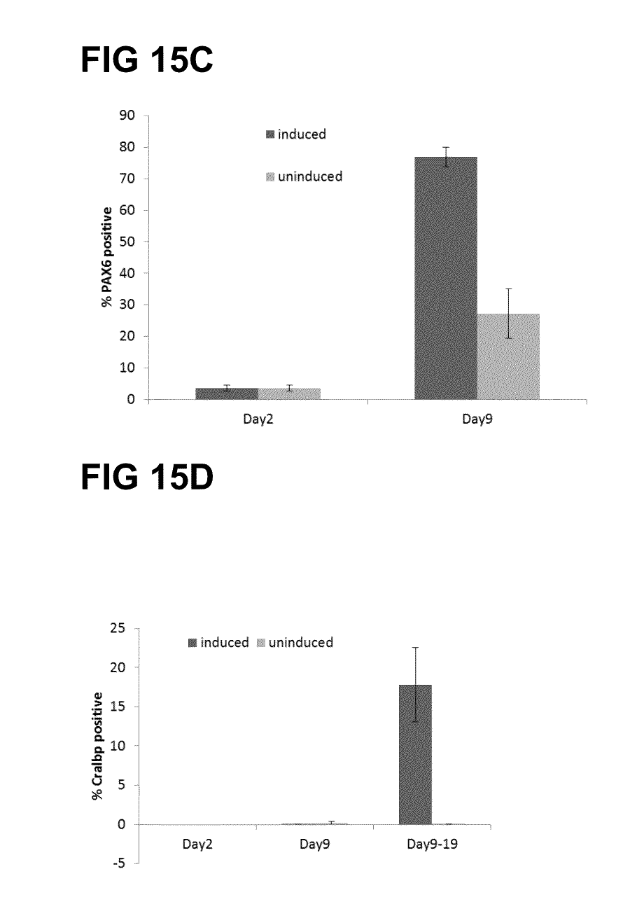

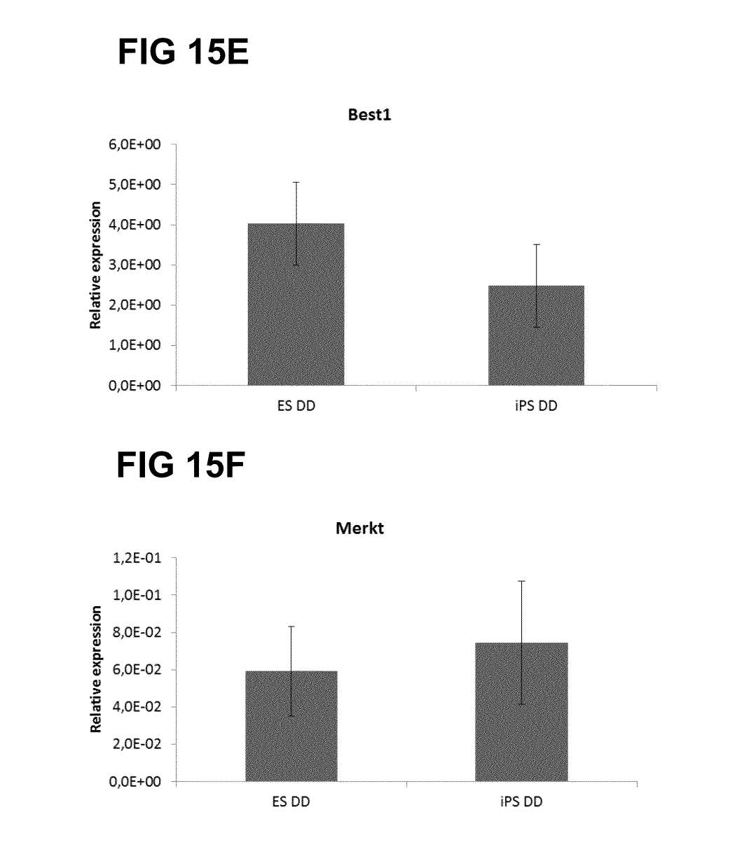

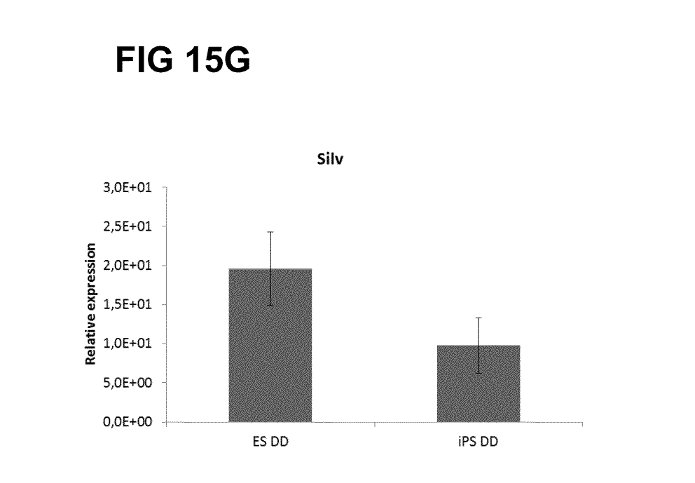

The directed differentiation protocol was performed on induced pluripotent cells (iPSCs). IPSCs were generated from erythroblasts obtained from healthy volunteers and reprogrammed using the CytoTune-iPS Reprogramming kit (Life Technologies, A13780-01/02). IPSCs were seeded in E8 medium at a density of 240000 cells/cm² and differentiated to Day 9-19 of the directed differentiation protocol with early replating. Induced cells refer to cells treated with LDN193189/SB-431542 from Day 2 to Day 6 followed by BMP4/7 from Day 6 to Day 9. Uninduced cells are maintained without exposure to both LDN193189/SB-431542 and BMP4/7. Immunostaining was performed for markers of interest. As seen in FIGS. 15A to 15D, induced iPSC downregulated OCT4 at Day 9 and upregulated PAX6 and LHX2 similar to induced hESC. Following replating at Day 9 in the presence of Activin A, iPSCs upregulated the RPE marker CRALBP. Following the second replete step at Day 9-19 and culturing for a period of 45 days, iPSCs derived RPE expressed a panel of RPE markers to similar levels seen in RPE derived by directed differentiation from ES cells as obtained by the protocol of example 8 (see FIGS. 15E, 15F and 15G). Therefore, these results demonstrate that the directed differentiation protocol is transferable to IPSCs for the generation of RPE.

The following methods were used in the above examples:

### Immunocytochemistry:

Immunocytochemistry was carried out in 96-well or 384-well format. Media was aspirated and 50 μL 4% paraformaldehyde (PFA) was added to each well and incubated for 35 minutes at room temperature. PFA was aspirated and cells washed 3×100 uL PBS(+/+). Cells were incubated for 1 hour at room temperature in the dark in blocking buffer (PBS(+/+)/5% normal donkey serum (NDS)/0.3% TritonX100). 1° antibodies were made up in PBS(+/+)/1% normal donkey serum (NDS)/0.3% TritonX100. 60 μL 1° antibody solution was added to each well and incubated for 1 hour at room temperature in the dark. Solution was aspirated and cells washed 3×100 uL PBS(+/+). 2° antibodies were made up in PBS(+/+)/1% normal donkey serum (NDS)/0.3% TritonX100. 60 uL 2° antibody solution was added to each well and incubated for 1 hour at room temperature in the dark. Solution was aspirated and cells washed 3×100 uL PBS(+/+). Hoechst 33342 solution was diluted 1:5000 (2 μg/mL final concentration) in PBS(+/+) and 50 μL added to each well and incubated for at least 6 minutes at room temperature in the dark. Solution was aspirated and cells washed 1×PBS(+/+), then 100 μL PBS(+/+) added to each well and plates sealed and stored in the fridge until imaged. Images were captured on the IXM MetaExpress Platform at 10×, 20× magnification.

| | | | | Catalogue | |
|---|---|---|---|---|---|
| Antibody | 1° or 2° | Species | Supplier | number | Dilution |
| Anti-CRALBP | 1° | Mouse | Pierce | MA1-813 | 1:200 |
| Anti-PMEL17 | 1° | Mouse | Dako | M0634 | 1:35 |
| Anti-Z01 | 1° | Rabbit | Invitrogen | 18-7430 | 1:200 |
| Anti-MERTK | 1° | Rabbit | Abeam | Ab52968 | 1:50 |
| Anti-BEST1 | 1° | Mouse | Millipore | MAB5466 | 1:100 |
| 488 nm anti- | 2° | Donkey | Life | A21202 | 1:1000 |
| mouse | | | Technologies | | |
| 594 nm anti- | 2° | Donkey | Life | A21203 | 1:1000 |
| mouse | | | Technologies | | |
| 488 nm anti- | 2° | Donkey | Life | A21206 | 1:1000 |
| rabbit | | | Technologies | | |
| 594 nm anti- | 2° | Donkey | Life | A21207 | 1:1000 |
| rabbit | | | Technologies | | |

### Molecular Biology Techniques:

### RNA Extraction

Media was aspirated and cells were washed with 100 μL PBS(−/−). 100 μL Buffer RLT (1% 2-mercaptoethanol) was added to each well and the pipetted up and down, before transferring the lysate to a 2 mL tube containing a further 250 μL Buffer RLT (1% 2-mercaptoethanol). Samples were stored at −80° C. until processing. RNA was extracted using the RNeasy micro kit (Qiagen), including on column DNase digest on the Qiacube as per the manufacturer's protocol. RNA was eluted with 14 μL RNase-free water.

cDNA Synthesis

cDNA was synthesised using the Applied Biosystems High Capacity RNA-to-cDNA kit:

| | 1x Reaction Mix |
|---|---|
| 2x RT Buffer | 10 |
| 20x RT Enzyme | 1 |
| RNA | 4 |
| Nuclease-free H2O | 5 |
| Total | 20 |

Mastermix (16 μL) was aliquoted into wells of a 96-well plate and 4 uL RNA added to each well. Nuclease-free water was added to one well to act as a no template control. The plate was then centrifuged at 1000 rpm for 1 minute to collect, and the plate transferred to a thermal cycler and cDNA synthesised using the following protocol:

| Step | Temperature | Time |
|---|---|---|
| 1 | 37° C. | 60 minutes |
| 2 | 95° C. | 5 minutes |
| 3 | 4° C. | Hold |

cDNA samples were diluted with 80 uL nuclease-free water and stored at −20° C. until further use.

### Quantitative PCR

qPCR mastermixes were made up for each assay as follows, using the Applied Biosystems Taqman Gene Expression Mastermix:

| | 1x Reaction Mix |
|---|---|
| 2x Taqman Gene Expression | 10 |
| Mastermix | |
| Primer/Probe mix | 1 |
| Nuclease-Free water | 7 |
| cDNA/Template | 2 |
| Total | 20 |

Matermix (18 uL) was aliquoted into wells for a 96-well plate and 2 uL cDNA (or control) added to each well. Controls were no template control from the cDNA synthesis, water, and spontaneously differentiated RPE cDNA. Each sample was run in duplicate. The plate was then centrifuged at 1000 rpm for 1 minute to collect, and the plate transferred to a thermal cycler and the qPCR assay run using the following protocol:

| Step | Temperature | Time |
|---|---|---|
| 1 | 50° C. | 2 minutes |
| 2 | 95° C. | 10 minutes |
| 3 | 95° C. | 15 seconds |
| 4 | 60° C. (data collection) | 1 minute |
| 5 | Go to step 3 49x | |
| 6 | 4°C. | 2 minutes |

Data was exported to Microsoft Excel and analysed using the 2̂-DCT method.

List of genes tested by qPCR in Example 10:

| | | Taqman assay |
|---|---|---|
| Gene | Category | ID |
| GAPDH | Reference | Hs99999905_m1 |
| HPRT1 | Reference | Hs99999909_m1 |
| IPO8 | Reference | Hs00183533_m1 |
| LHX2 | Eye Field | Hs00180351_m1 |
| SIX3 | Eye Field | Hs00193667_m1 |
| TBX5 | Eye Field | Hs00361155_m1 |
| OTX2 | Early | Hs00222238_m1 |
| | RPE/Neuroectoderm | |
| PAX6 | Early | Hs01088112_m1 |
| | RPE/Neuroectoderm | |
| BEST1 | RPE | Hs00188249_m1 |
| MERTK | RPE | Hs00179024_m1 |
| MITF | RPE | Hs01117294_m1 |
| RLBP1 | RPE | Hs00165632_m1 |
| RPE65 | RPE | Hs00165642_m1 |
| SILV | RPE | Hs00173854_m1 |
| TYR | RPE | Hs01099965_m1 |
| TYRP1 | RPE | Hs00167051_m1 |
| TJP1 | Tight Junctions | Hs00268480_m1 |
| CRX | Retinal | Hs00230899_m1 |
| RX | Retinal | Hs00429459_m1 |
| Ki67 | Proliferation | Hs01032443_m1 |
| THBS1 | Cell Surface Interactions | Hs00962908_m1 |
| ITGAV | Cell Surface Interactions | Hs00233808_m1 |
| GREM1 | Epithelial-Mesenchymal | Hs00171951_m1 |
| | Transition | |
| FOXC2 | Epithelial-Mesenchymal | Hs00270951_s1 |
| | Transition | |
| CPA4 | Epithelial-Mesenchymal | Hs00275311_m1 |
| | Transition | |
| CDKN1B | Epithelial-Mesenchymal | Hs00153277_m1 |
| | Transition | |
| RRS1 | Epithelial-Mesenchymal | Hs00534971_s1 |
| | Transition | |
| BMP7 | Epithelial-Mesenchymal | Hs00233476_m1 |
| | Transition | |
| SFRP5 | Epithelial-Mesenchymal | Hs00169366_m1 |
| | Transition | |
| FRZB | Epithelial-Mesenchymal | Hs00173503_m1 |
| | Transition | |
| DCT | Epithelial-Mesenchymal | Hs01098278_m1 |
| | Transition | |
| CDH1 | Epithelial-Mesenchymal | Hs01023894_m1 |
| | Transition | |
| VEGF A | Secreted Factor | Hs00900055_m1 |
| PEDF | Secreted Factor | Hs01106937_m1 |
| SFTPD | Secreted Factor | Hs00358340_m1 |
| ASIP | Secreted Factor | Hs00181770_m1 |
| IGFBP1 | Secreted Factor | Hs00426285_m1 |
| C3 | Secreted Factor | Hs00163811_m1 |
| LIF | Secreted Factor | Hs00171455_m1 |
| IL8 | Secreted Factor/ | Hs00174103_m1 |
| | Immunomodulation | |
| CCL2 | Secreted Factor/ | Hs00234140_m1 |
| | Immunomodulation | |
| HLA-A | Immunomodulation | Hs01058806_g1 |
| HLA- | Immunomodulation | Hs00185435_m1 |
| DMA | | |
| IL-10 | Immunomodulation | Hs00961622_m1 |
| IL-6 | Immunomodulation | Hs00174131_m1 |
| ATP1B1 | Ion Channels | Hs00426868_g1 |
| TRPM1 | Ion Channels | Hs00170127_m1 |
| TRPM3 | Ion Channels | Hs00257553_m1 |

1. A method for producing retinal pigment epithelial (RPE) cells comprising the steps of:
(a) culturing pluripotent cells in the presence of a first SMAD inhibitor and a second SMAD inhibitor;
(b) culturing the cells of step (a) in the presence of a BMP pathway activator and in the absence of the first and second SMAD inhibitors; and,
(c) replating the cells of step (b).

2. The method according to claim 1 wherein, in step (a), the cells are cultured as a monolayer.

3. The method according to claim 1 or 2 wherein, in step (b), the cells are cultured as a monolayer.

4. The method according to claim 1 wherein, in step (a), the cells are cultured in a suspension culture.

5. The method according to any one of claim 1, 2 or 4 wherein, in step (b), the cells are cultured in a suspension culture.

6. The method according to any one of claims 1 to 5, wherein the pluripotent cells are selected from embryonic stem cells or induced pluripotent stem cells.

7. The method according to any one of claims 1 to 6, wherein the pluripotent cells are human cells.

8. The method according to any one of claims 1 to 7, wherein the pluripotent cells are human embryonic stem cells.

9. The method according to any one of claims 1 to 7, wherein the pluripotent cells are human induced pluripotent stem cells.

10. The method according to any one of claims 1 to 9, wherein the pluripotent cells are obtained by means which do not require the destruction of a human embryo.

11. The method according to any one of claims 1 to 10 wherein the first SMAD inhibitor is an inhibitor of BMP type 1 receptor ALK2.

12. The method according to any one of claims 1 to 11 wherein the first SMAD inhibitor is an inhibitor of BMP type 1 receptors ALK2 and ALK3.

13. The method according to any one of claims 1 to 12 wherein the first SMAD inhibitor prevents Smad1, Smad5 and/or Smad8 phosphorylation.

14. The method according to any one of claims 1 to 13 wherein the first SMAD inhibitor is a dorsomorphin derivative.

15. The method according to any one of claims 1 to 13 wherein the first SMAD inhibitor is selected from dorsomorphin, noggin or chordin.

16. The method according to any one of claims 1 to 13 wherein the first SMAD inhibitor is 4-(6-(4-(piperazin-1-yl)phenyl)pyrazolo[1,5-a]pyrimidin-3-yl)quinoline (LDN193189) or a salt or hydrate thereof.

17. The method according to any one of claims 1 to 16 wherein, in step (a), the concentration of first SMAD inhibitor is between 0.5 nM and 10 μM.

18. The method according to any one of claims 1 to 17 wherein, in step (a), the concentration of first SMAD inhibitor is between 500 nM and 2 μM.

19. The method according to any one of claims 1 to 18 wherein, in step (a), the concentration of first SMAD inhibitor is about 1 μM.

20. The method according to any one of claims 1 to 19 wherein the second SMAD inhibitor is an inhibitor of ALK5.

21. The method according to any one of claims 1 to 20 wherein the second SMAD inhibitor is an inhibitor of ALK5 and ALK4.

22. The method according to any one of claims 1 to 21 wherein the second SMAD inhibitor is an inhibitor of ALK5 and ALK4 and ALK7.

23. The method according to any one of claims 1 to 20 wherein the second SMAD inhibitor is selected from:
4-(4-(benzo[d][1,3]dioxol-5-yl)-5-(pyridin-2-yl)-1H-imidazol-2-yl)benzamide;
2-methyl-5-(6-(m-tolyl)-1H-imidazo[1,2-a]imidazol-5-yl)-2H-benzo[d][1,2,3]triazole;
2-(6-methylpyridin-2-yl)-N-(pyridin-4-yl)quinazolin-4-amine;
2-(3-(6-methylpyridin-2-yl)-1H-pyrazol-4-yl)-1,5-naphthyridine;
4-(2-(6-methylpyridin-2-yl)-5,6-dihydro-4H-pyrrolo[1,2-b]pyrazol-3-yl)phenol;
2-(4-methyl-1-(6-methylpyridin-2-yl)-1H-pyrazol-5-yl)thieno[3,2-c]pyridine;
4-(5-(3,4-dihydroxyphenyl)-1-(2-hydroxyphenyl)-1H-pyrazol-3-yl)benzamide;
2-(5-chloro-2-fluorophenyl)-N-(pyridin-4-yl)pteridin-4-amine;
6-methyl-2-phenylthieno[2,3-d]pyrimidin-4(3H)-one;
3-(6-Methyl-2-pyridinyl)-N-phenyl-4-(4-quinolinyl)-1H-pyrazole-1-carbothioamide (A 83-01);
2-(5-Benzo[1,3]dioxol-5-yl-2-tert-butyl-3H-imidazol-4-yl)-6-methylpyridine (SB-505124);
7-(2-morpholinoethoxy)-4-(2-(pyridin-2-yl)-5,6-dihydro-4H-pyrrolo[1,2-b]pyrazol-3-yl)quinoline (LY2109761);
4-[3-(2-pyridinyl)-1H-pyrazol-4-yl]-quinoline (LY364947); or,
4-(4-(benzo[d][1,3]dioxol-5-yl)-5-(pyridin-2-yl)-1H-imidazol-2-yl)benzamide (SB-431542)
or a salt or hydrate thereof.

24. The method according to any one of claims 1 to 20, wherein the second SMAD inhibitor is 4-(4-(benzo[d][1,3]dioxol-5-yl)-5-(pyridin-2-yl)-1H-imidazol-2-yl)benzamide (SB-431542).

25. The method according to any one of claims 1 to 24 wherein, in step (a), the concentration of second SMAD inhibitor is between 0.5 nM and 100 μM.

26. The method according to any one of claims 1 to 25 wherein, in step (a), the concentration of second SMAD inhibitor is between 1 μM and 50 μM.

27. The method according to any one of claims 1 to 26 wherein, in step (a), the concentration of second SMAD inhibitor is about 10 μM.

28. The method according to any one of claims 1 to 27 wherein, in step (a), the pluripotent cells are cultured for at least 1 day.

29. The method according to any one of claims 1 to 28 wherein, in step (a), the pluripotent cells are cultured for at least 2 days.

30. The method according to any one of claims 1 to 29 wherein, in step (a), the pluripotent cells are cultured for between 2 and 10 days.

31. The method according to any one of claims 1 to 30 wherein, in step (a), the pluripotent cells are cultured for between 3 and 5 days.

32. The method according to any one of claims 1 to 31 wherein, in step (a), the pluripotent cells are cultured for about 4 days.

33. The method according to any one of claims 1 to 32 wherein, before step (a), the cells are cultured as a monolayer at an initial density of at least 1000 cells/cm².

34. The method according to any one of claims 1 to 33 wherein, before step (a), the cells are cultured as a monolayer at an initial density of between 100000 and 500000 cells/cm².

35. The method according to any one of claims 1 to 34 wherein the BMP pathway activator comprises a BMP.

36. The method according to any one of claims 1 to 35 wherein the BMP pathway activator comprises a BMP selected from BMP2, BMP3, BMP4, BMP6, BMP7, BMP8, BMP9, BMP10, BMP11 or BMP15.

37. The method according to any one of claims 1 to 36 wherein the BMP pathway activator is a BMP homodimer.

38. The method according to any one of claims 1 to 36 wherein the BMP pathway activator is a BMP heterodimer.

39. The method according to any one of claims 1 to 36 wherein the BMP pathway activator is a BMP2/6 heterodimer, a BMP4/7 heterodimer or a BMP3/8 heterodimer.

40. The method according to any one of claims 1 to 36 wherein the BMP pathway activator is a BMP4/7 heterodimer.

41. The method according to any one of claims 1 to 40 wherein, in step (b), the concentration of BMP pathway activator is between 1 ng/mL and 10 μg/mL.

42. The method according to any one of claims 1 to 41 wherein, in step (b), the concentration of BMP pathway activator is between 50 ng/mL and 500 ng/mL.

43. The method according to any one of claims 1 to 42 wherein, in step (b), the concentration of BMP pathway activator is about 100 ng/mL.

44. The method according to any one of claims 1 to 43 wherein, in step (b), said cells are cultured for at least 1 day.

45. The method according to any one of claims 1 to 44 wherein, in step (b), said cells are cultured for between 2 days and 20 days.

46. The method according to any one of claims 1 to 45 wherein, in step (b), said cells are cultured for about 3 days.

47. The method according to any one of claims 1 to 46 wherein, in step (c), said cells are replated at a density of at least 1000 cells/cm².

48. The method according to any one of claims 1 to 47 wherein, in step (c), said cells are replated at a density of between 100000 and 1000000 cells/cm².

49. The method according to any one of claims 1 to 48 wherein, in step (c), said cells are replated at a density of about 500000 cells/cm².

50. The method according to any one of claims 1 to 49 wherein, in step (c), said cells are replated on Matrigel®, fibronectin or Cellstart®.

51. The method according to any one of claims 1 to 50, wherein said method further comprises the following steps:
(d) culturing the replated cells of step (c) in the presence of an activin pathway activator;
(e) replating the cells of step (d); and,
(f) culturing the replated cells of step (e).

52. The method according to claim 51 wherein, in step (d), the cells are cultured for at least 1 day.

53. The method according to claim 51 or 52 wherein, in step (d), the cells are cultured for at least 3 days.

54. The method according to any one of claims 51 to 53 wherein, in step (d), the cells are cultured for between 3 and 20 days.

55. The method according to any one of claims 51 to 54 wherein, in step (d), the concentration of activin pathway activator is between 1 ng/mL and 10 μg/mL.

56. The method according to any one of claims 51 to 55 wherein, in step (d), the concentration of activin pathway activator is about 100 ng/mL.

57. The method according to any one of claims 51 to 56 wherein, in step (d), the activin pathway activator is activin A.

58. The method according to any one of claims 51 to 57 wherein, in step (d), the cells are cultured in the presence of cAMP.

59. The method according to claim 58 wherein, in step (d), the concentration of cAMP is about 0.5 mM.

60. The method according to any one of claims 51 to 59 wherein, in step (e), the cells are replated at a density of at least 1000 cells/cm².

61. The method according to any one of claims 51 to 60 wherein, in step (e), said cells are replated at a density of between 20000 and 500000 cells/cm².

62. The method according to any one of claims 51 to 61 wherein, in step (e), said cells are replated at a density of about 200000 cells/cm².

63. The method according to any one of claims 51 to 62 wherein, in step (e), said cells are replated on Matrigel®, fibronectin or Cellstart®.

64. The method according to any one of claims 51 to 63 wherein, in step (f), the cells are cultured for at least 5 days.

65. The method according to any one of claims 51 to 64 wherein, in step (f), the cells are cultured for at least 14 days.

66. The method according to any one of claims 51 to 65 wherein, in step (f), the cells are cultured for between 10 and 35 days.

67. The method according to any one of claims 51 to 66 wherein, in step (f), the cells are cultured for about 28 days.

68. The method according to any one of claims 51 to 67 wherein, in step (f), the cells are cultured in the presence of cAMP.

69. The method according to claim 68 wherein, in step (f), the concentration of cAMP is about 0.5 mM.

70. The method according to any one of claims 1 to 50, wherein,
step (b) further comprises, after culturing the cells in the presence of the BMP pathway activator, culturing the cells for at least 10 days in the absence of the BMP pathway activator;
step (c) comprises replating the cells of step (b) having a cobblestone morphology; and said method further comprising the step of:
(d) culturing the replated cells of step (c).

71. The method according to claim 70 wherein, in step (b), the cells are cultured for at least 20 days in the absence of BMP pathway activator.

72. The method according to claim 70 or 71 wherein, in step (b), the cells are cultured for between 30 and 50 days in the absence of BMP pathway activator.

73. The method according to any one of claims 70 to 72 wherein, in step (b), the cells are cultured for about 40 days in the absence of BMP pathway activator.

74. The method according to any one of claims 70 to 73 wherein, in step (c), the cells are replated at a density of at least 1000 cells/cm².

75. The method according to any one of claims 70 to 74 wherein, in step (c), the cells are replated at a density of between 50000 and 500000 cells/cm².

76. The method according to any one of claims 70 to 75 wherein, in step (c), the cells are replated at a density of about 200000 cells/cm².

77. The method according to any one of claims 70 to 76 wherein, in step (c), said cells are replated on Matrigel®, fibronectin or Cellstart®.

78. The method according to anyone of claims 70 to 77 wherein, in step (d), the cells are cultured for at least 5 days.

79. The method according to anyone of claims 70 to 78 wherein, in step (d), the cells are cultured for between 10 and 40 days.

80. The method according to anyone of claims 70 to 79 wherein, in step (d), the cells are cultured for about 14 days.

81. The method according to any one of claims 70 to 80 wherein, in step (d), the cells are cultured in the presence of cAMP.

82. The method according to claim 81 wherein, in step (d), the concentration of cAMP is about 0.5 mM.

83. The method according to any one of claims 70 to 82 comprising the following additional steps:
(e) replating the cells of step (d);
(f) culturing the replated cells of step (e).

84. The method according to claim 83 wherein, in step (e), the cells are replated at a density of at least 1000 cells/cm².

85. The method according to claim 83 or 84 wherein, in step (e), the cells are replated at a density of between 50000 and 500000 cells/cm².

86. The method according to any one of claims 83 to 85 wherein, in step (e), the cells are replated at a density of about 200000 cells/cm².

87. The method according to any one of claims 70 to 86, wherein, in step (e), said cells are replated on Matrigel®, fibronectin or Cellstart®.

88. The method according to anyone of claims 70 to 87 wherein, in step (f), the cells are cultured for at least 10 days.

89. The method according to anyone of claims 70 to 88 wherein, in step (f), the cells are cultured for between 15 and 40 days.

90. The method according to anyone of claims 70 to 89 wherein, in step (f), the cells are cultured for about 28 days.

91. The method according to any one of claims 1 to 90 wherein said method further comprises the step of harvesting the RPE cells.

92. The method according to any one of claims 1 to 91 wherein said method further comprises the step of purifying the RPE cells.

93. The method according to any one of claims 1 to 91 wherein said method further comprises the step of purifying the RPE cells by Fluorescence Activated Cell Sorting (FACS) or Magnetic Activated Cell Sorting (MACS).

94. The method according to claim 92 wherein said step of purifying the RPE cells comprises the step of:
contacting the cells with an anti-CD59 antibody conjugated to a fluorophore, and,
selecting the cells that bind to the anti-CD59 antibody using FACS.

95. The method according to claim 92 wherein said step of purifying the RPE cells comprises the step of:
contacting the cells with an anti-CD59 antibody conjugated to a magnetic particle, and,
selecting the cells that bind to the anti-CD59 antibody using MACS.

96. The method according to any one of claims 1 to 90 wherein, in all steps, the cells are cultured as a monolayer.

97. The method according to any one of claims 1 to 96 wherein the RPE cells are expanded by a method comprising
replating RPE cells; and,
culturing the replated RPE cells.

98. The method according to claim 97 wherein the cells are replated at a density between 1000 and 100000 cells/cm².

99. The method according to claim 97 or 98 wherein the cells are replated at a density between 10000 and 30000 cells/cm².

100. The method according to any one of claims 97 to 99 wherein the cells are replated at a density of about 20000 cells/cm².

101. The method according to any one of claims 97 to 100 wherein the cells are replated on Matrigel®, Fibronectin or Cellstart®.

102. The method according to any one of claims 97 to 101, wherein the cells are cultured for at least 7 days, at least 14 days, at least 28 days or at least 42 days.

103. The method according to any one of claims 97 to 102, wherein the cells are cultured for about 49 days.

104. The method according to any one of claims 97 to 103, wherein the cells are cultured in the presence of a SMAD inhibitor, cAMP or an agent which increases the intracellular concentration of cAMP.

105. The method according to claim 104, wherein said agent is selected from an Adenyl Cyclase activator, preferably forskolin or a phosphodiesterase (PDE) inhibitor, preferably a PDE1, PDE2, PDE3, PDE4, PDE7, PDE8, PDE10 and/or PDE11 inhibitor.

106. The method according to claim 104 or 105, wherein said the cells are cultured in the presence of cAMP.

107. The method according to claim 106, wherein the concentration of cAMP is between 0.01 mM and 1M.

108. The method according to claim 106 or 107, wherein the concentration of cAMP is about 0.5 mM.

109. A method for expanding RPE cells comprising the following steps:
(a) plating RPE cells at a density of at least 1000 cells/cm², and,
(b) culturing said RPE cells in the presence of SMAD inhibitor, cAMP or an agent which increases the intracellular concentration of cAMP.

110. The method according to claim 109, wherein, in step (a), the cells are plated at a density between 5000 and 100000 cells/cm².

111. The method according to claim 109 or 110, wherein, in step (a), the cells are plated at a density about 20000 cells/cm².

112. The method according to any one of claims 109 to 111, wherein, in step (a), the cells are plated on Matrigel®, Fibronectin or Cellstart®.

113. The method according to any one of claims 109 to 112, wherein, in step (b), the cells are cultured for at least 7 days, at least 14 days, at least 28 days or at least 42 days.

114. The method according to any one of claims 109 to 113, wherein, in step (b), the cells are cultured for about 49 days.

115. The method according to any one of claims 109 to 114, wherein said agent is selected from an adenyl Cyclase activator, preferably forskolin or a phosphodiesterase (PDE) inhibitor, preferably a PDE1, PDE2, PDE3, PDE4, PDE7, PDE8, PDE10 and/or PDE11 inhibitor.

116. The method according to any one of claims 109 to 114, wherein, in step (b), the cells are cultured in the presence of cAMP.

117. The method according to claim 116, wherein the concentration of cAMP is between 0.01 mM and 1M.

118. The method according to claim 116 or 117, wherein the concentration of cAMP is about 0.5 mM.

119. The method according to any one of claims 109 to 114, wherein, in step (b), the cells are cultured in the presence of a SMAD inhibitor.

120. The method according to claim 119, wherein the SMAD inhibitor is 2-(6-methylpyridin-2-yl)-N-(pyridin-4-yl)quinazolin-4-amine, 6-(1-(6-methylpyridin-2-yl)-1H-pyrazol-5-yl)quinazolin-4(3H)-one, or 4-methoxy-6-(3-(6-methylpyridin-2-yl)-1H-pyrazol-4-yl)quinoline.

121. The method according to any one of claims 1 to 120 wherein the produced RPE cells have a cobblestone morphology, are pigmented and express at least one of the following RPE markers: MITF, PMEL17, CRALBP, MERTK, BEST1 and ZO-1.

122. The method according to any one of claims 1 to 121 wherein the produced RPE cells secrete VEGF and PEDF.

123. The method according to any one of claims 1 to 122 wherein all steps are carried out in xeno-free conditions.

124. RPE cells obtained by a method according to anyone of claims 1 to 123.

125. RPE cells obtainable by a method according to anyone of claims 1 to 123.

126. A pharmaceutical composition comprising the RPE cells of claim 124 or 125.

127. A method for the treatment of a retinal disease in a subject, said method comprising administering RPE cells of claim 124 or 125 or a pharmaceutical composition of claim 126 to said subject.

128. A method for producing RPE cells comprising:
a) providing a population of pluripotent cells;
b) inducing the differentiation of pluripotent cells into RPE cells, and,
c) enriching the cell population for cells expressing CD59.

129. The method according to claim 128 wherein step c) comprises
contacting the cells with an anti-CD59 antibody conjugated to a fluorophore, and,
selecting the cells that bind to the anti-CD59 antibody using FACS.

130. The method according to claim 128 wherein step c) comprises
contacting the cells with an anti-CD59 antibody conjugated to a magnetic particle, and,
selecting the cells that bind to the anti-CD59 antibody using MACS.

131. A method for purifying RPE cells comprising:
a) providing a cell population comprising RPE cells and non RPE cells;
b) increasing the percentage of RPE cells in the cell population by enriching the cell population for cells expressing CD59.

132. The method according to claim 131 wherein step b) comprises
contacting the cell population with an anti-CD59 antibody conjugated to a fluorophore, and,
selecting the cells that bind to the anti-CD59 antibody using FACS.

133. The method according to claim 131 wherein step b) comprises
contacting the cell population with an anti-CD59 antibody conjugated to a magnetic particle, and,
selecting the cells that bind to the anti-CD59 antibody using MACS.

134. The method according to anyone of claims 131 to 133 wherein the non RPE cells are pluripotent cells or RPE progenitors.
